(19) United States
(12) Patent Application Publication
(10) Pub. No.: US 2026/0015621 A1
(43) Pub. Date: Jan. 15, 2026

(54) COMPOSITIONS AND METHODS FOR INCREASED PROTEIN PRODUCTION IN BACILLUS LICHENIFORMIS

(71) Applicant: DANISCO US INC., Palo Alto, CA (US)

(72) Inventors: Marc Anton Bernhard Kolkman, Oegstgeest (NL); Steven D. Doig, Wilmington, DE (US); Ryan L. Frisch, Newark, DE (US); Hongxian He, Wilmington, DE (US); Frank Wouter Koopman, Utrecht (NL); Chris Leeflang, Twisk (NL)

(21) Appl. No.: 19/337,502

(22) Filed: Sep. 23, 2025

Related U.S. Application Data
(62) Division of application No. 18/524,856, filed on Nov. 30, 2023, now Pat. No. 12,448,626.
Application No. 18/524,856 is a division of application No. 16/486,892, filed on Aug. 19, 2019, now Pat. No. 11,866,713.
(60) Provisional application No. 62/463,268, filed on Feb. 24, 2017.

(51) Int. Cl. C12N 15/75; C07K 14/32; C12P 21/02

(52) U.S. Cl. CPC C12N 15/75; C07K 14/32; C12P 21/02

(57) ABSTRACT

The present disclosure is generally related to compositions and methods for obtaining Bacillus licheniformis cells/strains having increased protein production capabilities. Certain embodiments of the disclosure are related to genetically modified Bacillus licheniformis cells/strains derived from parental B. licheniformis cells/strains comprising a variant rghR2 gene.

### CROSS-REFERENCE TO RELATED APPLICATIONS

The present application is a divisional of U.S. application Ser. No. 18/524,856, filed Nov. 30, 2023, which is a divisional of U.S. application Ser. No. 16/486,892, filed Aug. 19, 2019, now U.S. Pat. No. 11,866,713, which is a U.S. National Stage Application under 35 U.S.C. § 371 of International Application No. PCT/US2018/019140, filed Feb. 22, 2018, which claims priority to U.S. Provisional Patent Application Ser. No. 62/463,268, filed Feb. 24, 2017, all of which are hereby incorporated by reference in their entirety.

### FIELD

The present disclosure is generally related to the fields of bacteriology, microbiology, genetics, molecular biology, enzymology, industrial protein production the like. More particularly, the present disclosure is related to compositions and methods for obtaining Bacillus licheniformis cells/strains (e.g., a protein production host; cell factory) having increased protein production capabilities. Thus, certain embodiments of the disclosure are related to modified B. licheniformis cells derived from parental B. licheniformis cells comprising a chromosomal rghR2 gene (variant) encoding a RghR2 protein of SEQ ID NO: 4, wherein the modified cells comprise a genetic modification of the rghR2 gene which encodes a RghR2 protein of SEQ ID NO: 2.

### REFERENCE TO A SEQUENCE LISTING

The contents of the electronic submission of the text file Sequence Listing, named “NB41203USPCD_SequenceListing” was created on Oct. 31, 2023 and is 205,886 bytes in size, which is hereby incorporated by reference in its entirety.

### BACKGROUND

Gram-positive bacteria such as Bacillus subtilis, Bacillus licheniformis and Bacillus amyloliquefaciens are frequently used as microbial factories for the production of industrial relevant proteins, due to their excellent fermentation properties and high yields (e.g., up to 25 grams per liter culture; Van Dijl and Hecker, 2013). For example, B. subtilis is well known for its production of α-amylases (Jensen et al., 2000; Raul et al., 2014) and proteases (Brode et al., 1996) necessary for food, textile, laundry, medical instrument cleaning, pharmaceutical industries and the like (Westers et al., 2004). Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority, and many of their products gained a “Generally Recognized As Safe” (GRAS) status from the US Food and Drug Administration (Olempska-Beer et al., 2006; Earl et al., 2008; Caspers et al., 2010).

Thus, the production of proteins (e.g., enzymes, antibodies, receptors, etc.) in microbial host cells is of particular interest in the biotechnological arts. Likewise, the optimization of Bacillus host cells for the production and secretion of one or more protein(s) of interest is of high relevance, particularly in the industrial biotechnology setting, wherein small improvements in protein yield are quite significant when the protein is produced in large industrial quantities. More particularly, B. licheniformis is a Bacillus species host cell of high industrial importance, and as such, the ability to modify and engineer B. licheniformis host cells for enhanced/increased protein expression/production is highly desirable for construction of new and improved B. licheniformis production strains. The present disclosure is thus related to the highly desirable and unmet need for obtaining and constructing B. licheniformis cells (e.g., protein production host cells) having increased protein production capabilities.

### SUMMARY

The present disclosure is generally related to compositions and methods for obtaining B. licheniformis cells (e.g., a protein production host; cell factory) having increased protein production capabilities. Certain embodiments of the disclosure are related to a modified Bacillus licheniformis cell derived from a parental B. licheniformis cell comprising a rghR2 gene encoding a RghR2 protein comprising 90% sequence identity to SEQ ID NO: 4, wherein the modified cell comprises a genetic modification of the rghR2 gene which encodes a RghR2 protein comprising 90% sequence identity to SEQ ID NO: 2, wherein the modified host cell produces an increased amount of a protein of interest (relative to the unmodified parental cell). In certain embodiments, the RghR2 protein comprising 90% sequence identity to SEQ ID NO: 4 is substantially inactive as a transcriptional regulatory protein, relative to the RghR2 protein comprising 90% sequence identity to SEQ ID NO: 2. In other embodiments, the parental cell comprising the rghR2 gene encoding the RghR2 protein comprising 90% sequence identity to SEQ ID NO: 4 comprises an 18-nucleotide duplication in the rghR2 gene (rghR2_{dup}) which corresponds to a repeat of amino acids AAAISR at amino acid positions 38-43 of the RghR2 protein comprising 90% sequence identity to SEQ ID NO: 4, and wherein the modified cell comprises a modification which deletes of the 18-nucleotide duplication in the rghR2 gene (rghR2ᵣₑₛₜ), thereby encoding a RghR2 protein comprising 90% sequence identity to SEQ ID NO: 2. In certain other embodiments, the rghR2 gene encoding the RghR2 protein comprising 90% sequence identity to SEQ ID NO: 4 comprises a nucleic acid sequence having at least 90% sequence identity to SEQ ID NO:3 and comprises an 18 nucleotide duplication encoding a repeat of amino acids AAAISR at amino acid positions 38-43 of the RghR2 protein of SEQ ID NO: 4. In other embodiments, the rghR2 gene encoding the RghR2 protein of SEQ ID NO: 2 comprises a nucleic acid sequence comprising 90% sequence identity to the rghR2 gene of SEQ ID NO: 1. In yet another embodiment, the increased amount of a protein of interest is at least 1.0% increased relative to the parental cell. In certain other embodiments, the modified cell further comprising a genetic modification which disrupts, deletes, inactivates or down-regulates at least one endogenous B. licheniformis gene selected from the group consisting of abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, rghR1, spo0F, ywjG, ywq12, BLi04199, BLi04200, licT, bg1H and bg1P. In certain embodiments, the modified cell comprises a genetic modification which deletes, disrupts or down-regulates an endogenous B. licheniformis gene selected from yvzC, Bli03644, AbrB1 and abh (AbrB2), or at least two endogenous B. licheniformis genes selected from the group consisting of yvzC, Bli03644, AbrB1 and abh (AbrB2), or at least three endogenous B. licheniformis genes selected from the group consisting of yvzC, Bli03644, AbrB1 and abh (AbrB2) or all four endogenous B. licheniformis genes yvzC, Bli03644, AbrB1 and abh (AbrB2). In another embodiment, the increased amount of a protein of interest is a heterologous protein. Thus, in certain embodiments, the modified cell comprises an expression construct encoding a heterologous protein of interest. Such expression constructs encoding heterologous proteins of interest may be introduced (e.g., transformed) into the parental B. licheniformis cell prior to the one or more genetic modifications described above, introduced (e.g., transformed) into the modified B. licheniformis (daughter) cell during the one or more genetic modifications described above, or introduced (e.g., transformed) into the modified B. licheniformis (daughter) after performing the one or more genetic modifications described above. In certain other embodiments, the protein of interest is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases.

In another embodiment, the disclosure is directed to a modified B. licheniformis cell derived from a parental B. licheniformis cell comprising a rghR2_{dup} gene encoding a RghR2 protein of SEQ ID NO: 4, wherein the modified cell comprises a rghR2ᵣₑₛₜ gene encoding a RghR2 protein of SEQ ID NO: 2, wherein the modified cell produces an increased amount of a protein of interest relative to the unmodified parental cell.

In certain other embodiments, the disclosure is related to a modified B. licheniformis cell derived from a parental B. licheniformis cell comprising a rghR2 gene encoding a RghR2 protein comprising 90% sequence identity to SEQ ID NO: 4, wherein the modified cell comprises a polynucleotide construct introduced therein comprising a 5′ promoter region operably linked to a nucleic acid sequence encoding a RghR2 protein comprising 90% sequence identity to SEQ ID NO: 2, wherein the modified cell produces an increased amount of a protein of interest relative to the unmodified parental cell. In certain embodiments, the RghR2 protein comprising 90% sequence identity to SEQ ID NO: 4 is substantially inactive as a transcriptional regulatory protein relative to the RghR2 protein comprising 90% sequence identity to SEQ ID NO: 2. In another embodiment, the parental cell comprising the rghR2 gene encoding the RghR2 protein comprising 90% sequence identity to SEQ ID NO: 4 comprises an 18-nucleotide duplication in the rghR2 gene which corresponds to a repeat of amino acids AAAISR at amino acid positions 38-43 of the RghR2 protein comprising 90% sequence identity to SEQ ID NO: 4. In other embodiments, the modified cell further comprises a genetic modification which deletes, disrupts, inactivates or down-regulates the endogenous rghR2 gene encoding the RghR2 protein comprising 90% sequence identity to SEQ ID NO: 4. In other embodiments, the rghR2 gene encoding the RghR2 protein comprising 90% sequence identity to SEQ ID NO: 2 comprises a nucleic acid sequence having at least 90% sequence identity to SEQ ID NO: 1. In another embodiment, the modified cell further comprises a genetic modification which deletes, disrupts or down-regulates at least one endogenous B. licheniformis gene selected from the group consisting of yvzC, Bli03644, AbrB1 and abh (AbrB2). In certain other embodiments, the polynucleotide construct comprising a 5′ promoter region operably linked to a nucleic acid sequence encoding a RghR2 protein comprising 90% sequence identity to SEQ ID NO: is comprised within a vector. In certain embodiments, the vector is a plasmid. In certain other embodiments, the vector is integrated into the B. licheniformis genome. In another embodiment, the vector integrates into the B. licheniformis genome at the native rghR2 chromosomal locus, thereby deleting or disrupting the gene encoding the RghR2 protein comprising 90% sequence identity to SEQ ID NO: 4 and inserting therefore the introduced polynucleotide construct comprising the 5′ promoter region operably linked to the nucleic acid sequence encoding the RghR2 protein comprising 90% sequence identity to SEQ ID NO: 2. In other embodiments, the increased amount of a protein of interest is a heterologous protein. In another embodiment, the modified cell comprises an expression construct encoding a heterologous protein of interest. In certain embodiments, a protein of interest is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases and hexose oxidases. In other embodiments, the increased amount of a protein of interest is at least 1.0% increased relative to the parental host cell. In another embodiment, the modified cell further comprises an expression construct comprising allele glcT1 (SEQ ID NO: 144), encoding a variant GlcT protein comprising a Leucine (L) to Phenylalanine (F) substitution at amino acid position 67 of the variant GlcT protein.

In other embodiments, the disclosure is related to a modified B. licheniformis cell derived from a parental B. licheniformis cell, the modified cell comprising a genetic modification which deletes, disrupts or down-regulates an endogenous B. licheniformis yvzC gene encoding a YvzC protein comprising 90% sequence identity to SEQ ID NO: 18, wherein the modified cell produces an increased amount of a protein of interest relative to the unmodified parental cell.

In other embodiments, the disclosure is related to a modified B. licheniformis cell derived from a parental B. licheniformis cell, the modified cell comprising a genetic modification which deletes, disrupts or down-regulates an endogenous B. licheniformis Bli03644 gene encoding a Bli03644 protein comprising 90% sequence identity to SEQ ID NO: 20, wherein the modified cell produces an increased amount of a protein of interest relative to the unmodified parental cell.

In other embodiments, the disclosure is related to a modified B. licheniformis cell derived from a parental B. licheniformis cell, the modified cell comprising a genetic modification which deletes, disrupts or down-regulates an endogenous B. licheniformis AbrB1 gene encoding a AbrB1 protein comprising 90% sequence identity to SEQ ID NO: 22, wherein the modified cell produces an increased amount of a protein of interest relative to the unmodified parental cell.

In other embodiments, the disclosure is related to a modified B. licheniformis cell derived from a parental B. licheniformis cell, the modified cell comprising a genetic modification which deletes, disrupts or down-regulates an endogenous B. licheniformis abh (AbrB2) gene encoding a abh (AbrB2) protein comprising 90% sequence identity to SEQ ID NO: 24, wherein the modified cell produces an increased amount of a protein of interest relative to the unmodified parental cell.

In certain other embodiments, the disclosure is directed to a modified B. licheniformis cell derived from a parental B. licheniformis cell comprising a rghR2 gene encoding a RghR2 protein comprising 90% sequence identity to SEQ ID NO: 2, wherein the modified cell comprises a genetic modification which deletes, disrupts or down-regulates at least one endogenous B. licheniformis gene selected from the group consisting of yvzC, Bli03644, AbrB1 and abh (AbrB2), wherein the modified cell produces an increased amount of a protein of interest relative to the unmodified parental cell. In certain embodiments, the rghR2 gene encoding the RghR2 protein comprising 90% sequence identity to SEQ ID NO: 2, comprises a nucleic acid sequence having at least 90% sequence identity to SEQ ID NO: 1. In another embodiment, the aforementioned modified cells further comprise an expression construct comprising allele glcT1 (SEQ ID NO: 144), encoding a variant GlcT protein comprising a Leucine (L) to Phenylalanine (F) substitution at amino acid position 67 of the variant GlcT protein.

In another embodiment, the disclosure is related to a modified B. licheniformis cell derived from a parental B. licheniformis cell comprising a rghR2 gene encoding a RghR2 protein comprising 90% sequence identity to SEQ ID NO: 4, wherein the modified cell comprises a genetic modification which deletes, disrupts or down-regulates at least one endogenous B. licheniformis gene selected from the group consisting of yvzC, Bli03644, AbrB1 and abh (AbrB2), wherein the modified cell produces an increased amount of a protein of interest relative to the unmodified parental cell. In certain embodiments, the rghR2 gene encoding the RghR2 protein comprising 90% sequence identity to SEQ ID NO: 4, comprises a nucleic acid sequence having at least 90% sequence identity to SEQ ID NO: 3. In other embodiments, the RghR2 protein comprising 90% sequence identity to SEQ ID NO: 4 is substantially inactive as a transcriptional regulatory protein relative to the RghR2 protein comprising 90% sequence identity to SEQ ID NO: 2. In other embodiments, the parental cell comprising the rghR2 gene encoding the RghR2 protein comprising 90% sequence identity to SEQ ID NO: 4, comprises an 18-nucleotide duplication in the rghR2 gene which corresponds to a repeat of amino acids AAAISR at amino acid positions 38-43 of the RghR2 protein comprising 90% sequence identity to SEQ ID NO: 4. In certain other embodiments the increased expression of a protein of interest is a heterologous protein of interest.

In certain other embodiments, the disclosure is related to a method for restoring the activity of a substantially inactive RghR2 protein in a parental B. licheniformis cell, wherein the parental cell comprises a rghR2 gene encoding a substantially inactive RghR2 protein comprising 90% sequence identity to SEQ ID NO: 4, the method comprising: (a) obtaining a parental B. licheniformis cell comprising a gene encoding a RghR2 protein comprising 90% sequence identity to SEQ ID NO: 4, wherein gene encoding the RghR2 protein comprising 90% sequence identity to SEQ ID NO: 4 comprises an 18-nucleotide duplication in the rghR2 gene which corresponds to a repeat of amino acids AAAISR at amino acid positions 38-43 of the RghR2 protein of SEQ ID NO: 4, and (b) modifying the cell of step (a) by deleting the 18-nucleotide duplication in the rghR2 gene to yield a rghR2ᵣₑₛₜ gene, wherein the rghR2ᵣₑₛₜ gene thereby encodes an active RghR2 protein comprising 90% sequence identity to SEQ ID NO: 2. In certain embodiments of the method, the modified cell of step (b) further comprises an introduced polynucleotide expression construct encoding a heterologous protein of interest. In another embodiment of the method, deleting the 18-nucleotide duplication in the rghR2 gene of step (b) comprises deleting the nucleotide duplication by a method selected from homologous recombination, site directed mutagenesis, CRISPR-Cas9 gene editing, TALEN gene editing, homing endonuclease gene editing and ZFN gene editing. In certain embodiments, the rghR2 gene encoding the RghR2 protein comprising 90% sequence identity to SEQ ID NO: 4 comprises a nucleic acid sequence comprising 90% sequence identity to SEQ ID NO: 3, wherein the 18-nucleotide duplication at nucleotides 111-129 of SEQ ID NO: 3 are deleted. In other embodiments, the modified cell of step (b) further comprises a genetic modification which deletes, disrupts, or down-regulates at least one endogenous B. licheniformis gene selected from the group consisting of yvzC, Bli03644, AbrB1 and abh (AbrB2). In certain embodiments of the method, the genetic modification which deletes at least one endogenous B. licheniformis gene is a complete gene deletion or a partial deletion. In certain embodiments, a partial gene deletion comprises deleting the gene's operator, deleting the gene's promoter, deleting the gene's enhancer, deleting the gene's 5′ UTR, deleting the gene's start codon, deleting the gene's encoded ribosomal binding site (RBS), deleting the gene's 3′ UTR, deleting the 10% of the gene's coding sequence, deleting the 25% of the gene's coding sequence, deleting the 50% of the gene's coding sequence, deleting the 75% of the gene's coding sequence or any combination thereof.

In another embodiment, the disclosure is related to a method for restoring the activity of a substantially inactive RghR2 protein in a parental B. licheniformis cell, wherein the parental cell comprises a rghR2_{dup} gene encoding a substantially inactive RghR2 protein comprising 90% sequence identity to SEQ ID NO: 4, the method comprising: (a) obtaining a parental B. licheniformis cell comprising a rghR2_{dup} gene encoding a RghR2 protein comprising at least 90% sequence identity to SEQ ID NO: 4, wherein the rghR2_{dup} gene encoding the RghR2 protein of SEQ ID NO: 4 comprises an 18-nucleotide duplication in the rghR2 gene which corresponds to a repeat of amino acids AAAISR at amino acid positions 38-43 of the RghR2 protein of SEQ ID NO: 4, (b) modifying the parental cell of step (a) by introducing therein a polynucleotide construct comprising a 5′ promoter region operably linked to a rghR2ᵣₑₛₜ nucleic acid sequence encoding a RghR2 protein comprising 90% sequence identity to SEQ ID NO: 2, and (c) expressing the polynucleotide construct introduced into the modified cell of step (b) encoding the active RghR2 protein comprising 90% sequence identity to SEQ ID NO: 2. In particular embodiments, the modified cell comprises an introduced polynucleotide expression construct encoding a heterologous protein of interest. In another embodiment, the modified cell comprises a genetic modification which deletes, disrupts, or down-regulates at least one endogenous B. licheniformis gene selected from the group consisting of yvzC, Bli03644, AbrB1 and abh (AbrB2). In another embodiment, the genetic modification which deletes at least one endogenous B. licheniformis gene is a complete gene deletion or a partial deletion. In certain embodiments, a partial gene deletion comprises deleting the gene's operator, deleting the gene's promoter, deleting the gene's enhancer, deleting the gene's 5′ UTR, deleting the gene's start codon, deleting the gene's encoded ribosomal binding site (RBS), deleting the gene's 3′ UTR, deleting the 10% of the gene's coding sequence, deleting the 25% of the gene's coding sequence, deleting the 50% of the gene's coding sequence, deleting the 75% of the gene's coding sequence or any combination thereof.

In other embodiments, the disclosure is related to a method for increasing the production of an endogenous protein of interest in B. licheniformis cells comprising: (a) obtaining a parental B. licheniformis cell comprising a gene encoding a RghR2 protein comprising at least 90% sequence identity to SEQ ID NO: 4, wherein gene encoding the RghR2 protein of SEQ ID NO: 4 comprises an 18-nucleotide duplication in the rghR2 gene which corresponds to a repeat of amino acids AAAISR at amino acid positions 38-43 of the RghR2 protein of SEQ ID NO: 4, (b) modifying the cell of step (a) by deleting the 18-nucleotide duplication in the rghR2 gene, wherein the rghR2 gene thereby encodes a RghR2 protein comprising 90% sequence identity to SEQ ID NO: 2, (c) cultivating the modified cell of step (b) in a medium suitable for the production of the endogenous protein, and (d) recovering the endogenous protein produced in step (c) from the cultivation medium or cell lysate, wherein the modified B. licheniformis cell of step (b) produces an increased amount of the endogenous protein, relative to the parental B. licheniformis cell obtained in step (a), when both the cells of step (a) and the cells of step (b) are cultivated under the same conditions. In certain embodiments, the rghR2 gene encoding the RghR2 protein comprising 90% sequence identity to SEQ ID NO: 4 comprises a nucleic acid sequence comprising 90% sequence identity to SEQ ID NO: 3, wherein the 18-nucleotide duplication at nucleotides 111-129 of SEQ ID NO: 3 are deleted. In certain embodiments, the genetic modification which deletes at least one endogenous B. licheniformis gene is a complete gene deletion or a partial gene deletion. In certain embodiments, a partial gene deletion comprises deleting the gene's operator, deleting the gene's promoter, deleting the gene's enhancer, deleting the gene's 5′ UTR, deleting the gene's start codon, deleting the gene's encoded ribosomal binding site (RBS), deleting the gene's 3′ UTR, deleting 10% of the gene's coding sequence, deleting 25% of the gene's coding sequence, deleting 50% of the gene's coding sequence, deleting 75% of the gene's coding sequence or any combination thereof.

In yet other embodiments the disclosure is directed to a method for increasing the production of a heterologous protein of interest in B. licheniformis cells comprising: (a) obtaining a parental B. licheniformis cell comprising a gene encoding a RghR2 protein comprising at least 90% sequence identity to SEQ ID NO: 4, wherein gene encoding the RghR2 protein of SEQ ID NO: 4 comprises an 18-nucleotide duplication in the rghR2 gene which corresponds to a repeat of amino acids AAAISR at amino acid positions 38-43 of the RghR2 protein of SEQ ID NO: 4, (b) modifying the cell of step (a) by deleting the 18-nucleotide duplication in the rghR2 gene, wherein the rghR2 gene thereby encodes a RghR2 protein comprising 90% sequence identity to SEQ ID NO: 2, (c) introducing an expression construct encoding a heterologous protein into the modified B. licheniformis cell of step (b), (d) cultivating the modified cell of step (c) in a medium suitable for the production of the heterologous protein, and (e) recovering the heterologous protein produced in step (d) from the cultivation medium or cell lysate, wherein the modified B. licheniformis cell of step (b) produces an increased amount of the heterologous protein relative to the parental B. licheniformis cell obtained in step (a), comprising the same introduced expression construct encoding the heterologous protein, when both the cells of step (a) and cells of step (b) are cultivated under the same conditions. In certain embodiments, the rghR2 gene encoding the RghR2 protein comprising 90% sequence identity to SEQ ID NO: 4 comprises a nucleic acid sequence comprising 90% sequence identity to SEQ ID NO: 3, wherein the 18-nucleotide duplication at nucleotides 111-129 of SEQ ID NO: 3 are deleted.

In certain other embodiments, the disclosure is related to a method for restoring the activity of a substantially inactive RghR2 protein in a parental B. licheniformis cell, wherein the parental cell comprises a rghR2 gene encoding a substantially inactive RghR2 protein comprising 90% sequence identity to SEQ ID NO: 4, the method comprising: (a) obtaining a parental B. licheniformis cell comprising a gene encoding a RghR2 protein comprising at least 90% sequence identity to SEQ ID NO: 4, wherein gene encoding the RghR2 protein of SEQ ID NO: 4 comprises an 18-nucleotide duplication in the rghR2 gene which corresponds to a repeat of amino acids AAAISR at amino acid positions 38-43 of the RghR2 protein of SEQ ID NO: 4, and (b) modifying the parental cell of step (a) by: (i) deleting the 18-nucleotide duplication in the rghR2 gene, or (ii) by deleting, disrupting, or down-regulating the rghR2 gene, and (c) deleting, disrupting, or down-regulating at least one endogenous B. licheniformis gene selected from the group consisting of yvzC, Bli03644, AbrB1 and abh (AbrB2), wherein the modified cell produces an increased amount of a protein of interest relative to the unmodified parental cell.

In other embodiments, the disclosure is directed to isolated (modified) B. licheniformis cell produced by the methods disclosed herein.

In other embodiments, the disclosure is related to a method for identifying a B. licheniformis strain comprising a variant rghR2 gene encoding a substantially inactive RghR2 protein, the method comprising: (a) obtaining a B. licheniformis strain and sequencing the rghR2 gene therein, (b) aligning and comparing the sequenced rghR2 gene with the native rghR2 gene of SEQ ID NO: 1, wherein a B. licheniformis strain comprising a sequenced rghR2 gene comprising an insertion, deletion, substitution and/or duplication of one or more nucleotides in the HTH domain of the rghR2 gene of SEQ ID NO: 1 comprises a variant rghR2 gene encoding a substantially inactive RghR2 protein. In certain embodiments, the HTH domain of a native rghR2 protein of SEQ ID NO: 2 is comprised within amino acid residues 5-58 of SEQ ID NO: 2. In another embodiment, the insertion of one or more nucleotides in the HTH domain of the rghR2 gene of is between nucleotides 111 and 112 of SEQ ID NO: 1. In another embodiment, the B. licheniformis strain comprising a variant rghR2 gene encoding a substantially inactive RghR2 protein comprises a six amino acid repeat present in the RghR2 variant protein of SEQ ID NO: 4.

Thus, certain embodiments of the disclosure are related to modified Bacillus licheniformis cells (i.e., daughter cells) derived from parental B. licheniformis cells, wherein the modified (daughter) cells are capable of expressing/producing increased amounts of one or more proteins of interest, particularly industrially relevant proteins (enzymes) such as amylases, proteases, lipases, esterases and the like.

### BRIEF DESCRIPTION OF DRAWINGS

FIG. 1 shows an amino acid sequence alignment of the RghR2 protein of B. licheniformis DSM13 strain and the RghR2 protein of B. licheniformis Bra7 strain. As presented in FIG. 1, the RghR2 protein from B. licheniformis isolates Bra7, a Bra7 derived isolate, T5, ATCC-6598 and ATCC-9789 (e.g., see the full length variant RghR2 protein of SEQ ID NO: 4), each comprise a direct repeat of amino acids “Ala-Ala-Ala-Ile-Ser-Arg”. For example, as presented in FIG. 1, the variant RghR2 proteins from B. licheniformis isolates Bra7, Bra7 derivative, T5, ATCC-6598 and ATCC-9789 (SEQ ID NO: 4) comprise the six (6) amino acid repeat “AAISR” inserted as follows: Ala₃₂-Ala₃₃-Ala₃₄-Ile₃₅-Ser₃₆-Arg₃₇-Ala₃₈-Ala₃₉-Ala₄₀-Ile₄₁-Ser₄₂-Arg₄₃(SEQ ID NO: 6). In contrast, as presented in FIG. 1, the native RghR2 proteins from B. licheniformis isolates such as DSM13, ATCC-27811 and DSM603, do not comprise the repeated “Ala-Ala-Ala-Ile-Ser-Arg” sequence set forth in SEQ ID NO: 6 (e.g., see full length native RghR2 protein of SEQ ID NO: 2).

FIG. 2 shows plasmid “pCZ105”, which comprises various restriction enzyme sites, amongst which are the HindIII and NotI, a pE194 temperature sensitive replicon (Ts replicon), a kanamycin coding sequence (Kan), a kanamycin promoter (pKan marker), a ribosomal terminator sequence (Term rrnB), a β-lactamase (“Bla”) gene and an I-Sce site are present.

FIG. 3 shows the engineered “pCZ105 rghR2” plasmid. This plasmid comprises a pE194 temperature sensitive replicon (Ts replicon), a kanamycin coding sequence (Kan), a kanamycin promoter (pKan marker), a ribosomal terminator sequence (Term rrnB), an I-Sce site, an eighteen base-pair (18-bp) deleted rghR2 gene and rghR2 flanking regions.

FIG. 4 shows a map of plasmid “pBLComK”. This plasmid includes DNA sequences encoding the pBR322 origin of replication, the Enterococcus faecalis Spectinomycin resistance (Spec^{r}) gene spc (also called aad9), the B. subtilis (natto) plasmid pTA1060 rep gene for replication in Bacilli, the B. licheniformis comK gene (controlled by the B. subtilis xylA promoter), and the B. subtilis xylR gene.

FIG. 5 shows a bar graph representing results of a slow release microtiter plate experiment (see, Example 2) of control (parental) B. licheniformis host cells (i.e., B. licheniformis cells comprising the 18-bp rghR2 duplication; rghR2_{dup}) and B. licheniformis clone 197 (i.e., B. licheniformis daughter cells comprising a restored rghR2 gene (rghR2ᵣₑₛₜ). More particularly, as presented in FIG. 5, the light grey bars represent the relative optical density (cell density) to the control, and the black bars represent the relative production titers of a heterologous Peanibacillus curdlanolyticus variant α-amylase relative to the control.

FIG. 6 shows the specific relative protein production (i.e., P. curdlanolyticus α-amylase) of the B. licheniformis rghR2ᵣₑₛₜ strain (i.e., daughter cell, clone 197) compared to the control (parental; rghR2_{dup}) strain.

FIG. 7 shows the (protein) production of a P. curdlanolyticus α-amylase (black bars) expressed in modified B. licheniformis host cells comprising a disrupted BLi03644 gene (ΔBLi03644), a disrupted abrB1 gene (ΔabrB1), a disrupted yvzC gene (ΔyvzC) or a disrupted abh gene (Δabh), relative to the (parental) control host cell. The OD₆₀₀ of these cell cultures are presented as grey bars in FIG. 7.

FIG. 8 shows codons in the DSM13 rghR2 gene, indicated in bold, encoding residues involved in DNA binding. The 18-bp sequence duplicated in the rghR2 gene of B. licheniformis strains Bra7, T5, ATCC-9789 and ATCC-6598 is boxed and is located in a region predicted to encode a sequence-specific DNA binding site.

FIG. 9 shows the production of a heterologous EC 3.1.1.3 enzyme comprising lipase/esterase activity. As presented in the FIG. 9 SDS-PAGE, production of the heterologous EC 3.1.1.3 enzyme comprising lipase/esterase activity is improved in the B. licheniformis rghR2ᵣₑₛₜ cells vis-à-vis the B. licheniformis cells comprising the rghR2 gene having the 18-bp duplication (rghR2_{dup}).

### BRIEF DESCRIPTION OF THE BIOLOGICAL SEQUENCES

SEQ ID NO: 1 is a nucleic acid sequence encoding a native Bacillus licheniformis RghR2 protein of SEQ ID NO: 2.

SEQ ID NO: 2 is the amino acid sequence of the native B. licheniformis RghR2 protein encoded by nucleic acid sequence of SEQ ID NO: 1.

SEQ ID NO: 3 is a variant B. licheniformis nucleic acid sequence encoding a variant B. licheniformis RghR2 protein of SEQ ID NO: 4. More particularly, the variant nucleic acid sequence of SEQ ID NO: 3 comprises an 18-base pair (bp) nucleotide duplication, which is not present in the nucleic acid sequence of SEQ ID NO: 1.

SEQ ID NO: 4 is the amino acid sequence of the variant RghR2 protein encoded by the nucleic acid sequence of SEQ ID NO: 3. More particularly, the variant RghR2 protein of SEQ ID NO: 4 comprises a six (6) amino acid residue repeat of “Ala-Ala-Ala-Ile-Ser-Arg” at amino acid residues 36-41 (of SEQ ID NO: 4), wherein the native RghR2 protein of SEQ ID NO: 2 does not comprise the six (6) amino acid residue repeat of “Ala-Ala-Ala-Ile-Ser-Arg” at amino acid residues 36-41.

SEQ ID NO: 5 shows amino acid residues 30-35 (Ala₃₀-Ala₃₁-Ala₃₂-Ile₃₃-Ser₃₄-Arg₃₅) of the native B. licheniformis RghR2 protein (i.e., encoded by SEQ ID NO: 1).

SEQ ID NO: 6 shows amino acid residues 30-41 (Ala₃₀-Ala₃₁-Ala₃₂-Ile₃₃-Ser₃₄-Arg₃₅-Ala₃₆-Ala₃₇-Ala₃₈-Ile₃₉-Ser₄₀-Arg₄₁) of the variant B. licheniformis RghR2 protein encoded by SEQ ID NO: 3), which comprises a repeat of SEQ ID NO: 5 at amino acid positions 36-41 in SEQ ID NO: 4. Thus, the 18-bp nucleotide duplication set forth in SEQ ID NO: 3 encodes a 6-amino acid repeat of Ala-Ala-Ala-Ile-Ser-Arg, which is represented herein as “Ala-Ala-Ala-Ile-Ser-Arg-Ala-Ala-Ala-Ile-Ser-Arg” as set forth in SEQ ID NO: 6.

SEQ ID NO: 7 is a nucleic acid sequence of primer 369.

SEQ ID NO: 8 is a nucleic acid sequence of primer 378.

SEQ ID NO: 9 is a nucleic acid sequence of primer 379.

SEQ ID NO: 10 is a nucleic acid sequence of primer 380.

SEQ ID NO: 11 is a nucleic acid sequence of primer 381.

SEQ ID NO: 12 is a nucleic acid sequence of primer 384.

SEQ ID NO: 13 is a nucleic acid sequence of primer 752.

SEQ ID NO: 14 is a nucleic acid sequence of primer 753.

SEQ ID NO: 15 is a nucleic acid sequence encoding a native Bacillus licheniformis RghR1 protein of SEQ ID NO: 16.

SEQ ID NO: 16 is the amino acid sequence of the native B. licheniformis RghR1 protein encoded by nucleic acid sequence of SEQ ID NO: 15.

SEQ ID NO: 17 is the nucleic acid sequence encoding the B. licheniformis YvzC protein of SEQ ID NO: 18.

SEQ ID NO: 18 is the amino acid sequence of the B. licheniformis YvzC protein encoded by nucleic acid sequence of SEQ ID NO: 17.

SEQ ID NO: 19 is the nucleic acid sequence encoding the B. licheniformis Bli03644 protein of SEQ ID NO: 20.

SEQ ID NO: 20 is the amino acid sequence of the B. licheniformis Bli03644 protein encoded by nucleic acid sequence of SEQ ID NO: 19.

SEQ ID NO: 21 is the nucleic acid sequence encoding the B. licheniformis AbrB1 protein of SEQ ID NO: 22.

SEQ ID NO: 22 is the amino acid sequence of the B. licheniformis AbrB1 protein encoded by nucleic acid sequence of SEQ ID NO: 21.

SEQ ID NO: 23 is the nucleic acid sequence encoding the B. licheniformis Abh (AbrB2) protein of SEQ ID NO: 24.

SEQ ID NO: 24 is the amino acid sequence of the B. licheniformis Abh (AbrB2) protein encoded by nucleic acid sequence of SEQ ID NO: 23.

SEQ ID NO: 25 is the nucleic acid sequence encoding the B. licheniformis RpmJ protein of SEQ ID NO: 26.

SEQ ID NO: 26 is the amino acid sequence of the B. licheniformis RpmJ protein encoded by nucleic acid sequence of SEQ ID NO: 25.

SEQ ID NO: 27 is the nucleic acid sequence encoding the B. licheniformis RpIM protein of SEQ ID NO: 28.

SEQ ID NO: 28 is the amino acid sequence of the B. licheniformis RpIM protein encoded by nucleic acid sequence of SEQ ID NO: 27.

SEQ ID NO: 29 is the nucleic acid sequence encoding the B. licheniformis BLi00412 protein of SEQ ID NO: 30.

SEQ ID NO: 30 is the amino acid sequence of the B. licheniformis BLi00412 protein encoded by nucleic acid sequence of SEQ ID NO: 29.

SEQ ID NO: 31 is the nucleic acid sequence encoding the B. licheniformis RapK protein of SEQ ID NO: 32.

SEQ ID NO: 32 is the amino acid sequence of the B. licheniformis RapK protein encoded by nucleic acid sequence of SEQ ID NO: 31.

SEQ ID NO: 33 is the nucleic acid sequence encoding the B. licheniformis PhrK protein of SEQ ID NO: 34.

SEQ ID NO: 34 is the amino acid sequence of the B. licheniformis PhrK protein encoded by nucleic acid sequence of SEQ ID NO: 33.

SEQ ID NO: 35 is the nucleic acid sequence encoding the B. licheniformis BLi00753 protein of SEQ ID NO: 36.

SEQ ID NO: 36 is the amino acid sequence of the B. licheniformis BLi00753 protein encoded by nucleic acid sequence of SEQ ID NO: 35.

SEQ ID NO: 37 is the nucleic acid sequence encoding the B. licheniformis YfjT protein of SEQ ID NO: 38.

SEQ ID NO: 38 is the amino acid sequence of the B. licheniformis YfjT protein encoded by nucleic acid sequence of SEQ ID NO: 37.

SEQ ID NO: 39 is the nucleic acid sequence encoding the B. licheniformis BLi00828 protein of SEQ ID NO: 40.

SEQ ID NO: 40 is the amino acid sequence of the B. licheniformis BLi00828 protein encoded by nucleic acid sequence of SEQ ID NO: 39.

SEQ ID NO: 41 is the nucleic acid sequence encoding the B. licheniformis YhdX protein of SEQ ID NO: 42.

SEQ ID NO: 42 is the amino acid sequence of the B. licheniformis YhdX protein encoded by nucleic acid sequence of SEQ ID NO: 41.

SEQ ID NO: 43 is the nucleic acid sequence encoding the B. licheniformis YhzC protein of SEQ ID NO: 44.

SEQ ID NO: 44 is the amino acid sequence of the B. licheniformis YhzC protein encoded by nucleic acid sequence of SEQ ID NO: 43.

SEQ ID NO: 45 is the nucleic acid sequence encoding the B. licheniformis Terf2 protein of SEQ ID NO: 46.

SEQ ID NO: 46 is the amino acid sequence of the B. licheniformis Terf2 protein encoded by nucleic acid sequence of SEQ ID NO: 45.

SEQ ID NO: 47 is the nucleic acid sequence encoding the B. licheniformis ZosA protein of SEQ ID NO: 48.

SEQ ID NO: 48 is the amino acid sequence of the B. licheniformis ZosA protein encoded by nucleic acid sequence of SEQ ID NO: 47.

SEQ ID NO: 49 is the nucleic acid sequence encoding the B. licheniformis AbbA protein of SEQ ID NO: 50.

SEQ ID NO: 50 is the amino acid sequence of the B. licheniformis AbbA protein encoded by nucleic acid sequence of SEQ ID NO: 49.

SEQ ID NO: 51 is the nucleic acid sequence encoding the B. licheniformis SpeG protein of SEQ ID NO: 52.

SEQ ID NO: 52 is the amino acid sequence of the B. licheniformis SpeG protein encoded by nucleic acid sequence of SEQ ID NO: 51.

SEQ ID NO: 53 is the nucleic acid sequence encoding the B. licheniformis YppF protein of SEQ ID NO: 54.

SEQ ID NO: 54 is the amino acid sequence of the B. licheniformis YppF protein encoded by nucleic acid sequence of SEQ ID NO: 53.

SEQ ID NO: 55 is the nucleic acid sequence encoding the B. licheniformis BLi02543 protein of SEQ ID NO: 56.

SEQ ID NO: 56 is the amino acid sequence of the B. licheniformis BLi02543 protein encoded by nucleic acid sequence of SEQ ID NO: 55.

SEQ ID NO: 57 is the nucleic acid sequence encoding the B. licheniformis MntR protein of SEQ ID NO: 58.

SEQ ID NO: 58 is the amino acid sequence of the B. licheniformis MntR protein encoded by nucleic acid sequence of SEQ ID NO: 57.

SEQ ID NO: 59 is the nucleic acid sequence encoding the B. licheniformis BLi02768 protein of SEQ ID NO: 60.

SEQ ID NO: 60 is the amino acid sequence of the B. licheniformis BLi02768 protein encoded by nucleic acid sequence of SEQ ID NO: 59.

SEQ ID NO: 61 is the nucleic acid sequence encoding the B. licheniformis SspA protein of SEQ ID NO: 62.

SEQ ID NO: 62 is the amino acid sequence of the B. licheniformis SspA protein encoded by nucleic acid sequence of SEQ ID NO: 61.

SEQ ID NO: 63 is the nucleic acid sequence encoding the B. licheniformis BLi03127 protein of SEQ ID NO: 64.

SEQ ID NO: 64 is the amino acid sequence of the B. licheniformis BLi03127 protein encoded by nucleic acid sequence of SEQ ID NO: 63.

SEQ ID NO: 65 is the nucleic acid sequence encoding the B. licheniformis BLi03635 protein of SEQ ID NO: 66.

SEQ ID NO: 66 is the amino acid sequence of the B. licheniformis BLi03635 protein encoded by nucleic acid sequence of SEQ ID NO: 65.

SEQ ID NO: 67 is the nucleic acid sequence encoding the B. licheniformis MrgA protein of SEQ ID NO: 68.

SEQ ID NO: 68 is the amino acid sequence of the B. licheniformis MrgA protein encoded by nucleic acid sequence of SEQ ID NO: 67.

SEQ ID NO: 69 is the nucleic acid sequence encoding the B. licheniformis Spo0F protein of SEQ ID NO: 70.

SEQ ID NO: 70 is the amino acid sequence of the B. licheniformis Spo0F protein encoded by nucleic acid sequence of SEQ ID NO: 69.

SEQ ID NO: 71 is the nucleic acid sequence encoding the B. licheniformis YwjG protein of SEQ ID NO: 72.

SEQ ID NO: 72 is the amino acid sequence of the B. licheniformis YwjG protein encoded by nucleic acid sequence of SEQ ID NO: 71.

SEQ ID NO: 73 is the nucleic acid sequence encoding the B. licheniformis YwgI2 protein of SEQ ID NO: 74.

SEQ ID NO: 74 is the amino acid sequence of the B. licheniformis YwgI2 protein encoded by nucleic acid sequence of SEQ ID NO: 73.

SEQ ID NO: 75 is the nucleic acid sequence encoding the B. licheniformis BLi04199 protein of SEQ ID NO: 76.

SEQ ID NO: 76 is the amino acid sequence of the B. licheniformis BLi04199 protein encoded by nucleic acid sequence of SEQ ID NO: 75.

SEQ ID NO: 77 is the nucleic acid sequence encoding the B. licheniformis BLi04200 protein of SEQ ID NO: 78.

SEQ ID NO: 78 is the amino acid sequence of the B. licheniformis BLi04200 protein encoded by nucleic acid sequence of SEQ ID NO: 77.

SEQ ID NO: 79 is the nucleic acid sequence encoding the B. licheniformis LicT protein of SEQ ID NO: 80.

SEQ ID NO: 80 is the amino acid sequence of the B. licheniformis LicT protein encoded by nucleic acid sequence of SEQ ID NO: 79.

SEQ ID NO: 81 is the nucleic acid sequence encoding the B. licheniformis BglH protein of SEQ ID NO: 82.

SEQ ID NO: 82 is the amino acid sequence of the B. licheniformis BglH protein encoded by nucleic acid sequence of SEQ ID NO: 81.

SEQ ID NO: 83 is the nucleic acid sequence encoding the B. licheniformis BglP protein of SEQ ID NO: 84.

SEQ ID NO: 84 is the amino acid sequence of the B. licheniformis BglP protein encoded by nucleic acid sequence of SEQ ID NO: 83.

SEQ ID NO: 85 is the nucleic acid sequence encoding the B. licheniformis ComK protein of SEQ ID NO: 86.

SEQ ID NO: 86: is the amino acid sequence of the B. licheniformis ComK protein encoded by nucleic acid sequence of SEQ ID NO: 85.

SEQ ID NO: 87 is the nucleotide sequence of the B. licheniformis Bra7 strain 18-bp duplication.

SEQ ID NO: 88 is the amino acid sequence of the S. pyogenes Cas9 protein.

SEQ ID NO: 89 is the amino acid sequence of the Acidominococcus sp. Cpf1 protein.

SEQ ID NO: 90 is the amino acid sequence of the N. gregoryi Ago protein.

SEQ ID NO: 91 is the nucleic acid sequence encoding the S. pyogenes Cas9 protein of SEQ ID NO: 88.

SEQ ID NO: 92 is a codon optimized nucleic acid sequence encoding the S. pyogenes Cas9 protein of SEQ ID NO: 88.

SEQ ID NO: 93 is the nucleic acid sequence of the B. subtilis aprE promoter.

SEQ ID NO: 94 is the nucleic acid sequence of the B. subtilis xylA promoter.

SEQ ID NO: 95 is a spac promoter nucleic acid sequence.

SEQ ID NO: 96 is a Hyper-spank promoter nucleic acid sequence.

SEQ ID NO: 97 is the nucleic acid sequence of the B. subtilis veg promoter.

SEQ ID NO: 98 is the nucleic acid sequence of the B. subtilis nprE promoter.

SEQ ID NO: 99 is the nucleic acid sequence of the T5 phage N25 promoter.

SEQ ID NO: 100 is the nucleic acid sequence of the B. subtilis groE promoter.

SEQ ID NO: 101 is the nucleic acid sequence of the B. subtilis AraA promoter.

SEQ ID NO: 102 is the nucleic acid sequence of the B. subtilis AraA2 promoter.

SEQ ID NO: 103 is the nucleic acid sequence of a lambda phage T0 terminator.

SEQ ID NO: 104 is a nucleic acid sequence of a Cas9 expression cassette.

SEQ ID NO: 105 is the nucleic acid sequence of the B. licheniformis (Bra7) 18-bp duplication.

SEQ ID NO: 106 is a nucleic acid sequence of a 17-bp VT.

SEQ ID NO: 107 is a nucleic acid sequence of an 18-bp VT.

SEQ ID NO: 108 is a nucleic acid sequence of a 19-bp VT.

SEQ ID NO: 109 is a nucleic acid sequence of a 20-bp VT.

SEQ ID NO: 110 is a nucleic acid sequence encoding a Cas9 endonuclease recognition domain.

SEQ ID NO: 111 is a nucleic acid sequence encoding a guide-RNA (gRNA) targeting the 18-bp duplication.

SEQ ID NO: 112 is a nucleic acid sequence encoding a gRNA expression cassette.

SEQ ID NO: 113 is a 500-bp nucleic acid sequence which is 5′ (upstream) of the 18-bp duplication.

SEQ ID NO: 114 is a 500-bp nucleic acid sequence which is 3′ (downstream) of the 18-bp duplication.

SEQ ID NO: 115 is a rghR2 (18-bp duplication) editing template nucleic acid sequence.

SEQ ID NO: 116 is the B. licheniformis (Bra7) nucleic acid sequence comprising the rghR2 gene.

SEQ ID NO: 117 is a forward primer sequence directed to the rghR2 gene locus.

SEQ ID NO: 118 is a reverse primer sequence directed to the rghR2 gene locus.

SEQ ID NO: 119 is a nucleic acid sequence comprising the edited rghR2 locus.

SEQ ID NO: 120 is an rghR2 sequencing primer.

SEQ ID NO: 121 is a B. licheniformis yvc target site 1 nucleic acid sequence.

SEQ ID N0122 is a B. licheniformis yvc target site 2 nucleic acid sequence.

SEQ ID NO: 123 is a B. licheniformis yvc target site 3 nucleic acid sequence.

SEQ ID NO: 124 is a B. licheniformis yvc target site 4 nucleic acid sequence.

SEQ ID NO: 125 is a B. licheniformis yvc target site 5 nucleic acid sequence.

SEQ ID NO: 126 is a B. licheniformis yvc target site 6 nucleic acid sequence.

SEQ ID NO: 127 is a B. licheniformis yvc target site 7 nucleic acid sequence.

SEQ ID NO: 128 is a B. licheniformis yvc target site 8 nucleic acid sequence.

SEQ ID NO: 129 is a B. licheniformis yvc target site 9 nucleic acid sequence.

SEQ ID NO: 130 is a B. licheniformis yvc target site 10 nucleic acid sequence.

SEQ ID NO: 131 is a B. licheniformis yvc target site 11 nucleic acid sequence.

SEQ ID NO: 132 is a B. licheniformis yvc target site 12 nucleic acid sequence.

SEQ ID NO: 133 is a B. licheniformis yvc target site 13 nucleic acid sequence.

SEQ ID NO: 134 is a B. licheniformis yvc target site 14 nucleic acid sequence.

SEQ ID NO: 135 is a B. licheniformis yvc target site 15 nucleic acid sequence.

SEQ ID NO: 136 is a B. licheniformis yvc target site 16 nucleic acid sequence.

SEQ ID NO: 137 is a B. licheniformis yvc target site 17 nucleic acid sequence.

SEQ ID NO: 138 is a B. licheniformis yvc target site 18 nucleic acid sequence.

SEQ ID NO: 139 is a B. licheniformis yvc target site 19 nucleic acid sequence.

SEQ ID NO: 140 is a nucleic acid sequence comprising a Cytophaga sp. variant #1 α-amylase expression cassette.

SEQ ID NO: 141 is a nucleic acid sequence comprising a Geobacillus stearothermophilus variant α-amylase expression cassette.

SEQ ID NO: 142 is a nucleic acid sequence comprising a Pseudomonas sp. AM1 variant α-amylase expression cassette.

SEQ ID NO: 143 is a nucleic acid sequence comprising a Cytophaga sp. variant #2 α-amylase expression cassette.

SEQ ID NO: 144 is a synthetic nucleic acid sequence comprising allele glcT1 (C199T).

### DETAILED DESCRIPTION

The present disclosure is generally related to compositions and methods for obtaining Bacillus licheniformis cells/strains having increased protein production capabilities. Certain embodiments of the disclosure are related to genetically modified Bacillus licheniformis cells/strains derived from parental B. licheniformis cells/strains comprising a variant rghR2 gene. Thus, certain other embodiments of the disclosure are related to modified B. licheniformis cells derived from parental B. licheniformis cells comprising a chromosomal rghR2 gene (variant) encoding a RghR2 protein of SEQ ID NO: 4, wherein the modified cells comprise a genetic modification of the rghR2 gene which encodes a RghR2 protein of SEQ ID NO: 2. Certain other embodiments of the disclosure are related to modified B. licheniformis cells derived from parental B. licheniformis cells comprising a rghR2 gene encoding a RghR2 protein comprising 90% sequence identity to SEQ ID NO: 4, wherein the modified cells comprise a genetic modification of the rghR2 gene which encodes a RghR2 protein comprising 90% sequence identity to SEQ ID NO: 2, wherein the modified cells produce an increased amount of a protein of interest (i.e., relative to the unmodified parental cells).

In other embodiments, the disclosure is related to modified B. licheniformis cells derived from parental B. licheniformis cells comprising a rghR2 gene encoding a RghR2 protein comprising 90% sequence identity to SEQ ID NO: 4, wherein the modified cells comprise a polynucleotide construct introduced therein comprising a 5′ promoter region operably linked to a nucleic acid sequence encoding a RghR2 protein comprising 90% sequence identity to SEQ ID NO: 2, wherein the modified cells produce an increased amount of a protein of interest (relative to the unmodified parental cells).

In other embodiments, the disclosure is related to modified B. licheniformis cells derived from parental B. licheniformis cells comprising a rghR2 gene encoding a RghR2 protein comprising 90% sequence identity to SEQ ID NO: 2, wherein the modified cell comprises a genetic modification which deletes, disrupts, inactivates or down-regulates at least one endogenous B. licheniformis gene selected from yvzC, Bli03644, AbrB1 and abh (AbrB2), wherein the modified cells produce an increased amount of a protein of interest (relative to the unmodified parental cells).

In other embodiments, the disclosure is related to modified B. licheniformis cells derived from parental B. licheniformis cells comprising a rghR2 gene encoding a RghR2 protein comprising 90% sequence identity to SEQ ID NO: 4, wherein the modified cell comprises a genetic modification which deletes, disrupts, inactivates or down-regulates at least one endogenous B. licheniformis gene selected from yvzC, Bli03644, AbrB1 and abh (AbrB2), wherein the modified cells produce an increased amount of a protein of interest (relative to the unmodified parental cells).

In certain other embodiments, the disclosure is related to modified B. licheniformis cells derived from parental B. licheniformis cells comprising a rghR2 gene encoding a RghR2 protein comprising 90% sequence identity to SEQ ID NO: 4, wherein the modified cells comprise a genetic modification which deletes the 18-nucleotide (18-bp) duplication in the rghR2 gene.

In other embodiments, the disclosure is related to modified B. licheniformis cells derived from parental B. licheniformis cells comprising a rghR2 gene encoding a RghR2 protein comprising 90% sequence identity to SEQ ID NO: 4, wherein the modified cells comprise a genetic modification which deletes, disrupts, inactivates or down-regulates the rghR2 gene.

In other embodiments, the disclosure is related to a modified B. licheniformis cell derived from a parental B. licheniformis cell, wherein the modified cell comprises a genetic modification which deletes, disrupts, inactivates or down-regulates at least one endogenous B. licheniformis gene encoding a YvzC protein (SEQ ID NO: 18), a BLi03644 protein (SEQ ID NO: 20), an AbrB1 protein (SEQ ID NO: 22) and/or an Abh (AbrB2) protein (SEQ ID NO: 24), wherein the modified cell produces an increased amount of a protein of interest (relative to the unmodified parental cell).

In other embodiments, a modified B. licheniformis cell derived from a parental B. licheniformis cell comprises a restored rghR2 gene of SEQ ID NO: 2 and a genetic modification which deletes, disrupts, inactivates or down-regulates at least one endogenous B. licheniformis gene encoding a YvzC protein (SEQ ID NO: 18), a BLi03644 protein (SEQ ID NO: 20), an AbrB1 protein (SEQ ID NO: 22) and/or an Abh (AbrB2) protein (SEQ ID NO: 24), wherein the modified cell produces an increased amount of a protein of interest (relative to the unmodified parental cell).

In certain other embodiments, modified B. licheniformis cells derived from parental B. licheniformis cells, comprise a rghR2ᵣₑₛₜ gene and a nucleic acid construct (SEQ ID NO: 143) comprising allele glcT1 (C199T), encoding a variant GlcT (transcriptional anti-termination) protein comprising a leucine (L) to phenylalanine (F) substitution at amino acid position 67 (L67F) of the variant GlcT protein.

Other embodiments of the disclosure are related to methods for restoring the activity of inactive RghR2 proteins in parental B. licheniformis cells. Certain other embodiments of the disclosure are related to such compositions and methods for increasing the production of proteins of interest in modified B. licheniformis cells. In other embodiments, the disclosure is related to isolated B. licheniformis (daughter) cells modified and produced by the methods of the disclosure.

### I. Definitions

In view of the modified B. licheniformis cells of the disclosure and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

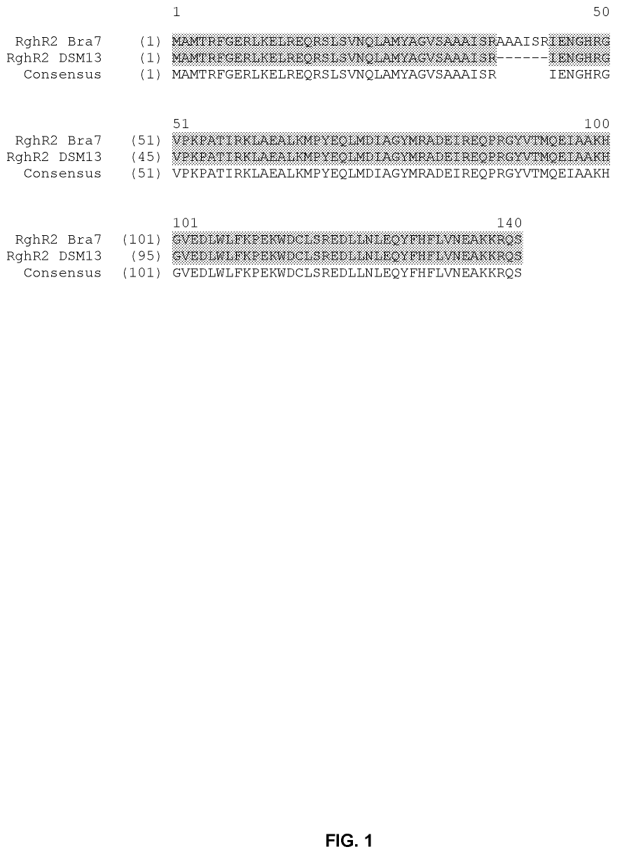

As used herein, a “native Bacillus licheniformis chromosomal rghR2 gene” comprises a nucleotide sequence encoding a RghR2 protein of SEQ ID NO: 2 (e.g., see TABLE 1 and FIG. 1).

As used herein, a “variant-18-BP” B. licheniformis chromosomal rghR2 gene comprises a nucleotide sequence encoding a variant RghR2 protein of SEQ ID NO: 4 (e.g., see TABLE 1 and FIG. 1).

As used herein, a “variant-18-BP B. licheniformis chromosomal rghR2 gene” comprising a nucleotide sequence encoding a variant RghR2 protein of SEQ ID NO: 4 may be abbreviated as “rghR2_{dup}” and the variant RghR2 protein thereof (comprising the six amino acid repeat “AAASIR”) may be abbreviated as “RghR2_{dup}”.

A used herein, a “variant B. licheniformis chromosomal rghR2 gene” includes any B. licheniformis chromosomal rghR2 gene encoding a variant RghR2 protein comprising one or more nucleotide insertions, one or more nucleotide deletions, one or more nucleotide duplications and/or one or more nucleotide substitutions in the helix-turn-helix (HTH) domain of the encoded RghR2 protein, which one or more nucleotide insertions, nucleotide deletions, nucleotide duplications and/or nucleotide substitutions in the HTH domain of the encoded RghR2 protein substantially inactivate the RghR2 protein as a transcriptional regulatory protein.

As defined herein, the “HTH domain” of the native B. licheniformis RghR2 protein of SEQ ID NO: 2 is comprised within amino residues 5-58 of SEQ ID NO: 2.

For example, in certain embodiments, one skilled in the art may readily screen/sequence rghR2 genes against the native rghR2 gene sequence of SEQ ID NO: 1, and identify “variant B. licheniformis chromosomal rghR2 gene” sequences encoding variant RghR2 proteins comprising mutations (e.g., nucleotide insertions, deletions, substitutions, duplications, and the like) in the HTH domain of the encoded RghR2 protein.

Thus, in certain embodiments, the disclosure is related to parental B. licheniformis cells comprising a “variant B. licheniformis chromosomal rghR2 gene” sequence (i.e., encoding a variant RghR2 protein comprising a mutation in the HTH domain). In other embodiments, the disclosure is related to a modified B. licheniformis cell derived from a parental cell B. licheniformis comprising a “variant B. licheniformis chromosomal rghR2 gene” sequence (i.e., encoding a variant RghR2 protein comprising a mutation in the HTH domain), wherein the modified cell comprises a restored rghR2 gene encoding a native RghR2 protein of SEQ ID NO: 2.

As used herein, a modified B. licheniformis cell derived from a parental B. licheniformis cell comprising either (i) a “variant-18-BP B. licheniformis chromosomal rghR2 gene” (SEQ ID NO: 3) or (ii) a “variant B. licheniformis chromosomal rghR2 gene” (i.e., comprising a mutation in the HTH domain of the encoded RghR2 protein comprised within amino residues 5-58 of SEQ ID NO: 2), wherein the modified B. licheniformis cell comprises a restored rghR2 gene encoding a native RghR2 protein of SEQ ID NO: 2, the “restored rghR2 gene in the modified cell” may be abbreviated herein as “rghR2ᵣₑₛₜ” and the encoded native protein thereof may be abbreviated as “RghR2ᵣₑₛₜ”.

TABLE 1
RghR2 NATIVE AND RghR2_{dup} PROTEIN SEQUENCES

| SEQ | RghR2 AMINO ACID SEQUENCE |
|---|---|
| 2 | MAMTRFGERLKELREQRSLSVNQLAMYAGVSA₃₂A₃₃A₃₄I₃₅S₃₆R₃₇ |
| | IENGHRGVPKPATIRKLAEALKMPYEQLMDIAGYMRADEIREQP |
| | RGYVTMQEIAAKHGVEDLWLFKPEKWDCLSREDLLNLEQYFHFL |
| | VNEAKKRQS |
| 4 | MAMTRFGERLKELREQRSLSVNQLAMYAGVSA₃₂A₃₃A₃₄I₃₅S₃₆ |
| | R₃₇A₃₈A₃₉A₄₀I₄₁S₄₂R₄₃IENGHRGVPKPATIRKLAEALKMPYEQL |
| | MDIAGYMRADEIREQPRGYVTMQEIAAKHGVEDLWLFKPEKWDC |
| | LSREDLLNLEQYFHFLVNEAKKRQS |

More specifically, as presented above in TABLE 1, a “variant-18-BP B. licheniformis chromosomal rghR2 gene” of the disclosure (SEQ ID NO: 3) comprises an 18-nucleotide (18-bp) duplication encoding a consecutive repeat of six (6) amino acids which are “Ala-Ala-Ala-Ile-Ser-Arg” (hereinafter “AAAISR”), wherein the primary (1º) amino acid sequence of the encoded variant RghR2 protein is set forth as SEQ ID NO: 4 (see, TABLE 1 and FIG. 1) comprises a linear (consecutive) repeat of these six (6) amino acids as follows: “Ala-Ala-Ala-Ile-Ser-Arg-Ala-Ala-Ala-Ile-Ser-Arg”; hereinafter, “AAAISRAAAISR” (SEQ ID NO: 6). For example, the six amino acid repeat present in RghR2_{dup} protein (SEQ ID NO: 4) is presented in TABLE 1, wherein the repeated amino acid residues of this 140 amino acid protein comprise the bold text amino acids at positions A₃₈ to R₄₃ of SEQ ID NO: 4.

In contrast, a “native B. licheniformis chromosomal rghR2 gene” of the disclosure (SEQ ID NO: 1) does not comprise this 18-nucleotide (18-bp) duplication. Thus, the native rghR2 gene encodes the native RghR2 protein of SEQ ID NO: 2 (which does not comprise the consecutive repeat “AAAISR”, as presented in SEQ ID NO: 6). For example, the primary (1°) amino acid sequence of the encoded native RghR2 protein is presented in TABLE 1, wherein the six amino acid repeat of “AAAISR” is not present at positions 38-43 (SEQ ID NO: 2) of this 134 amino acid protein.

As defined herein, a B. licheniformis strain Bra7 (or Bra7 strain) is a B. licheniformis host cell developed/derived from a wild-type B. licheniformis parental strain using classical genetic improvements methods. Although certain embodiments and descriptions of the present disclosure are related to B. licheniformis strain Bra7, the compositions and methods of the instant disclosure are not limited to a specific Bacillus species, nor are the compositions and methods of the instant disclosure limited to a specific strain of B. licheniformis host cells.

As used herein, a “B. licheniformis derivative of strain Bra7”, specifically refers to a B. licheniformis (daughter) cell derived from a parental B. licheniformis Bra7 (strain) host cell. More particularly, as used herein, a “B. licheniformis derivative of strain Bra7” is a B. licheniformis host cell derived from the B. licheniformis strain Bra7 parent which comprises a five (5) gene deletion (Δcat, ΔamyL, Δspo, ΔaprL, ΔendoGluC) as described in International PCT Publication No. WO2008/024372.

As used herein, a heterologous Peanibacillus curdlanolyticus variant α-amylase (e.g., see, Examples 2 and 4), optionally abbreviated herein as “PcuAmyl-v6”, is disclosed in PCT Publication No. WO2014/164834.

As used herein, a Cytophaga sp. variant α-amylase referred to herein as “Cytophaga sp. α-amylase variant #1” (e.g., see, Example 5) and “Cytophaga sp. α-amylase variant #2” (e.g., see, Examples 11 and 12) are disclosed in International PCT Publication Nos. WO2014/164777; WO2012/164800 and WO2014/164834.

As used herein, a “variant Geobacillus stearothermophilus amylase” (e.g., see, Example 5) is a variant G. stearothermophilus α-amylase disclosed in International PCT Publication No. WO2009/149130.

As used herein, a “variant alkaline α-amylases” (e.g., see, Example 9), referred to herein as alkaline α-amylase “variant 1”, alkaline α-amylase “variant 2”, alkaline α-amylase “variant 3” and alkaline α-amylase “variant 4”, which are variant α-amylase derived from Bacillus sp. No. 707 comprising improved alkaline performance/stability thereof, are generally disclosed in International PCT Publication No. WO2008/153805 and US Patent Publication No. US2014/0057324.

As used herein, a “G4 amylase (variant)” of Pseudomonas sp. AM1 (e.g., see, Example 8) is disclosed in International PCT Publication No. WO2010/133644.

As used herein, a heterologous DNA/nucleic acid sequence “encoding an enzyme comprising lipase/esterase activity” (e.g., see, Example 10), such DNA/nucleic acid sequence encodes an enzyme commission number “EC 3.1.1.3“enzyme” comprising lipase/esterase activity.

As used herein, a B. licheniformis (daughter) cell comprising allele glcT1 (e.g., see, Example 12), allele glcT1 encodes a variant GlcT (transcriptional anti-termination) protein comprising a phenylalanine (F) at amino acid position 67 (F67) of the variant GlcT protein, as described in U.S. Provisional Patent Application Serial No. U.S. 62/613,339, filed Jan. 3, 2018.

Thus, in certain embodiments, a “native B. licheniformis chromosomal rghR2 gene” comprises a nucleotide sequence encoding a RghR2 protein comprising at least 90% sequence identity to SEQ ID NO: 2. In other embodiments, a “native B. licheniformis chromosomal rghR2 gene” comprises a nucleotide sequence encoding a RghR2 protein comprising at least 90% sequence identity to SEQ ID NO: 2, with the proviso that the RghR2 protein comprising 90% sequence identity to SEQ ID NO: 2 is active as a transcriptional regulatory protein in B. licheniformis cells. In certain other embodiments, a “native B. licheniformis chromosomal rghR2 gene” comprises a nucleotide sequence encoding a RghR2 protein comprising at least 90% sequence identity to SEQ ID NO: 2, with the proviso that the RghR2 protein comprising 90% sequence identity to SEQ ID NO: 2 does not comprise a repeat of amino acids AAAISR as set forth in SEQ ID NO: 6.

In certain embodiments, a rghR2_{dup} gene comprises a nucleotide sequence encoding a RghR2 protein of SEQ ID NO: 4. In certain embodiments, a rghR2_{dup} gene comprises a nucleotide sequence encoding a RghR2 protein comprising at least 90% sequence identity to SEQ ID NO: 4. In certain embodiments, a rghR2_{dup} gene comprises a nucleotide sequence encoding a RghR2 protein comprising at least 90% sequence identity to SEQ ID NO: 4, with the proviso that the RghR2 protein comprising 90% sequence identity to SEQ ID NO: 4 is substantially inactive as a transcriptional regulatory protein in B. licheniformis cells. In certain other embodiments, a rghR2_{dup} gene comprises a nucleotide sequence encoding a RghR2 protein comprising at least 90% sequence identity to SEQ ID NO: 4, with the proviso that the RghR2 protein comprising 90% sequence identity to SEQ ID NO: 4 comprises a repeat of amino acids AAAISR as set forth in SEQ ID NO: 6.

In other embodiments, a variant rghR2 gene of the disclosure comprises any B. licheniformis rghR2 gene comprising at least one, two, three, four, five, ten, eighteen, twenty, etc. nucleotides inserted, deleted, duplicated and the like in the native rghR2 gene's nucleic acid sequence between nucleotide positions 111 and 112 of SEQ ID NO: 1, thereby converting the native rghR2 gene into a variant rghR2 gene comprising at least one, two, three, four, five, ten, eighteen, twenty, etc. nucleotides inserted into the native rghR2 gene's nucleic acid sequence.

Thus, in other embodiments, a RghR2 protein which is substantially inactive further includes variant RghR2 proteins comprising at least one, two, three, four, five, six, seven, etc. amino acids inserted between amino acid residues 37 and 38 of the RghR2 protein (i.e., with reference to the active RghR2 protein sequence of SEQ ID NO: 2), wherein such amino acid insertions (i.e., between residues 37 and 38) render the RghR2 protein substantially inactive as transcriptional regulatory protein.

Thus, in certain embodiments, a variant RghR2 protein encoded by a variant rghR2 gene is substantially “inactive as a transcriptional regulatory protein” in B. licheniformis cells.

As defined herein, an “RghR2_{dup} protein of SEQ ID NO: 4, comprising a 6-amino acid repeat of amino acids AAAISR” is substantially “inactive as transcriptional regulatory protein” in B. licheniformis cells.

As defined herein, a “variant rghR2 gene encoding a variant rghR2 protein comprising a mutation in the HTH domain” of the encoded RghR2 of SEQ ID NO: 2 is substantially “inactive as transcriptional regulatory protein” in B. licheniformis cells relative to the native RghR2 protein of SEQ ID NO: 2.

In certain embodiments, a rghR2 gene encoding a variant RghR2 protein which is substantially “inactive as a transcriptional regulatory protein” is determined by screening variant RghR2 proteins (relative to native RghR2 protein; SEQ ID NO: 2) in DNA binding assays known to one skilled in the art.

For example, it is contemplated herein that the RghR2 protein, a transcriptional regulatory protein comprising the HTH domain set forth above, must sufficiently bind to DNA to exert its transcriptional regulatory activity thereof. Thus, by comparing DNA binding affinities of the native RghR2 protein relative to one or more variant RghR2 proteins (i.e., comprising a mutated HTH domain), a reduced DNA binding affinity of a variant RghR2 protein vis-à-vis the native RghR2 protein serves as a corollary for such variant RghR2 proteins which are substantially “inactive as transcriptional regulatory proteins”. For example, as contemplated herein, a variant RghR2 protein (i.e. comprising a mutated HTH domain) having a significantly reduced DNA binding affinity (or a complete loss of DNA binding) will have a substantial reduction (or complete loss) of transcriptional regulatory protein activity, relative to the native RghR2 protein, which is substantially active as transcriptional regulatory protein.

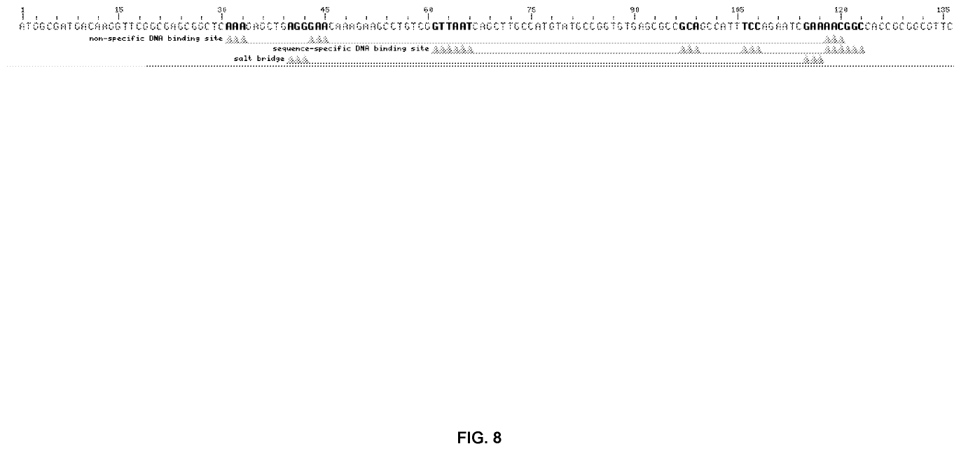

Thus, in certain embodiments, a variant rghR2 gene of the disclosure includes any B. licheniformis rghR2 gene variant comprising an insertion, duplication, deletion, non-synonymous substitution, and the like, of the nucleotides in these (DNA binding) regions of the rghR2 gene, as presented in FIG. 8. For example, FIG. 8 of the instant disclosure shows certain rghR2 codons (i.e., the bold text nucleotides in FIG. 8) predicted to encode amino acid residues in the RghR2 protein involved in DNA binding. Thus, in certain embodiments, a variant rghR2 gene of the disclosure is a variant rghR2 gene comprising an insertion, duplication, deletion, non-synonymous substitution, and the like of one or more nucleotides in these (DNA binding) regions of the rghR2 gene. More particularly, in certain embodiments, a variant rghR2 gene comprising an insertion, duplication, deletion, non-synonymous substitution, and the like of the nucleotides in the DNA binding regions of the rghR2 gene encodes a substantially inactive RghR2 protein.

The phrase a RghR2 protein which is substantially “inactive as a transcriptional regulatory protein” includes variant RghR2 proteins encoded by variant rghR2 genes comprising an insertion, duplication, deletion, non-synonymous substitution (and the like) of one or more nucleotides in these (DNA binding) regions of the rghR2 gene, wherein the encoded variant proteins are substantially inactive as transcriptional regulatory proteins.

As used herein, the term “equivalent positions” mean the amino acid residue positions after alignment with the RghR2 polypeptide sequence of SEQ ID NO: 4, particularly from amino acid residues 32 to 43 of SEQ ID NO: 4. The twelve (12) contiguous amino acids residues (i.e., equivalent positions) described above for SEQ ID NO: 4 (i.e., residues 32 to 43 of SEQ ID NO: 4) are presented in the amino acid sequence of SEQ ID NO: 6 (“AAAISRAAAISR). Thus, in certain embodiments, a gene or ORF encoding a variant RghR2 protein of the disclosure may be identified by comparison of the equivalent positions of the encoded RghR2 protein's amino acid sequence to the repeat amino acid sequence set forth in SEQ ID NO: 6, wherein the presence of the SEQ ID NO: 6 repeat sequence indicates a variant RghR2 protein of the disclosure.

The terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein. For example, as used herein a genetic modification includes, but is not limited to, a modification of one or more genes selected from the group consisting of rghR2, rghR1, abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, spo0F, ywjG, ywq12, BLi04199, BLi04200, licT, bg1H and bg1P.

As used herein, “disruption of a gene”, “gene disruption”, “inactivation of a gene” and “gene inactivation” are used interchangeably and refer broadly to any genetic modification that substantially prevents a host cell from producing a functional gene product (e.g., a protein). Exemplary methods of gene disruptions include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and any combinations and variations thereof which disrupt/inactivate the target gene(s) and substantially reduce or prevent the production of the functional gene product (i.e., a protein).

As used herein, the terms “down-regulation” of gene expression and “up-regulation” of gene expression include any method that results in lower (down-regulated) or higher (up-regulated) expression of a gene. For example, the down-regulation of a gene can be achieved by RNA-induced gene silencing, genetic modifications of control elements such as the promoter, ribosomal binding site (RBS)/Shine-Dalgarno sequences, untranslated regions (UTRs), codon changes, and the like.

As used herein, the phrases “deleting the 18-nucleotide duplication”, or “deleting the 18-bp duplication” or “modifying the cell by deleting the 18-nucleotide duplication” particularly refer to a genetic modification of a parental Bacillus cell comprising a variant rghR2 gene comprising an 18-nucleotide duplication (rghR2_{dup}), which duplication encodes a repeat of amino acids “AAAISR” (SEQ ID NO: 6) in the variant RghR2 protein (RghR2_{dup}; e.g., see SEQ ID NO: 4, wherein amino acids “AAAISR” at positions 32-37 of SEQ ID NO: 4 are consecutively repeated at positions 38-43 of SEQ ID NO: 4).

Thus, in certain embodiments, a modified Bacillus cell of the disclosure is derived from a parental Bacillus cell comprising a variant chromosomal rghR2 gene (e.g., rghR2_{dup}; SEQ ID NO: 3) comprising an 18-nucleotide duplication encoding the “AAAISR” repeated sequence of SEQ ID NO: 6, wherein the modified Bacillus cell is modified by “deleting the 18-nucleotide duplication”, thereby resulting in a modified Bacillus cell comprising a “restored” rghR2 gene sequence (rghR2ᵣₑₛₜ; SEQ ID NO: 1) encoding a native rghR2 protein. Methods for deleting the 18-nuclotide duplication in the parental Bacillus cell include, but are not limited to, homologous recombination, CRSIPR-Cas9 gene editing, mega-nuclease gene editing, TALEN gene editing, Zinc-Finger Nuclease (ZFN) editing and the like, which are further described below in Section IV. Thus, in certain embodiments, the disclosure is directed to modified Bacillus cells comprising a “restored” rghR2 gene”.

As used herein, “host cell” refers to a cell that has the capacity to act as a host or expression vehicle for a newly introduced DNA sequence. This, in certain embodiments of the disclosure, the host cells are Bacillus sp. or E. coli cells.

As defined herein, a “parental cell”, a “parental host cell” or a “parental B. licheniformis (host) cell”, may be used interchangeably and refer to “unmodified” parental B. licheniformis cells. For example, a “parental” cell refers to any cell or strain of microorganism in which the genome of the “parental” cell is altered (e.g., via one or more mutations introduced into the parental cell) to generate a modified “daughter” cell.

As defined herein, a “modified cell”, a “modified host cell” or a “modified B. licheniformis (host) cell”, may be used interchangeably and refer to recombinant B. licheniformis (host) cells that comprise at least one genetic modification which is not present in the “parental” B. licheniformis host cell from which the modified B. licheniformis (daughter) cell is derived. For example, in certain embodiments a “parental B. licheniformis (host) cell” of the disclosure comprises a chromosomal rghR2 gene of SEQ ID NO: 3 (rghR2_{dup}) encoding a variant RghR2 protein of SEQ ID NO: 4, and a “modified” B. licheniformis host cell the disclosure (i.e., derived from the parental B. licheniformis host cell comprising the chromosomal rghR2 gene of SEQ ID NO: 3) comprises a genetic modification which deletes the 18-nucleotide duplication in SEQ ID NO: 3, thereby resulting in a modified (restored) B. licheniformis host cell comprising a native rghR2 gene (rghR2ᵣₑₛₜ) encoding a native RghR2 protein of SEQ ID NO: 2.

In certain embodiments, the “unmodified” B. licheniformis (parental) cell may be referred to as a “control cell”, particularly when being compared with, or relative to, a “modified” B. licheniformis (daughter) cell. As used herein, when the expression and/or production of a protein of interest (POI) in an “unmodified” (parental) cell (i.e., a control cell) is being compared to the expression and/or production of the same POI in a “modified” (daughter) cell, it will be understood that the “modified” and “unmodified” cells are grown/cultivated/fermented under the same conditions (e.g., the same conditions such as media, temperature, pH and the like).

As used herein, “the genus Bacillus” includes all species within the genus “Bacillus”’ as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As defined herein, the terms “increased expression”, “enhanced expression”, “increased expression of a POI”, “increased production”, “increased production of a POI” and the like refer to a “modified” B. licheniformis (daughter) cell, wherein the “increase” is always relative (vis-à-vis) to an “unmodified” B. licheniformis (parental) cell expressing/producing the same POI.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any step involved in the production of the polypeptide including, but not limited to, transcription, post-transcriptional modification, translation, post-translational modification, secretion and the like.

As defined herein, the combined term “expresses/produces”, as used in phrases such as “a modified host cell expresses/produces an increased amount of a protein of interest relative to the (unmodified) parental host cell”, the term (“expresses/produces”) is meant to include any steps involved in the expression and production of a protein of interest in host cell of the disclosure.

Likewise, as used herein, an “increased amount”, when used in phrases such as “a modified host cell ‘expresses/produces an increased amount’ of one or more proteins of interest relative to the (unmodified) parental host cell”, particularly refers to an “increased amount” of any protein of interest (POI) expressed/produced in the modified host cell, which “increased amount” is always relative to the (unmodified) parental B. licheniformis cells expressing/producing the same POI, wherein the modified and unmodified cells are grown/cultured/fermented under the same conditions (e.g., the same conditions such as media, temperature, pH and the like). For example, an increased amount of a POI may be an endogenous B. licheniformis POI or a heterologous POI expressed in a modified B. licheniformis cell of the disclosure.

Thus, as used herein, “increasing” protein production or “increased” protein production is meant an increased amount of protein produced (e.g., a protein of interest). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as protease activity, amylase activity, cellulase activity, hemicellulase activity and the like), or total extracellular protein produced as compared to the parental host cell.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) or more codons representing amino acids, and (iii) a termination codon, the ORF being read (or translated) in the 5′ to 3′ direction.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence encoding an RghR2 protein of SEQ ID NO: 2. Thus, in certain embodiments, a functional promoter sequence controls the expression of an rghR2 gene encoding a RghR2 protein of SEQ ID NO: 2. In other embodiments, a functional promoter sequence controls the expression of a heterologous gene (or endogenous gene) encoding a protein of interest in a Bacillus cell, more particularly in a B. licheniformis host cell.

As defined herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As defined herein, the term “introducing”, as used in phrases such as “introducing into a bacterial cell” or “introducing into a B. licheniformis cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like (e.g., see Ferrari et al., 1989).

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). For example, in certain embodiments of the disclosure, a parental B. licheniformis cell comprising a variant rghR2 gene encoding a variant RghR2 protein of SEQ ID NO: 4 is modified (e.g., transformed) by introducing into the parental cell a polynucleotide construct comprising a promoter operably linked to a nucleic acid sequence encoding a native RghR2 protein of SEQ ID NO: 2, thereby resulting in a modified B. licheniformis (daughter) host cell derived from the “unmodified” (parental) cell.

As used herein, “transformation” refers to introducing an exogenous DNA into a host cell so that the DNA is maintained as a chromosomal integrant or a self-replicating extra-chromosomal vector. As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction (See e.g., Ferrari et al., 1989).

As used herein “an incoming sequence” refers to a DNA sequence that is introduced into the Bacillus chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the Bacillus chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the Bacillus chromosome a DNA construct is integrated and directs what part of the Bacillus chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

In still another embodiment of the disclosure, the deletion, disruption, inactivation or down-regulation of a gene active at an inappropriate time, as determined by DNA array analysis (e.g., transcriptome analysis, as described herein) provides enhanced expression of a protein of interest. As used herein, “transcriptome analysis” refers to the analysis of gene transcription.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, spec^{R}, kan^{R}, ery^{R}, tet^{R}, cmp^{R} and neo^{R} (see e.g., Guerot-Fleury, 1995; Palmeros et al., 2000; and Trieu-Cuot et al., 1983). In some embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids (See e.g., Albertini and Galizzi, 1985; Stahl and Ferrari, 1984). Other markers useful in accordance with the invention include, but are not limited to auxotrophic markers, such as serine, lysine, tryptophan; and detection markers, such as β-galactosidase.

As defined herein, a host cell “genome”, a bacterial (host) cell “genome”, or a B. licheniformis (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental B. licheniformis (host) cell comprising a variant rghR2 gene encoding a variant RghR2 protein of SEQ ID NO: 4, is modified (e.g., transformed) by introducing into the parental cell one or more “targeting vectors” which are designed to delete the 18-nucleotde duplication of the endogenous B. licheniformis variant rghR2 gene, such that modified host cell comprising the “restored” native rghR2 gene encodes a native RghR2 protein of SEQ ID NO: 2. Selection and/or construction of appropriate vectors (e.g., for the deletion of the 18-nucleotide duplication in the rghR2 gene) is well within the knowledge of those having skill in the art.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

As used herein, the term “protein of interest” or “POI” refers to a polypeptide of interest that is desired to be expressed in a modified B. licheniformis (daughter) host cell, wherein the POI is preferably expressed at increased levels (i.e., relative to the “unmodified” (parental) cell). Thus, as used herein, a POI may be an enzyme, a substrate-binding protein, a surface-active protein, a structural protein, a receptor protein, and the like. In certain embodiments, a modified cell of the disclosure produces an increased amount of a heterologous protein of interest or an endogenous protein of interest relative to the parental cell. In particular embodiments, an increased amount of a protein of interest produced by a modified cell of the disclosure is at least a 0.5% increase, at least a 1.0% increase, at least a 5.0% increase, or a greater than 5.0% increase, relative to the parental cell.

Similarly, as defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous” nucleic acid construct or a “heterologous” nucleic acid sequence has a portion of the sequence which is not native to the cell in which it is expressed.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to the another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wisconsin, USA 53711) (Needleman and Wunsch, (1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, the term “ComK polypeptide” is defined as the product of a comK gene; a transcription factor that acts as the final auto-regulatory control switch prior to competence development; involved with activation of the expression of late competence genes involved in DNA-binding and uptake and in recombination (Liu and Zuber, 1998, Hamoen et al., 1998). Exemplary ComK nucleic acid and polypeptide sequences are set forth in SEQ ID NO: 85 and SEQ ID NO: 86, respectively.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, “orthologue” and “orthologous genes” refer to genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “homology” refers to sequence similarity or identity, with identity being preferred. This homology is determined using standard techniques known in the art (See e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, WI) and Devereux et. al., 1984).

As used herein, an “analogous sequence” is one wherein the function of the gene is essentially the same as the gene derived from a Bacillus licheniformis cell. Additionally, analogous genes include at least 60%, 65%, 70%, 75%, 80%, 85%, 90%, 95%, 97%, 98%, 99% or 100% sequence identity with the sequence of the Bacillus licheniformis cell. Analogous sequences are determined by known methods of sequence alignment. A commonly used alignment method is BLAST, although there are other methods that also find use in aligning sequences.

As used herein, the term “hybridization” refers to the process by which a strand of nucleic acid joins with a complementary strand through base pairing, as known in the art. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tₘ) of the nucleic acid binding complex or probe. For example, “maximum stringency” typically occurs at about Tₘ ⁻5° C. (5° below the Tₘ of the probe); “high stringency” at about 5-10° C. below the Tₘ; “intermediate stringency” at about 10-20° C. below the Tₘ of the probe; and “low stringency” at about 20-25° C. below the Tₘ. Functionally, maximum stringency conditions may be used to identify sequences having strict identity or near-strict identity with the hybridization probe; while an intermediate or low stringency hybridization can be used to identify or detect polynucleotide sequence homologs. Moderate and high stringency hybridization conditions are well known in the art. An example of high stringency conditions includes hybridization at about 42° C. in 50% formamide, 5×SSC, 5×Denhardt's solution, 0.5% SDS and 100 pg/ml denatured carrier DNA, followed by washing two times in 2×SSC and 0.5% SDS at room temperature (RT) and two additional times in 0. 1×SSC and 0.5% SDS at 42° C. An example of moderate stringent conditions including overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denaturated sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. “Recombination”, “recombining” or generating a “recombined” nucleic acid is generally the assembly of two or more nucleic acid fragments wherein the assembly gives rise to a chimeric gene.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

As used herein, the term “stuffer sequence” refers to any extra DNA that flanks homology boxes (typically vector sequences). However, the term encompasses any non-homologous DNA sequence. Not to be limited by any theory, a stuffer sequence provides a non-critical target for a cell to initiate DNA uptake.

### II. B. Licheniformis RghR1/RghR2 Transcriptional Regulators

The Bacillus subtilis yvaN gene was identified as a repressor of rapG, rapH (Hayashi et al., 2006) and rapD (Ogura & Fujita, 2007), and renamed rghR (rapG and rapH Repressor). Downstream of rghR lies a gene yvaO, with an unknown function, but based on sequence homology encodes a HTH-type (helix-turn-helix) transcriptional regulator. The amino acid sequence identity between “RghR” and “YvaO” is approximately 52%. Upstream of the B. subtilis rghR are the genes yvzC and yvaM. The yvzC gene also encodes a putative HTH-type transcriptional regulator, while the translation product of yvaM is a putative hydrolase.

More particularly, Bacillus licheniformis encodes two homologs of Bacillus subtilis RghR/YvaO, which are named “RghR1” and “RghR2”. The amino acid sequence identity between the B. subtilis (strain 168) “RghR” protein and B. licheniformis (strain DSM 13) “RghR1” protein is approximately 59%; and the amino acid sequence identity between B. subtilis (strain 168) “RghR” and B. licheniformis (strain DSM 13) “RghR2” is approximately 57%.

Upstream of the B. licheniformis rghR1 are two genes, yvzC (BLi03645; SEQ ID NO: 17) and BLi03644 (SEQ ID NO: 19) transcriptional regulators. The B. licheniformis YvzC is a homolog of B. subtilis YvzC. However, Bli03644 belongs to the AbrB family of transcriptional regulators, and is not a homolog of the putative hydrolase YvaM.

More particularly, as presented and discussed in the Examples section below, B. licheniformis (strain DSM13=ATTC 14580) contains a gene, designated rghR2 (KEGG Genome T00200 B. licheniformis DSM13 Gene ID No. BLi03647), encoding a putative HTH-type transcriptional regulator. The nucleic acid sequence of rghR2 of B. licheniformis (DSM13) is presented in SEQ ID NO: 1 and the encoded amino acid sequence of the RghR2 protein of Bacillus licheniformis (DSM13) is presented in SEQ ID NO: 2.

For example, Applicant of the present disclosure sequenced (1) the genome of B. licheniformis strain Bra7, (2) the genome of a B. licheniformis derivative of strain Bra7, (3) the genome of B. licheniformis strain ATCC-9789 (www.atcc.org/Products/All/9789.aspx), and (4) the genome of B. licheniformis strain ATCC-6598 (www.atcc.org/en/Products/All/6598.aspx), which revealed that all of these B. licheniformis strains have a duplication (i.e., a repeat) of 18 nucleotides (18-bp) in the rghR2 gene (e.g., see SEQ ID NO: 3, wherein nucleotides “GCCGCAGCCATTTCCAGA” are repeated twice in consecutive order, which nucleotide duplication is set forth in SEQ ID NO: 87) and wherein this 18-nucleotide sequence encodes amino acids “AAAISR” (SEQ ID NO: 5) such that the variant RghR2 protein of SEQ ID NO: 4 comprises a repeat of “AAAISR” (i.e., AAAISR-AAAISR; as presented in SEQ ID NO: 6).

Thus, the amino acid sequence of the RghR2 protein of the B. licheniformis Bra7 strain (SEQ ID NO: 4) is identical to the sequence of RghR2 of the B. licheniformis Bra7 derivative, the B. licheniformis strain ATCC-9789 and the B. licheniformis strain ATCC-6598, as presented in SEQ ID NO: 4.

An alignment of the B. licheniformis Bra7 strain RghR2 protein amino acid sequence (SEQ ID NO: 4) and the B. licheniformis DSM13 strain RghR2 protein amino acid sequence (SEQ ID NO: 2) is presented FIG. 1, illustrating the repeat (AAAISR) in SEQ ID NO: 4. The insertion (repeat) of the sequence “AAAISR” is in the helix-turn-helix (HTH) domain and near the sequence-specific DNA-binding site of the RghR2 protein as shown in FIG. 8.

For example, a RghR2 protein of SEQ ID NO: 4 (e.g., encoded by a rghR2_{dup} gene) comprises 140 amino acid residues, a molecular weight of ˜16.1 kDa, a theoretical net charge of ⁺4.5 and theoretical isoelectric point (P_{I}) of 9.38 (molecular weight, net charge and P_{I} calculation based on 1º amino acid sequence), whereas a RghR2 protein of SEQ ID NO: 2 (e.g., encoded by a native or rghR2ᵣₑₛₜ gene) comprises 134 amino acid residues, a molecular weight of ˜15.6 kDa, a theoretical net charge of ⁺3.5 and a theoretical isoelectric point (P_{I}) of 8.85 (molecular weight, net charge and P_{I} calculation based on 1º amino acid sequence). Likewise, assessment of RghR2 protein sequence using Pfam analysis (version 31.0) indicates that the Rgh2 protein comprises a helix-turn-helix (HTH) domain of HTH family_31 (HTH_31; clan-0123), which HTH domain is comprised within amino residues 5-58 of the RghR2 protein of SEQ ID NO: 2. For example, the rghR2 gene of the B. licheniformis Bra7 strain, encoding the variant RghR2 protein of SEQ ID NO: 4, comprises a duplication of the six amino acid repeat “AAAISR” (FIG. 1), which six amino acid repeat is located approximately in the middle of the HTH domain (amino acid) sequence.

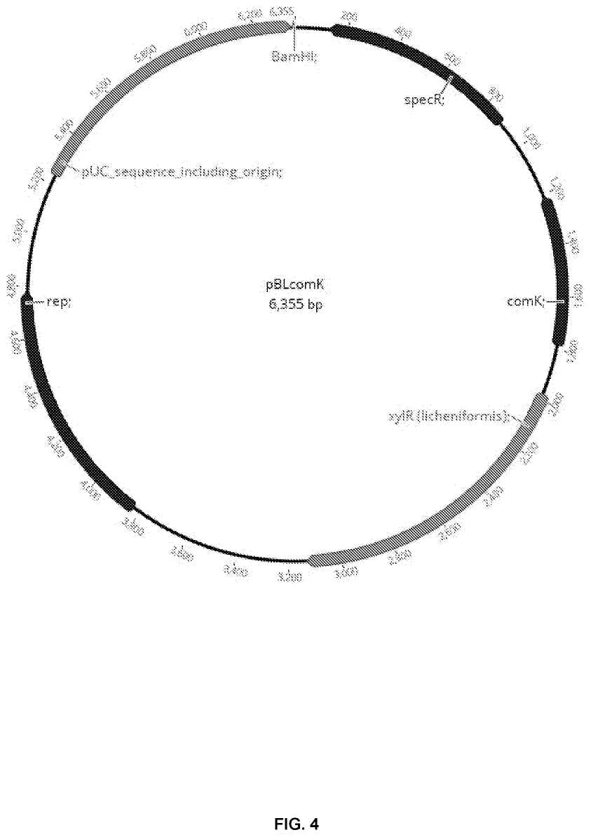

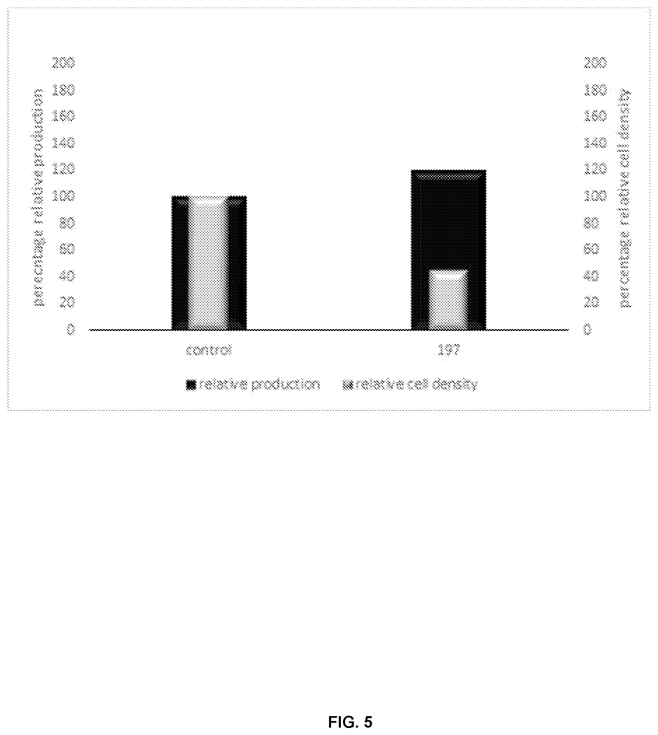

Without wishing to be bound by a particular theory, mechanism or mode of operation, it is contemplated herein that the insertion (repeat) of the sequence “AAAISR” in SEQ ID NO: 4, significantly affects, or even completely abolishes, the function of the RghR2 protein as a transcriptional regulator. For example, as a transcription regulator, RghR2 will directly and indirectly regulate the expression of several other genes (e.g., see Example 3). Thus, inactivation of RghR2 by this 18-bp nucleotide duplication encoding the “AAAISR” amino acid repeat set forth in SEQ ID NO: 6 is contemplated to affect the physiology of the cell and as such, may impact factors like cell growth and heterologous protein production. More particularly, the impact of this 18-bp nucleotide duplication present in SEQ ID NO: 3 was further studied in the Example 2, by removing (e.g., deleting) the 18-bp duplication in the rghR2 gene in the B. licheniformis derivative of Bra7 (strain) cells producing various heterologous enzymes. More particularly, as presented in FIG. 4, deletion of the rhgR2 18-bp duplication showed a decrease in biomass when cultured, but at the same time demonstrated an improved amylase production titer (i.e., increased production of a protein of interest). Thus, as presented in FIG. 5, the specific productivity (enzyme production/OD₆₀₀) improved by at least a factor 2 in the rghR2 restored (i.e., Δ18-bp duplication) strain.

### III. Transcriptome Analysis of Genes Up-Regulated and Down-Regulated in B. Licheniformis rghR2 Variant and rghR2 Restored Cells

As set forth in Example 3, transcriptome analysis of the B. licheniformis derivative of Bra7 strain cells (i.e., comprising rghR2 with the 18-bp duplication; SEQ ID NO: 3) and the rghR2 restored variant of this strain (i.e., via removal of the 18-bp duplication; SEQ ID NO: 1), revealed that the transcription of several genes are regulated by RghR2. For example, the transcription of genes upregulated and downregulated by at least two-fold in the rghR2 restored strain (i.e., relative to the rghR2 inactive strain comprising the 18-bp duplication) are indicated in Example 3, TABLE 2 and TABLE 3, respectively.

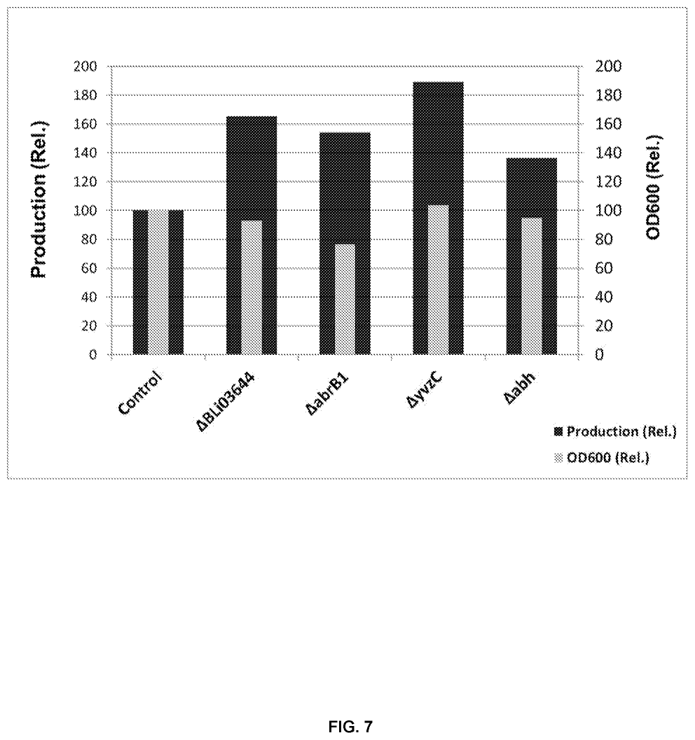

More particularly, it is contemplated herein that the deletion, disruption, inactivation or down-regulation of one or more the genes in TABLE 3, in either rghR2 restored B. licheniformis strains (i.e., comprising the rghR2 gene encoding the RghR2 protein of SEQ ID NO: 2) or rghR2 inactivated B. licheniformis strains (i.e., comprising the rghR2 gene encoding the RghR2 protein of SEQ ID NO: 4) will have a positive effect on protein production in these modified host cells, similar to the effect observed by re-activation of rghR2 gene (i.e., via removal/deletion of the 18-bp repeat in the rghR2 gene). Thus, as presented in Example 4, the effect of inactivation (e.g., a deletion, disruption or down-regulation) of a subset of these genes (i.e., Bli03644 (SEQ ID NO: 19); yvzC (SEQ ID NO: 17); abrB1 (SEQ ID NO: 21) and abh (SEQ ID NO: 23)) on heterologous protein production was explored. For example, the Bli03644, abrB1, yvzC and abh genes were inactivated by insertion of antibiotic marker in a B. licheniformis Bra7 derivative producing a heterologous α-amylase. Thus, the amylase production was determined in four single knock-out strains (i.e., ΔBLi03644, ΔabrB1, ΔyvzC and Δabh) and compared to the parental strain as control (as described in Example 2). More particularly, as presented in FIG. 7, inactivation of Bli03644, abrB1, yvzC and abh resulted in improved α-amylase production, while cell growth (OD₆₀₀) was less affected (i.e., demonstrating an increased specific productivity, Qp).

### IV. Molecular Biology

As set forth above, certain embodiments of the disclosure are related to modified B. licheniformis cells derived from parental B. licheniformis cells comprising a rghR2 gene encoding a RghR2 protein comprising 90% sequence identity to SEQ ID NO: 4, wherein the modified cells comprise a genetic modification of the rghR2 gene which encodes a RghR2 protein comprising 90% sequence identity to SEQ ID NO: 2. In other embodiments the disclosure is related to modified B. licheniformis cells derived from parental B. licheniformis cells comprising a rghR2 gene encoding a RghR2 protein of SEQ ID NO: 4, wherein the modified cells comprise a restored rghR2 gene encoding a RghR2 protein of SEQ ID NO: 2. In another embodiment the disclosure is related to modified B. licheniformis cells derived from parental B. licheniformis cells comprising a rghR2 gene encoding a RghR2 protein comprising 90% sequence identity to SEQ ID NO: 2 and a genetic modification which deletes, disrupts, inactivates or down-regulates at least one endogenous B. licheniformis gene selected from the group consisting of abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, rghR1, spo0F, ywjG, ywq12, BLi04199, BLi04200, licT, bg1H and bg1P. In another embodiment the disclosure is related to modified B. licheniformis cells derived from parental B. licheniformis cells comprising a rghR2 gene encoding a RghR2 protein comprising 90% sequence identity to SEQ ID NO: 4 and a genetic modification which deletes, disrupts, inactivates or down-regulates at least one endogenous B. licheniformis gene selected from the group consisting of abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, rghR1, spo0F, ywjG, ywq12, BLi04199, BLi04200, licT, bg1H and bg1P. Other embodiments are related to genetic modifications which alter the coding sequence of an RghR2 protein's HTH domain.

Thus, certain embodiments of the disclosure provide compositions and methods for genetically modifying (altering) a parental B. licheniformis cell of the disclosure to generate modified (rghR2ᵣₑₛₜ) cells, and more particularly, modified B. licheniformis (rghR2ᵣₑₛₜ) cells which produce an increased amount of an endogenous or heterologous protein of interest (i.e., relative to the (unmodified) parental B. licheniformis cells).

Thus, certain embodiments of the disclosure are directed to methods for genetically modifying Bacillus cells, wherein the modification comprises, but is not limited to, (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) a gene down-regulation, (f) site specific mutagenesis and/or (g) random mutagenesis. For example, as used herein a genetic modification includes, but is not limited to, a modification of one or more genes selected from the group consisting of rghR1, rghR2, abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, spo0F, ywjG, ywq12, BLi04199, BLi04200, licT, bg1H and bg1P.

In certain embodiments, a modified Bacillus cell of the disclosure is constructed by reducing or eliminating the expression of a gene set forth above, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified Bacillus cell is constructed by gene deletion to eliminate or reduce the expression of at least one of the aforementioned genes of the disclosure. Gene deletion techniques enable the partial or complete removal of the gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a Bacillus cell, for example, on a temperature-sensitive plasmid, such as pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, e.g., Perego, 1993). Thus, a person of skill in the art (e.g., by reference to the rghR1, rghR2, abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, spo0F, ywjG, ywq12, BLi04199, BLi04200, licT, bg1H and bg1P gene (nucleic acid) sequences and the encoded protein sequences thereof), may readily identify nucleotide regions in the gene's coding sequence and/or the gene's non-coding sequence suitable for complete or partial deletion.

In other embodiments, a modified Bacillus cell of the disclosure is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art (e.g., see, Botstein and Shortle, 1985; Lo et al., 1985; Higuchi et al., 1988; Shimada, 1996; Ho et al., 1989; Horton et al., 1989 and Sarkar and Sommer, 1990). Thus, in certain embodiments, a gene of the disclosure is inactivated by complete or partial deletion.

In another embodiment, a modified Bacillus cell is constructed by the process of gene conversion (e.g., see Iglesias and Trautner, 1983). For example, in the gene conversion method, a nucleic acid sequence corresponding to the gene(s) is mutagenized in vitro to produce a defective nucleic acid sequence, which is then transformed into the parental Bacillus cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene (Perego, 1993). Alternatively, the defective nucleic acid sequence may contain an insertion, substitution, or deletion of one or more nucleotides of the gene, as described below.

In other embodiments, a modified Bacillus cell is constructed by established anti-sense techniques using a nucleotide sequence complementary to the nucleic acid sequence of the gene (Parish and Stoker, 1997). More specifically, expression of the gene by a Bacillus cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified Bacillus cell is produced/constructed via CRISPR-Cas9 editing. For example, a gene encoding rghR1, rghR2, abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, spo0F, ywjG, ywq12, BLi04199, BLi04200, licT, bg1H and/or bg1P can be disrupted (or deleted or down-regulated) by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Bacillus cell and a terminator active in Bacillus cell, thereby creating a Bacillus Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) protospacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Bacillus expression cassette for the gRNA is created by operably linking the DNA encoding the gRNA to a promoter active in Bacillus cells and a terminator active in Bacillus cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the Bacillus host's machinery to repair the DNA break generated by the RGEN.

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies (e.g., see Examples 6 and 7 below).

In yet other embodiments, a modified Bacillus cell is constructed by random or specific mutagenesis using methods well known in the art, including, but not limited to, chemical mutagenesis (see, e.g., Hopwood, 1970) and transposition (see, e.g., Youngman et al., 1983). Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

In certain other embodiments, a modified Bacillus cell comprises a deletion of an endogenous gene selected from rghR1, rghR2, abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, spo0F, ywjG, ywq12, BLi04199, BLi04200, licT, bg1H and bg1P. Thus, in certain of these embodiments, the modified Bacillus cell is constructed as described above.

In other embodiments, a modified Bacillus cell comprises a disruption of an endogenous gene selected from rghR1, rghR2, abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, spo0F, ywjG, ywq12, BLi04199, BLi04200, licT, bg1H and bg1P. In certain embodiments, a polynucleotide disruption cassette of the disclosure comprises a marker gene.

In other embodiments, a modified Bacillus cell comprises a down-regulated endogenous gene selected from rghR1, rghR2, abrB1, rpmJ, rpIM, BLi00412, rapK, phrK, BLi00753, yfjT, BLi00828, yhdX, yhzC, terf2, zosA, abbA, speG, yppF, BLi02543, mntR, BLi02768, sspA, BLi03127, BLi03635, mrgA, BLi03644, yvzC, spo0F, ywjG, ywq12, BLi04199, BLi04200, licT, bg1H and bg1P. For example, in certain embodiments, down-regulating one or more genes set forth above comprises deleting or disrupting the gene's upstream or downstream regulatory elements.

International PCT Publication No. WO2003/083125 discloses methods for modifying Bacillus cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E. coli and Bacillus spp.) (e.g., Ferrari et al., 1989; Saunders et al., 1984; Hoch et al., 1967; Mann et al., 1986; Holubova, 1985; Chang et al., 1979; Vorobjeva et al., 1980; Smith et al., 1986; Fisher et. al., 1981 and McDonald, 1984). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art (e.g., Stahl et al., 1984 and Palmeros et al., 2000). In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Bacillus cells are generally known on one of skill in the art. Promoter sequences of the disclosure of the disclosure are generally chosen so that they are functional in the Bacillus cells (e.g., B. licheniformis cells, B. subtilis cells and the like). Certain exemplary Bacillus promoter sequences are presented in TABLE 6. Likewise, promoters useful for driving gene expression in Bacillus cells include, but are not limited to, the B. subtilis alkaline protease (aprE) promoter (Stahl et al., 1984), the α-amylase promoter of B. subtilis (Yang et al., 1983), the α-amylase promoter of B. amyloliquefaciens (Tarkinen et al., 1983), the neutral protease (nprE) promoter from B. subtilis (Yang et al., 1984), a mutant aprE promoter (PCT Publication No. WO2001/51643) or any other promoter from B licheniformis or other related Bacilli. In certain other embodiments, the promoter is a ribosomal protein promoter or a ribosomal RNA promoter (e.g., the rrnI promoter) disclosed in U.S. Patent Publication No. 2014/0329309. Methods for screening and creating promoter libraries with a range of activities (promoter strength) in Bacillus cells is describe in PCT Publication No. WO2003/089604.

### V. Culturing Modified Cells for Production of a Protein of Interest

In other embodiments, the present disclosure provides methods for increasing the protein productivity of a modified Bacillus cell, as compared (i.e., relative) to an unmodified (parental) cell. In certain embodiments, the instant disclosure is directed to methods of producing a protein of interest (POI) comprising fermenting/cultivating a modified bacterial cell, wherein the modified cell secrets the POI into the culture medium. Fermentation methods well known in the art can be applied to ferment the modified and unmodified Bacillus cells of the disclosure.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Thus, in certain embodiments, a POI produced by a transformed (modified) host cell may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

### VI. Proteins of Interest Produced by Modified (Host) Cells

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

For example, as set forth in the Examples below, the modified (rghR2ᵣₑₛₜ) Bacillus cells of the disclosure produce increased amounts of heterologous POIs (e.g., heterologous amylases set forth in Examples 2 and 5), while showing a decrease in biomass when cultured. Thus, in certain embodiments, a modified cell of the disclosure expresses an endogenous POI, a heterologous POI or a combination of one or more thereof. For example, in certain embodiments, a modified Bacillus cell of the disclosure produces at least about 0.1% more, at least about 0.5% more, at least about 1% more, at least about 5% more, at least about 6% more, at least about 7% more, at least about 8% more, at least about 9% more, or at least about 10% or more of a POI, relative to its unmodified (parental) cell.

In certain embodiments, a modified Bacillus cell of the disclosure exhibits an increased specific productivity (Qp) of a POI relative the (unmodified) parental Bacillus cell. For example, the detection of specific productivity (Qp) is a suitable method for evaluating protein production. The specific productivity (Qp) can be determined using the following equation:

$$“ Qp = gP / gDCW \cdot hr ”$$

wherein, “gP” is grams of protein produced in the tank; “gDCW” is grams of dry cell weight (DCW) in the tank and “hr” is fermentation time in hours from the time of inoculation, which includes the time of production as well as growth time.

Thus, in certain other embodiments, a modified Bacillus cell of the disclosure comprises a specific productivity (Qp) increase of at least about 0.1%, at least about 1%, at least about 5%, at least about 6%, at least about 7%, at least about 8%, at least about 9%, or at least about 10% or more, relative to the unmodified (parental) cell.

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

For example, in certain embodiments a POI is an oxidoreductase enzyme, including, but not limited to, an EC 1 (oxidoreductase) enzyme selected from EC 1.10.3.2 (e.g., a laccase), EC 1.10.3.3 (e.g., L-ascorbate oxidase), EC 1.1.1.1 (e.g., alcohol dehydrogenase), EC 1.11.1.10 (e.g., chloride peroxidase), EC 1.11.1.17 (e.g., peroxidase), EC 1.1.1.27 (e.g., L-lactate dehydrogenase), EC 1.1.1.47 (e.g., glucose 1-dehydrogenase), EC 1.1.3.X (e.g., glucose oxidase), EC 1.1.3.10 (e.g., pyranose oxidase), EC 1.13.11.X (e.g., dioxygenase), EC 1.13.11.12 (e.g., lineolate 13S-lipozygenase), EC 1.1.3.13 (e.g., alcohol oxidase), EC 1.14.14.1 (e.g., monooxygenase), EC 1.14.18.1 (e.g., monophenol monooxigenase) EC 1.15.1.1 (e.g., superoxide dismutase), EC 1.1.5.9 (formerly EC 1.1.99.10, e.g., glucose dehydrogenase), EC 1.1.99.18 (e.g., cellobiose dehydrogenase), EC 1.1.99.29 (e.g., pyranose dehydrogenase), EC 1.2.1.X (e.g., fatty acid reductase), EC 1.2.1.10 (e.g., acetaldehyde dehydrogenase), EC 1.5.3.X (e.g., fructosyl amine reductase), EC 1.8.1.X (e.g., disulfide reductase) and EC 1.8.3.2 (e.g., thiol oxidase).

In certain embodiments a POI is a transferase enzyme, including, but not limited to, an EC 2 (transferase) enzyme selected from EC 2.3.2.13 (e.g., transglutaminase), EC 2.4.1.X (e.g., hexosyltransferase), EC 2.4.1.40 (e.g., alternasucrase), EC 2.4.1.18 (e.g., 1,4 alpha-glucan branching enzyme), EC 2.4.1.19 (e.g., cyclomaltodextrin glucanotransferase), EC 2.4.1.2 (e.g., dextrin dextranase), EC 2.4.1.20 (e.g., cellobiose phosphorylase), EC 2.4.1.25 (e.g., 4-alpha-glucanotransferase), EC 2.4.1.333 (e.g., 1,2-beta-oligoglucan phosphor transferase), EC 2.4.1.4 (e.g., amylosucrase), EC 2.4.1.5 (e.g., dextransucrase), EC 2.4.1.69 (e.g., galactoside 2-alpha-L-fucosyl transferase), EC 2.4.1.9 (e.g., inulosucrase), EC 2.7.1.17 (e.g., xylulokinase), EC 2.7.7.89 (formerly EC 3.1.4.15, e.g., [glutamine synthetase]-adenylyl-L-tyrosine phosphorylase), EC 2.7.9.4 (e.g., alpha glucan kinase) and EC 2.7.9.5 (e.g., phosphoglucan kinase).

In other embodiments a POI is a hydrolase enzyme, including, but not limited to, an EC 3 (hydrolase) enzyme selected from EC 3.1.X.X (e.g., an esterase), EC 3.1.1.1 (e.g., pectinase), EC 3.1.1.14 (e.g., chlorophyllase), EC 3.1.1.20 (e.g., tannase), EC 3.1.1.23 (e.g., glycerol-ester acylhydrolase), EC 3.1.1.26 (e.g., galactolipase), EC 3.1.1.32 (e.g., phospholipase A1), EC 3.1.1.4 (e.g., phospholipase A2), EC 3.1.1.6 (e.g., acetylesterase), EC 3.1.1.72 (e.g., acetylxylan esterase), EC 3.1.1.73 (e.g., feruloyl esterase), EC 3.1.1.74 (e.g., cutinase), EC 3.1.1.86 (e.g., rhamnogalacturonan acetylesterase), EC 3.1.1.87 (e.g., fumosin B1 esterase), EC 3.1.26.5 (e.g., ribonuclease P), EC 3.1.3.X (e.g., phosphoric monoester hydrolase), EC 3.1.30.1 (e.g., Aspergillus nuclease Si), EC 3.1.30.2 (e.g., Serratia marcescens nuclease), EC 3.1.3.1 (e.g., alkaline phosphatase), EC 3.1.3.2 (e.g., acid phosphatase), EC 3.1.3.8 (e.g., 3-phytase), EC 3.1.4.1 (e.g., phosphodiesterase I), EC 3.1.4.11 (e.g., phosphoinositide phospholipase C), EC 3.1.4.3 (e.g., phospholipase C), EC 3.1.4.4 (e.g., phospholipase D), EC 3.1.6.1 (e.g., arylsufatase), EC 3.1.8.2 (e.g., diisopropyl-fluorophosphatase), EC 3.2.1.10 (e.g., oligo-1,6-glucosidase), EC 3.2.1.101 (e.g., mannan endo-1,6-alpha-mannosidase), EC 3.2.1.11 (e.g., alpha-1,6-glucan-6-glucanohydrolase), EC 3.2.1.131 (e.g., xylan alpha-1,2-glucuronosidase), EC 3.2.1.132 (e.g., chitosan N-acetylglucosaminohydrolase), EC 3.2.1.139 (e.g., alpha-glucuronidase), EC 3.2.1.14 (e.g., chitinase), EC 3.2.1.151 (e.g., xyloglucan-specific endo-beta-1,4-glucanase), EC 3.2.1.155 (e.g., xyloglucan-specific exo-beta-1,4-glucanase), EC 3.2.1.164 (e.g., galactan endo-1,6-beta-galactosidase), EC 3.2.1.17 (e.g., lysozyme), EC 3.2.1.171 (e.g., rhamnogalacturonan hydrolase), EC 3.2.1.174 (e.g., rhamnogalacturonan rhamnohydrolase), EC 3.2.1.2 (e.g., beta-amylase), EC 3.2.1.20 (e.g., alpha-glucosidase), EC 3.2.1.22 (e.g., alpha-galactosidase), EC 3.2.1.25 (e.g., beta-mannosidase), EC 3.2.1.26 (e.g., beta-fructofuranosidase), EC 3.2.1.37 (e.g., xylan 1,4-beta-xylosidase), EC 3.2.1.39 (e.g., glucan endo-1,3-beta-D-glucosidase), EC 3.2.1.40 (e.g., alpha-L-rhamnosidase), EC 3.2.1.51 (e.g., alpha-L-fucosidase), EC 3.2.1.52 (e.g., beta-N-Acetylhexosaminidase), EC 3.2.1.55 (e.g., alpha-N-arabinofuranosidase), EC 3.2.1.58 (e.g., glucan 1,3-beta-glucosidase), EC 3.2.1.59 (e.g., glucan endo-1,3-alpha-glucosidase), EC 3.2.1.67 (e.g., galacturan 1,4-alpha-galacturonidase), EC 3.2.1.68 (e.g., isoamylase), EC 3.2.1.7 (e.g., 1-beta-D-fructan fructanohydrolase), EC 3.2.1.74 (e.g., glucan 1,4-β-glucosidase), EC 3.2.1.75 (e.g., glucan endo-1,6-beta-glucosidase), EC 3.2.1.77 (e.g., mannan 1,2-(1,3)-alpha-mannosidase), EC 3.2.1.80 (e.g., fructan beta-fructosidase), EC 3.2.1.82 (e.g., exo-poly-alpha-galacturonosidase), EC 3.2.1.83 (e.g., kappa-carrageenase), EC 3.2.1.89 (e.g., arabinogalactan endo-1,4-beta-galactosidase), EC 3.2.1.91 (e.g., cellulose 1,4-beta-cellobiosidase), EC 3.2.1.96 (e.g., mannosyl-glycoprotein endo-beta-N-acetylglucosaminidase), EC 3.2.1.99 (e.g., arabinan endo-1,5-alpha-L-arabinanase), EC 3.4.X.X (e.g., peptidase), EC 3.4.11.X (e.g., aminopeptidase), EC 3.4.11.1 (e.g., leucyl aminopeptidase), EC 3.4.11.18 (e.g., methionyl aminopeptidase), EC 3.4.13.9 (e.g., Xaa-Pro dipeptidase), EC 3.4.14.5 (e.g., dipeptidyl-peptidase IV), EC 3.4.16.X (e.g., serine-type carboxypeptidase), EC 3.4.16.5 (e.g., carboxypeptidase C), EC 3.4.19.3 (e.g., pyroglutamyl-peptidase I), EC 3.4.21.X (e.g., serine endopeptidase), EC 3.4.21.1 (e.g., chymotrypsin), EC 3.4.21.19 (e.g., glutamyl endopeptidase), EC 3.4.21.26 (e.g., prolyl oligopeptidase), EC 3.4.21.4 (e.g., trypsin), EC 3.4.21.5 (e.g., thrombin), EC 3.4.21.63 (e.g., oryzin), EC 3.4.21.65 (e.g., thermomycolin), EC 3.4.21.80 (e.g., streptogrisin A), EC 3.4.22.X (e.g., cysteine endopeptidase), EC 3.4.22.14 (e.g., actinidain), EC 3.4.22.2 (e.g., papain), EC 3.4.22.3 (e.g., ficain), EC 3.4.22.32 (e.g., stem bromelain), EC 3.4.22.33 (e.g., fruit bromelain), EC 3.4.22.6 (e.g., chymopapain), EC 3.4.23.1 (e.g., pepsin A), EC 3.4.23.2 (e.g., pepsin B), EC 3.4.23.22 (e.g., endothiapepsin), EC 3.4.23.23 (e.g., mucorpepsin), EC 3.4.23.3 (e.g., gastricsin), EC 3.4.24.X (e.g., metalloendopeptidase), EC 3.4.24.39 (e.g., deuterolysin), EC 3.4.24.40 (e.g., serralysin), EC 3.5.1.1 (e.g., asparaginase), EC 3.5.1.11 (e.g., penicillin amidase), EC 3.5.1.14 (e.g., N-acyl-aliphatic-L-amino acid amidohydrolase), EC 3.5.1.2 (e.g., L-glutamine amidohydrolase), EC 3.5.1.28 (e.g., N-acetylmuramoyl-L-alanine amidase), EC 3.5.1.4 (e.g., amidase), EC 3.5.1.44 (e.g., protein-L-glutamine amidohydrolase), EC 3.5.1.5 (e.g., urease), EC 3.5.1.52 (e.g., peptide-N(4)-(N-acetyl-beta-glucosaminyl)asparagine amidase), EC 3.5.1.81 (e.g., N-Acyl-D-amino-acid deacylase), EC 3.5.4.6 (e.g., AMP deaminase) and EC 3.5.5.1 (e.g., nitrilase).

In other embodiments a POI is a lyase enzyme, including, but not limited to, an EC 4 (lyase) enzyme selected from EC 4.1.2.10 (e.g., mandelonitrile lyase), EC 4.1.3.3 (e.g., N-acetylneuraminate lyase), EC 4.2.1.1 (e.g., carbonate dehydratase), EC 4.2.2.—(e.g., rhamnogalacturonan lyase), EC 4.2.2.10 (e.g., pectin lyase), EC 4.2.2.22 (e.g., pectate trisaccharide-lyase), EC 4.2.2.23 (e.g., rhamnogalacturonan endolyase) and EC 4.2.2.3 (e.g., mannuronate-specific alginate lyase).

In certain other embodiments a POI is an isomerase enzyme, including, but not limited to, an EC 5 (isomerase) enzyme selected from EC 5.1.3.3 (e.g., aldose 1-epimerase), EC 5.1.3.30 (e.g., D-psicose 3-epimerase), EC 5.4.99.11 (e.g., isomaltulose synthase) and EC 5.4.99.15 (e.g., (1→4)-α-D-glucan 1-α-D-glucosylmutase).

In yet other embodiments, a POI is a ligase enzyme, including, but not limited to, an EC 6 (ligase) enzyme selected from EC 6.2.1.12 (e.g., 4-coumarate:coenzyme A ligase) and EC 6.3.2.28 (e.g., L-amino-acid alpha-ligase)9

Thus, in certain embodiments, industrial protease producing Bacillus host cells provide particularly preferred expression hosts. Likewise, in certain other embodiments, industrial amylase producing Bacillus host cells provide particularly preferred expression hosts.

For example, there are two general types of proteases which are typically secreted by Bacillus spp., namely neutral (or “metalloproteases”) and alkaline (or “serine”) proteases. For example, Bacillus subtilisin proteins (enzymes) are exemplary serine proteases for use in the present disclosure. A wide variety of Bacillus subtilisins have been identified and sequenced, for example, subtilisin 168, subtilisin BPN′, subtilisin Carlsberg, subtilisin DY, subtilisin 147 and subtilisin 309 (e.g., WO 1989/06279 and Stahl et al., 1984). In some embodiments of the present disclosure, the modified Bacillus cells produce mutant (i.e., variant) proteases. Numerous references provide examples of variant proteases, such as PCT Publication Nos. WO1999/20770; WO1999/20726; WO1999/20769; WO1989/06279; U.S. RE34,606; U.S. Pat. Nos. 4,914,031; 4,980,288; 5,208,158; 5,310,675; 5,336,611; 5,399,283; 5,441,882; 5,482,849; 5,631,217; 5,665,587; 5,700,676; 5,741,694; 5,858,757; 5,880,080; 6,197,567 and 6,218,165. Thus, in certain embodiments, a modified Bacillus cells of the disclosure comprises an expression construct encoding a protease.

In certain other embodiments, a modified Bacillus cells of the disclosure comprises an expression construct encoding an amylase. A wide variety of amylase enzymes and variants thereof are known to one skilled in the art. For example, International PCT Publication NO. WO2006/037484 and WO 2006/037483 describe variant α-amylases having improved solvent stability, Publication No. WO1994/18314 discloses oxidatively stable α-amylase variants, Publication No. WO1999/19467, WO2000/29560 and WO2000/60059 disclose Termamyl-like α-amylase variants, Publication No. WO2008/112459 discloses α-amylase variants derived from Bacillus sp. number 707, Publication No. WO1999/43794 discloses maltogenic α-amylase variants, Publication No. WO1990/11352 discloses hyper-thermostable α-amylase variants, Publication No. WO2006/089107 discloses α-amylase variants having granular starch hydrolyzing activity.

In other embodiments, a POI or variant POI expressed and produced in a modified cell of the disclosure is a peptide, a peptide hormone, a growth factor, a clotting factor, a chemokine, a cytokine, a lymphokine, an antibody, a receptor, an adhesion molecule, a microbial antigen (e.g., HBV surface antigen, HPV E7, etc.), variants thereof, fragments thereof and the like. Other types of proteins (or variants thereof) of interest may be those that are capable of providing nutritional value to a food or to a crop. Non-limiting examples include plant proteins that can inhibit the formation of anti-nutritive factors and plant proteins that have a more desirable amino acid composition (e.g., a higher lysine content than a non-transgenic plant).

There are various assays known to those of ordinary skill in the art for detecting and measuring activity of intracellularly and extracellularly expressed proteins. In particular, for proteases, there are assays based on the release of acid-soluble peptides from casein or hemoglobin measured as absorbance at 280 nm or colorimetrically, using the Folin method (e.g., Bergmeyer et al., 1984). Other assays involve the solubilization of chromogenic substrates (See e.g., Ward, 1983). Other exemplary assays include succinyl-Ala-Ala-Pro-Phe-para-nitroanilide assay (SAAPFpNA) and the 2,4,6-trinitrobenzene sulfonate sodium salt assay (TNBS assay). Numerous additional references known to those in the art provide suitable methods (See e.g., Wells et al., 1983; Christianson et al., 1994 and Hsia et al., 1999).

International PCT Publication No. WO2014/164777 discloses Ceralpha α-amylase activity assays useful for amylase activities described herein.

Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS).

### EXAMPLES

Certain aspects of the present invention may be further understood in light of the following examples, which should not be construed as limiting. Modifications to materials and methods will be apparent to those skilled in the art.

### Example 1

### Duplication of 18-Bp Sequence in the rghR2 Gene of Bacillus Licheniformis Strains

Bacillus licheniformis strain DSM13 (ATTC 14580) contains a gene, designated rghR2 (BLi03647), encoding a putative HTH-type transcriptional regulator. The rghR2 nucleic acid sequence of B. licheniformis DSM13 is depicted in SEQ ID NO: 1 and the encoded amino acid sequence of the RghR2 protein of Bacillus licheniformis DSM13 is depicted in SEQ ID NO: 2.

Sequencing of the genomes of (a) B. licheniformis strain Bra7, (b) a B. licheniformis Bra7 derivative, (c) B. licheniformis strain ATCC-9789 (www.atcc.org/Products/All/9789.aspx) and (d) B. licheniformis strain ATCC-6598 (www.atcc.org/en/Products/All/6598.aspx) revealed that all of these B. licheniformis strains have a duplication of 18 nucleotides in the rghR2 gene, wherein the 18 nucleotide duplication is presented in SEQ ID NO: 3: GCCGCAGCCATTTCCAGA.

Thus, the nucleotide sequence of (a) the B. licheniformis Bra7 strain rghR2 gene (SEQ ID NO: 3) is identical to the nucleotide sequence of the rghR2 gene of (b) B. licheniformis Bra7 derivative, (c) ATCC-9789, and (d) ATCC-6598, as presented in SEQ ID NO: 3. Likewise, the amino acid sequence of the RghR2 protein of B. licheniformis Bra7 strain (SEQ ID NO: 5) is identical to the amino acid sequence of RghR2 of Bacillus licheniformis Bra7 derivative, ATCC-9789 and ATCC-6598, as presented in SEQ ID NO: 4.

An alignment of the B. licheniformis Bra7 strain RghR2 amino acid sequence (SEQ ID NO: 4) and the B. licheniformis DSM13 strain RghR2 amino acid sequence (SEQ ID NO: 2), illustrating the repeat amino acids (AAAISR) is shown in FIG. 1. The insertion of the sequence “AAAISR” is in the helix-turn-helix (HTH) domain and near the sequence-specific DNA-binding site as shown in FIG. 8. One may expect that the insertion has a significant effect on, or even completely abolishes, the “function” of the RghR2 transcription regulator (i.e., a substantially inactive transcriptional regulatory protein). For example, as a transcription regulator, RghR2 will directly and indirectly regulate the expression of several other genes. It is contemplated herein that the inactivation of RghR2 (e.g., by the 18-bp duplication set forth in SEQ ID NO: 5 encoding the “AAAISR” amino acid repeat set forth in SEQ ID NO: 6) affects cell physiology and consequently, may impact factors like growth and heterologous protein production thereof. Thus, the impact on growth and heterologous protein production were further studied by removing the 18-bp duplication in the rghR2 gene in B. licheniformis Bra7 derivative cells producing various heterologous enzymes.

### Example 2

### Removal of the 18-BP Duplication in rghR2 and its Effect on Cell Growth and Heterologous Enzyme Production

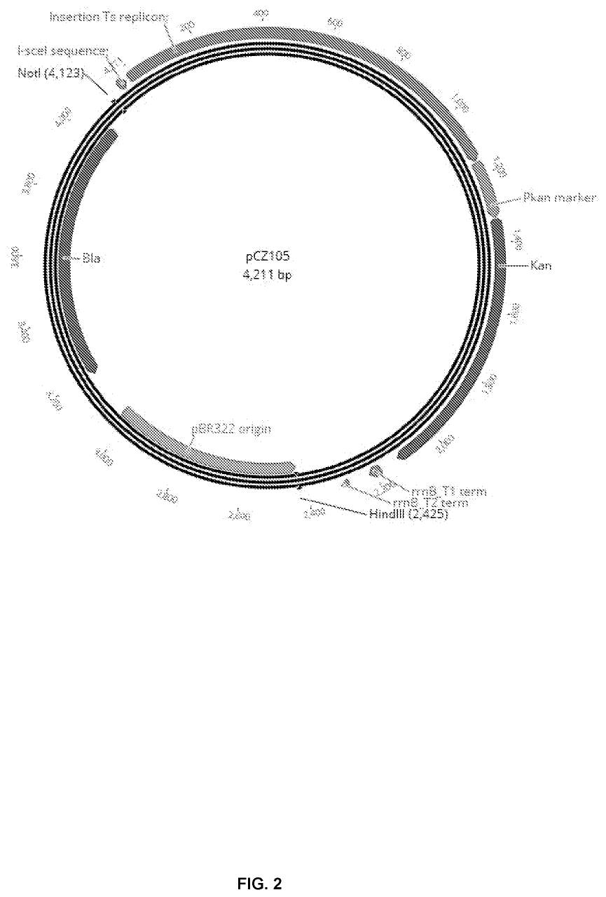

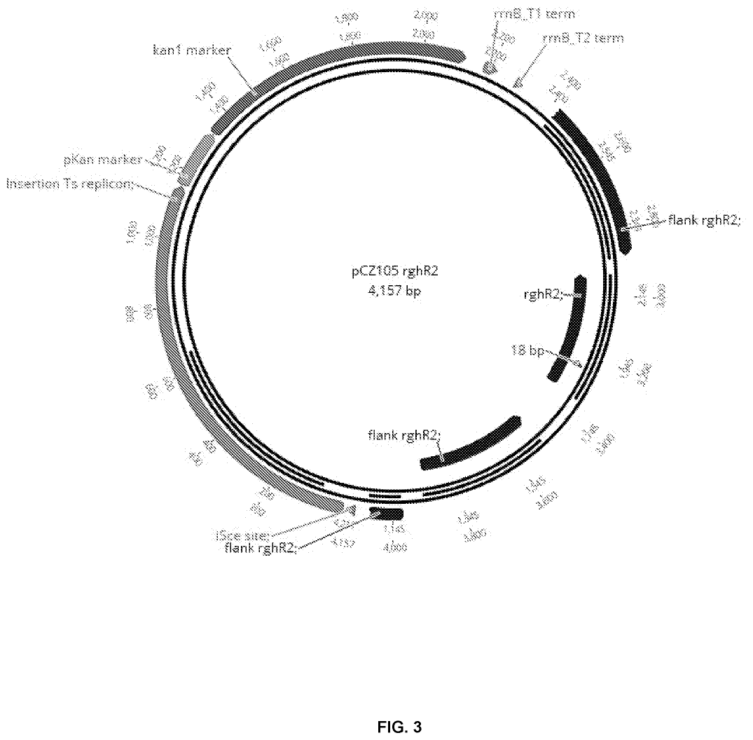

To remove the 18-bp duplication in the rghR2 gene in B. licheniformis Bra7 derivative strain (and strains related or derived from this host strain), two PCR amplifications were performed on the genomic DNA of Bra7. One PCR amplification was performed using primers 378 and 379 (TABLE 2) and a second PCR amplification was performed on genomic DNA of Bra7 using primers 380 and 381 (TABLE 2). Both fragments were gel purified and used in a fusion PCR using primer 378 and 381 to yield a rghR2 fragment with the 18-bp duplication deleted. This fragment was digested using HindIII and NotI and after gel purification ligated into a HindIII and NotI digested and gel purified temperature sensitive integration plasmid pCZ105 (FIG. 2) yielding the plasmid “pZC105_rghR2” (FIG. 3).

TABLE 2
PCR Primers

| | | SEQ |
|---|---|---|
| Primer | | ID |
| # | Nucleotide Sequence | NO |
| 369 | GAGACTAGTGAGCTCGCATCACACGC | 7 |
| 378 | GACTGCGGCCGCACCATGATTACTCCCCTTTCTAATCT | 8 |
| 379 | TCTGGAAATGGCTGCGGCGCTCACACCGGCATACATGG | 9 |
| 380 | GCCGCAGCCATTTCCAGAATCGAAAACGGCCACCGCGG | 10 |
| 381 | GACTAAGCTTCGCCGTCTTGATGCTTGT | 11 |
| 384 | GTACGGCATTTTCAGAGCCTC | 12 |
| 752 | TGAATCATCTTTCCGATCACAAGTTG | 13 |
| 753 | AAGGAGGGGATGACAAATGGAAG | 14 |

Plasmid pZC105_rghR2 was rolling circle amplified (GE Healthcare Europe GmbH, Eindhoven, The Netherlands) and transformed in a B. licheniformis (Bra7 derivative) strain that lacks the native B. licheniformis amylase (AmyL) gene, but carries an expression cassette encoding a heterologous Peanibacillus curdlanolyticus variant α-amylase. Thus, the expression cassette comprises a gene encoding a P. curdlanolyticus variant α-amylase behind a strong promoter and is integrated in the B. licheniformis genome. The sequence of the heterologous P. curdlanolyticus variant α-amylase, is disclosed in PCT Publication No. WO2014/164834 (i.e., SEQ ID NO: 35), specifically incorporated herein by reference in its entirety.

Cells were made competent using plasmid pBLComK (FIG. 4) as previously described in PCT International Application No. PCT/US2016/059078, filed Oct. 27, 2016. Cells were plated onto Luria agar containing 30 mg/l kanamycin and cultured over night at 37° C. Formed colonies were re-streaked onto fresh Luria agar and cultured over-night at 37° C. Single colonies were picked and cultured in Luria broth at 42° C. over-night while shaking to promote integration in the genome. Subsequently, cells were plated onto Luria agar containing 30 mg/l kanamycin and cultured over night at 37° C. After verification of integration in the genome by PCR using primer 369 and 384, the correct clones were cultured in Luria broth over-night at 37° C. followed by plating onto Luria agar. Single colonies were re-streaked onto LB agar plates and LB agar plates containing 30 mg/l kanamycin and cultured over night at 37° C. to verify the removal of the vector part from the genome by a double crossover event. Colonies unable to grow in the presence of kanamycin were subjected to PCR using primer 752 and 753 (TABLE 2) and the obtained fragment was sequenced. This confirmed removal of the 18-bp duplication in the rghR2 gene.

One of the verified clones, clone 197, was used for further studies. Clone 197, expressing P. curdlanolyticus α-amylase in a rghR2 restored host (i.e., rghR2ᵣₑₛₜ, removal off 18-bp duplication) and the parental control strain (i.e., comprising rghR2 with the 18-bp duplication) expressing P. curdlanolyticus α-amylase, were inoculated in tryptone soy broth (TSB) medium and cultured over night at 37° C. while shaking. Main cultures were inoculated from this pre-culture at an OD₆₆₀ of 0.1 in an amylase production medium using glucose slow release microtiter plates (srMTP; PS Biotech GmbH, Herzogenrath, Germany). Plates were cultured for 72 hours while shaking at 37° C.

After 72 hours, the OD₆₀₀ was measured (FIG. 5) and 100 ul of cells was diluted 1:1 with 50% propylene glycol (Sigma Aldrich, Zwijndrecht, The Netherlands) and incubated for 1 hour at 40° C. while shaking. After incubation, the amylase activity was measured using the Ceralpha reagent (Megazyme, Wicklow, Ireland) as described in the instructions of Megazyme, and as disclosed in PCT International Application No. PCT/US2016/059078.

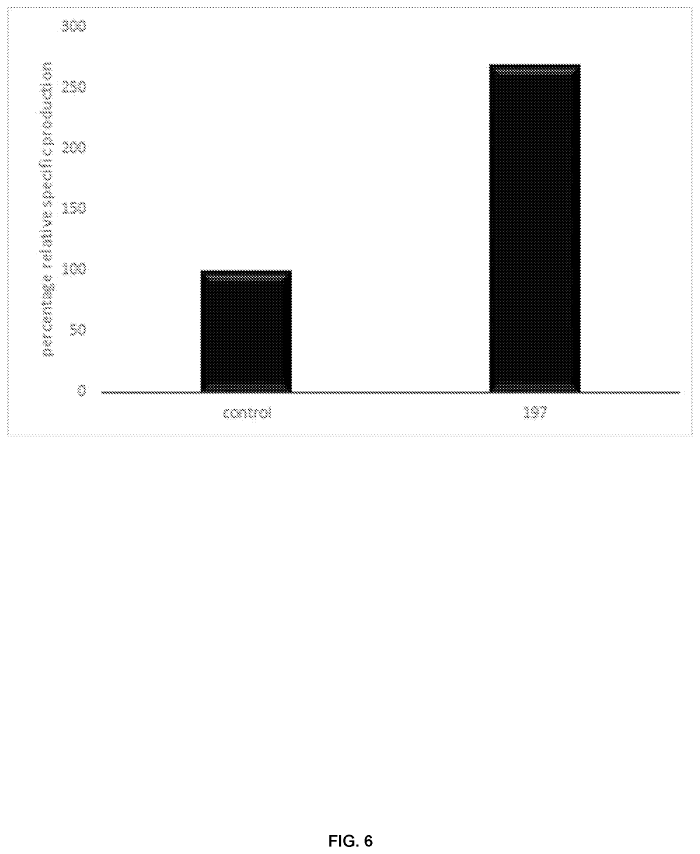

As presented in FIG. 5, deletion of the rhgR2 18-bp duplication showed a decrease in biomass when cultured, but at the same time demonstrated an improved amylase production titer. For example, FIG. 6 shows that the specific productivity (enzyme production/OD₆₀₀) of the heterologous α-amylase improved by at least a factor 2 in the rghR2 restored strain.

### Example 3

### RghR2 Regulated Genes

Transcriptome analysis of a Bra7 production strain (i.e., comprising rghR2 with the 18-bp duplication) and the rghR2 restored (rghR2ᵣₑₛₜ) variant of this strain (i.e., removal of the 18-bp duplication), revealed that transcription of several genes are regulated by RghR2 (TABLE 3). Transcription of genes downregulated by at least two-fold in the rghR2 restored strain (i.e., relative to the rghR2 inactive strain comprising the 18-bp duplication) are indicated in TABLE 4. One can expect that (further) down-regulation of these genes, or deletion of these genes, in both rghR2 restored strains and rghR2 inactivated strains (e.g., via the 18-bp insertion) has a positive effect on protein production. This would be a similar effect as seen by re-activation of rghR2 by removal of the 18-bp repeat. Therefore, the effect of inactivation of a subset of these genes (Bli03644, yvzC, abrB1, abh) on heterologous protein production was explored as described below in Example 4.

TABLE 3
GENES UPREGULATED BY A FACTOR 2 OR MORE IN A
rghR2 RESTORED STRAIN

| ID | Gene_Name | Product |
|---|---|---|
| BLi00340 | blaSE | glutamyl endopeptidase blasé (mpr) |
| BLi00343 | BLi00343 | hypothetical protein |
| BLi00373 | ycgM, putB | proline dehydrogenase YcgM |
| BLi00374 | rocA | 1-pyrroline-5-carboxylate dehydrogenase |
| BLi00401 | lchAA | lichenysin synthase LchAA |
| BLi00403 | lchAC | lichenysin synthase LchAC |
| BLi00404 | lchAD | lichenysin synthase LchAD |
| BLi01250 | catE | catechol-2,3-dioxygenase subunit CatE |
| BLi00947 | BLi00947 | hypothetical protein |
| BLi00950 | BLi00950 | hypothetical protein |
| BLi00976 | yhcM | protein YhcM |
| BLi00977 | BLi00977 | hypothetical protein |
| BLi01109 | apr | subtilisin Carlsberg |
| BLi01295 | abnA1 | arabinan endo-1,5-alpha-L-arabinosidase AbnA |
| BLi01337 | xkdK | phage tail sheath protein XkdK |
| BLi01364 | ggt | gamma-glutamyltranspeptidase |
| BLi02599 | isp | intracellular serine protease Isp |
| BLi01748 | bpr1 | bacillopeptidase F |
| BLi02215 | BLi02215 | hypothetical protein |
| BLi02255 | yvgO | stress response protein YvgO |
| BLi02264 | cwlS | D-gamma-glutamyl-meso-diaminopimelic acid |
| | | endopeptidase CwlS |
| BLi02271 | yoaJ | extracellular endoglucanase |
| BLi02544 | BLi02544 | hypothetical protein |
| BLi05030 | BLi05030 | hypothetical protein |
| BLi02827 | sacC | levanase SacC |
| BLi02828 | levG | fructose-specific phosphotransferase system |
| | | EIID component LevG |
| BLi02830 | levE | trigger enzyme fructose-specific phospho- |
| | | transferase enzyme IIB component LevE |
| BLi02831 | levD | PTS system fructose-specific transporter |
| | | subunits IIA |
| BLi05031 | BLi05031 | hypothetical protein |
| BLi03176 | ytvB | transmembrane protein YtvB |
| BLi03197 | pckA | phosphoenolpyruvate carboxykinase |
| BLi03566 | yvmC | cyclodipeptide synthase YvmC |
| BLi03567 | cypX | cytochrome P450 cyclo-l-leucyl-l-leucyl |
| | | dipeptide oxidase CypX |
| BLi03981 | BLi03981 | hypothetical protein |
| BLi03989 | pobA | 4-hydroxybenzoate 3-monooxygenase |
| BLi03991 | BLi03991 | oxidoreductase |
| BLi03992 | BLi03992 | 4-oxalocrotonate tautomerase |
| BLi03999 | yuaB | hypothetical protein |
| BLi04032 | BLi04032 | ABC transporter ATP binding/permease protein |
| BLi04124 | lanP | peptidase LanP |
| BLi04125 | lanT | lichenicidin processing transporter LanT |
| BLi04126 | lanM1 | lichenicidin modifying enzyme LanM |
| BLi05042 | lanA1 | lichenicidin prepeptide LanA |
| BLi04127 | lanA2 | lichenicidin prepeptide LanA |
| BLi04128 | lanM2 | lichenicidin modifying enzyme LanM |

Gene IDs from KEGG GENOME T00200 (Bacillus licheniformis DSM 13=ATCC 14580)

TABLE 4
GENES DOWNREGULATED BY A FACTOR 2 OR MORE
IN A rghR2 RESTORED STRAIN

| ID | Gene_Name | Product |
|---|---|---|
| BLi00050 | abrB1 | transition state transcriptional regulator AbrB |
| BLi00158 | rpmJ | 50S ribosomal protein L36 |
| BLi00167 | rplM | 50S ribosomal protein L13 |
| Bli00412 | BLi00412 | ABC transporter ATP-binding protein |
| BLi00751 | rapK | response regulator aspartate phosphatase RapK |
| BLi05046 | phrK | response regulator aspartate phosphatase RapK |
| | | regulator PhrK |
| BLi00753 | BLi00753 | SAM methytransferase |
| BLi00826 | yfjT | protein YfjT |
| BLi00828 | BLi00828 | glycerol dehydrogenase |
| BLi01035 | yhdX | protein YhdX |
| BLi01118 | yhzC | protein YhzC |
| — | terf2 | Telomeric repeat-binding factor 2 |
| BLi01593 | zosA | zinc-transporting ATPase ZosA |
| BLi01626 | abbA | AbrB inhibitor AbbA |
| BLi02012 | speG | spermidine N(1)-acetyltransferase SpeG |
| BLi02362 | yppF | protein YppF |
| BLi02543 | BLi02543 | hypothetical protein |
| BLi02623 | mntR | manganese transport transcriptional regulator |
| BLi02768 | BLi02768 | hypothetical protein |
| BLi03099 | sspA | small acid-soluble spore protein SspA |
| BLi03127 | BLi03127 | hypothetical protein |
| BLi03635 | BLi03635 | phage protein |
| BLi00972 | metQ | methionine ABC transporter substrate-binding |
| | | protein MetQ |
| BLi03478 | BLi03478 | D-alanyl-D-alanine carboxypeptidase |
| BLi03480 | mrgA | metalloregulation DNA-binding stress protein |
| | | MrgA |
| BLi03644 | BLi03644 | transcriptional regulator |
| BLi03645 | yvzC | HTH-type transcriptional regulator YvzC |
| BLi03646 | rghR1 | HTH-type transcriptional regulator RghR |
| BLi03961 | spo0F | phosphotransferase Spo0F |
| BLi03962 | ywjG | hypothetical protein |
| BLi04055 | ywql2 | hypothetical protein |
| BLi04199 | BLi04199 | family 1 glycoside hydrolase |
| BLi04200 | BLi04200 | PTS system beta-glucoside-specific |
| | | transporter subunit IIABC |
| BLi04201 | licT | transcriptional antiterminator LicT |
| BLi04214 | bglH | phospho-beta-glucosidase BglH |
| BLi04215 | bglP | trigger enzyme beta-glucoside-specific |
| | | phosphotransferase system EIIBCA component |

Gene IDs from KEGG GENOME T00200 (Bacillus licheniformis DSM 13=ATCC 14580)

### Example 4

### Inactivation of RghR2 Regulated Genes and Thier Effect on Heterologous Protein Production

The Bli03644, abrB1, yvzC and abh genes were inactivated by insertion of antibiotic marker in a Bra7 strain producing a heterologous α-amylase (i.e., the heterologous P. curdlanolyticus α-amylase disclosed in PCT Publication No. WO2014/164834), wherein the heterologous α-amylase production was determined in the four single knock-out strains (ΔBLi03644, ΔabrB1, ΔyvzC and Δabh) and compared to the parental (control) strain as described in Example 2. For example, as presented in FIG. 7, inactivation of Bli03644, abrB1, yvzC and abh resulted in improved heterologous α-amylase production, while cell growth (OD₆₀₀) was less affected.

### Example 5

### Enhanced Production of Amylases in Modified Cells Comprising rghR2ᵣₑₛₜ

In the present example, both B. licheniformis cells comprising rghR2 gene having the 18-bp duplication (SEQ ID NO: 3) and B. licheniformis cells comprising the rghR2 gene lacking the 18-bp duplication (rghR2ᵣₑₛₜ; SEQ ID NO: 1) comprise a single copy of either: (a) a heterologous Cytophaga sp. variant #1 α-amylase expression cassette integrated in the B. licheniformis genome (SEQ ID NO: 140) or (b) a variant Geobacillus stearothermophilus α-amylase expression cassette (SEQ ID NO: 141) integrated into the B. licheniformis genome, which were inoculated from a frozen vial (1 mL, 20% glycerol) in 10 mL seed medium (15 g/L Yeast extract, 5.5 g/L Dextrose, 3 g/L Potassium phosphate, 1 g/L Magnesium sulfate). Cultures were grown at 38° C. in a vented 100 mL flask at 310 RPM until the OD₆₀₀ was approximately 2. From each culture 0.25 mL was transferred to 25 mL of production medium (30 g/L 2-(N-morpholino) ethanesulfonic acid (MES), 6.7 g/L Yeast Nitrogen Base with ammonium sulfate without amino acids, 1.7 g/L Yeast Nitrogen Base without ammonium sulfate or amino acids, 0.7 g/L Soytone, pH 6.8 with Ammonium hydroxide) in a 100 mL vented flask and two 14 mm glucose feed beads were added and the flask incubated at 38° C., 310 RPM for 84 hours with periodic replacement of evaporated water losses.

After 84 hours, a sample was taken from each flask and centrifuged. One tenth (0.1) mL of the supernatant was mixed with 0.9 mL of Bradford reagent. Color was measured as absorbance of 595 nm wavelength and compared to a standard curve to determine protein concentration. The pellet was re-suspended in propylene glycol, warmed for 30 minutes and also assayed with Bradford as above. The amylase titer was determined by the aggregate of the two measurements are presented in TABLE 5.

TABLE 5
AMYLASE TITER FROM rghR2 RESTORED STRAINS

| Heterologous | | Amylase titer | Fold difference compared to rghR2 |
|---|---|---|---|
| Amylase | rghR2 allele | (g/L ± range) | 18-bp duplication (SEQ ID NO: 3) |
| Cytophaga sp. | rghR2 18-bp dup | 2.0 ± 0.1 | 1.0 |
| α-amylase (V1) | (SEQ ID NO: 3) | | |
| Cytophaga sp. | rghR2ᵣₑₛₜ | 2.2 ± 0.2 | 1.1 |
| α-amylase (V1) | (SEQ ID NO: 1) | | |
| G. stearothermophilus | rghR2 18-bp dup | 3.9 ± 0.2 | 1.0 |
| α-amylase | (SEQ ID NO: 3) | | |
| G. stearothermophilus | rghR2ᵣₑₛₜ | 4.3 ± 0.1 | 1.1 |
| α-amylase | (SEQ ID NO: 1) | | |

Thus, as presented above in TABLE 5, both B. licheniformis strains comprising the rghR2ᵣₑₛₜ allele show improvement in amylase titer of at least 10%, indicating that removing the natively existing 18-bp duplication in rghR2 gene is beneficial for production of multiple heterologous amylase molecules.

### Example 6

### CRISPR-Cas9 Editing and Deletion of the 18-Nucleotide Ducplication in the RghR2 Gene

In the present example, a gene encoding a nucleic acid guided endonuclease (e.g., Cas9 from S. pyogenes (SEQ ID NO: 91)) or a codon optimized gene thereof (e.g., Cas9 nuclease of SEQ ID NO: 92) is operably linked to a promoter active in B. licheniformis (e.g., see, TABLE 6 below) and a terminator active in B. licheniformis (e.g., SEQ ID NO: 103), thereby creating a B. licheniformis Cas9 expression cassette (SEQ ID NO: 104).

TABLE 6
LIST OF EXAMPLARY PROMOTERS
ACTIVE IN B. LICHENIFORMIS

| Promoter Name | SEQ ID NO |
|---|---|
| aprEp | 93 |
| xylAp | 94 |
| spac | 95 |
| Hyper spank | 96 |
| Vegp | 97 |
| nprEp | 98 |
| N25 promoter | 99 |
| groE promoter | 100 |
| AraAp | 101 |
| AraA2p | 102 |

A target site unique to the 18-bp duplication allele of rghR2 (SEQ ID NO: 105), such that the rghR2 gene lacking the 18-bp duplication does not contain the target site, can be identified.

Likewise, to build a DNA construct encoding a guide RNA (gRNA) targeting the unique target site within the 18-bp duplication (SEQ ID NO: 105), the variable targeting domain (VT), comprising the target site nucleotides of SEQ ID NO: 106, SEQ ID NO: 107, SEQ ID NO: 108, or SEQ ID NO: 109 (which nucleotides are upstream (5′) of the proto-spacer adjacent motif (PAM) nucleotides “TGG”, are fused to DNA encoding the Cas9 endonuclease recognition domain for S. pyogenes Cas9 (CER, SEQ ID NO: 110).

The combination (fusion) of the DNA encoding the VT domain and the DNA encoding the CER domain generate a DNA encoding a gRNA (for example the DNA encoding the gRNA targeting the target site from the 18-bp duplication within rghR2 (SEQ ID NO:105) to generate SEQ ID NO: 111.

A B. licheniformis expression cassette for the gRNA is created by operably linking the DNA encoding the gRNA (SEQ ID NO: 111) to a promoter active in B. licheniformis (e.g., TABLE 6) and a terminator active in B licheniformis (e.g., SEQ ID NO: 103) which creates a gRNA expression cassette (spac-gRNA-t0 SEQ ID NO: 112). In order to precisely repair the DNA break generated by the Cas9 expression cassette (SEQ ID NO: 104) and the gRNA expression cassette (SEQ ID NO: 112), an editing template to be used by the DNA repair machinery of the cell must be provided. For example, the 500 bp upstream (5′) of the 18-bp duplication (SEQ ID NO: 113) is fused to the 500 bp downstream (3′) of the 18-bp duplication (SEQ ID NO: 114) to generate an editing template (SEQ ID NO: 115) that can be used by the B. licheniformis host machinery to repair the DNA break generated by the RGEN.

The Cas9 expression cassette (SEQ ID NO: 104), the gRNA expression cassette (SEQ ID NO: 112) and the editing template (SEQ ID NO: 115) are co-delivered to B. licheniformis cells using many different methods (e.g., protoplast fusion, electroporation, natural competence, or induced competence). Transformed cells are screened by PCR amplifying the rghR2 locus (SEQ ID NO:116) by amplifying the locus with a forward primer (SEQ ID NO: 117) and reverse primer (SEQ ID NO: 118). These primers amplify the wild-type locus (SEQ ID NO: 116) or the restored locus that had been edited by the RGEN (SEQ ID NO: 119). These fragments are then sequenced using a sequencing primers (SEQ ID NO: 120) to identify edited colonies.

Thus, as described in this Example, any of the genes in the rghR2 regulon can be edited in a similar manner to inactivate, enhance, down-regulated or delete the gene.

### Example 7

### CRISPR-Cas9 Editing and Gene Down-Regulation

The instant Example describes the modulation (e.g., down-regulation) of a gene of interest via CRSIPR-Cas9 editing. An exemplary method to modulate gene expression level is the use of nuclease-defective variants (e.g., Cas9 D10A/N863A or D10A/H840A) of nucleotide-guided endonucleases to enhance or antagonize transcription of target gene(s). These Cas9 variants are inactive for all nuclease domains present in the protein sequence. These Cas9 variants therefore retain the RNA-guided DNA binding activity, but are unable to cleave either strand of DNA when bound to the cognate target site.

For example, the nuclease-defective Cas9 protein can be expressed as a B. licheniformis expression cassette (constructed as described in Example 6), and when combined with a B. licheniformis gRNA expression cassette, the Cas9 protein is directed to a specific target sequence within the cell. The binding of the Cas9 (variant) protein to specific target sites can block the binding or movement of transcription machinery on the DNA of the cell, thereby decreasing the amount of a gene product produced.

Additionally, the binding activity could enhance transcription by locally melting the DNA in the region allowing the transcription machinery to bind or elongate the gene more readily which would increase the amount of gene product produced. Thus, any gene in the rghR2 regulon (or any other gene in the B. licheniformis cell) can be targeted for modulation (up- or down-regulated) of gene expression using this method.

For example, to target the yvcZ gene with a nuclease defective Cas9 protein, there are 19 unique target sites within the yvcZ ORF that can be targeted (SEQ ID NO: 121 to 139). These target sequences can be made into gRNA expression cassettes, as described in Example 6.

Co-delivery of a nuclease-defective Cas9 expression cassette (e.g., constructed as described above in Example 6) with a gRNA expression cassette for the target gene allows for gene dosage changes (modulation) by silencing or activating transcription within the gene. By delivering multiple gRNA expression cassettes simultaneously, the targeting and modulation of multiple genes at the same is possible. The gene modulation (up-regulation or down-regulation) are readily monitored in cells containing the nuclease-defective Cas9 expression cassette and the gRNA expression cassette(s), by using methods such known to the skilled artisan, such as RNAseq.

### Example 8

### Enhanced Production of a Heterologous G4 Amylase in Modified Cells Comprising rghR2ᵣₑₛₜ

In the present example, B. licheniformis cells comprising a rghR2 gene having the 18-bp duplication (SEQ ID NO: 3) and B. licheniformis cells comprising the rghR2 gene lacking the 18-bp duplication (rghR2ᵣₑₛₜ; SEQ ID NO: 1), both comprise a single copy of an expression cassette (SEQ ID NO: 142) encoding a heterologous G4 amylase (variant) of Pseudomonas sp. AM1 (e.g., see PCT Publication No. WO2010/133644, specifically incorporated herein by reference in its entirety). Both strains were cultivated as described in Example 2 and samples taken after 48 hours were assayed with Ceralpha reagent as described. The fold difference in specific productivity (G4 amylase production/OD₆₀₀) in the rghR2ᵣₑₛₜ strain relative to the strain comprising the 18-bp duplication in rghR2 is presented in TABLE 7.

TABLE 7
SPECIFIC PRODUCTIVITY OF G4 AMYLASE FROM
rghR2ᵣₑₛₜ CELLS

| | | Fold difference in |
|---|---|---|
| | | Qp compared to |
| | | rghR2 w/ |
| | rghR2 allele in | 18-bp duplication |
| Heterologous Amylase | B. licheniformis host | (SEQ ID NO: 3) |
| Pseudomonas sp. | rghR2ᵣₑₛₜ | 1.25 |
| α-amylase | (SEQ ID NO: 1) | |

Thus, as presented above in TABLE 7, the specific productivity (Qp) of the heterologous G4 amylase is significantly improved in the B. licheniformis rghR2ᵣₑₛₜ cells vis-à-vis the B. licheniformis cells comprising the 18-bp duplication in the rghR2 gene.

### Example 9

### Enhanced Production of Alkaline Amylases in Modified Cells Comprising rghR2ᵣₑₛₜ

In the present example, B. licheniformis cells comprising rghR2 gene having the 18-bp duplication (SEQ ID NO: 3) and B. licheniformis cells comprising the rghR2ᵣₑₛₜ (SEQ ID NO: 1) comprise either a single copy of: (1) an expression cassette for alkaline α-amylase variant 1 integrated in the B. licheniformis genome, (2) an expression cassette of alkaline α-amylase variant 2 integrated in the B. licheniformis genome, (3) an expression cassette for alkaline α-amylase variant 3 integrated into the B. licheniformis genome or (4) an expression cassette for alkaline α-amylase variant 4 integrated into the B. licheniformis genome. Strains were fermented in a fed-batch system and at the end of the fermentations, samples were taken and assayed for alpha-amylase activity using the Ceralpha reagent of Megazyme as described in Example 2. The fold difference in amylase production in strains without the 18-bp duplication in rghR2 compared to strains with the 18-bp duplication in rghR2 is presented in TABLE 8.

TABLE 8
ALKALINE AMYLASE PRODUCTION FROM
rghR2ᵣₑₛₜ STRAINS IN FED BATCH CULTURES

| | | Fold difference compared to |
|---|---|---|
| | | rghR2 w/18-bp duplication |
| Amylase | rghR2 allele | (SEQ ID NO: 3) |
| Variant 1 | rghR2ᵣₑₛₜ (SEQ ID NO: 1) | 1.8 |
| Variant 2 | rghR2ᵣₑₛₜ (SEQ ID NO: 1) | 2.3 |
| Variant 3 | rghR2ᵣₑₛₜ (SEQ ID NO: 1) | 1.6 |
| Variant 4 | rghR2ᵣₑₛₜ (SEQ ID NO: 1) | 1.9 |

Thus, as presented above in TABLE 8, the production of alkaline amylases in fed batch cultures is improved in the B. licheniformis rghR2ᵣₑₛₜ cells vis-à-vis the B. licheniformis cells comprising the 18-bp duplication in the rghR2 gene.

### Example 10

### Enhanced Lipase Production in Bacillus Cells Comprising rghR2ᵣₑₛₜ

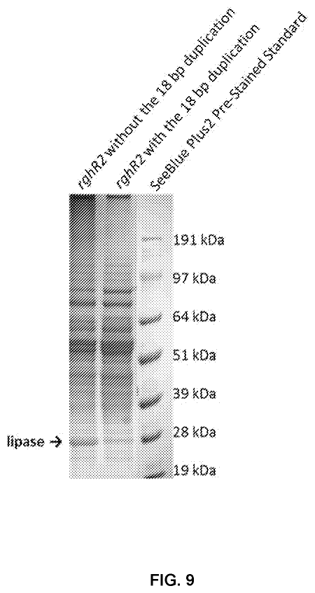

In the present example, B. licheniformis cells comprising the rghR2 gene having the 18-bp duplication (SEQ ID NO: 3) and B. licheniformis cells comprising the rghR2ᵣₑₛₜ gene (SEQ ID NO: 1) both comprise a single copy of an expression cassette encoding a heterologous EC 3.1.1.3 enzyme comprising lipase/esterase activity. Thus, both strains were cultivated as described in Example 2 and equal amounts of sample taken after 48 hours were subjected to SDS-PAGE (Invitrogen 4-12% NuPAGE Bis-Tris gel of ThermoFischer) according to the instructions of the supplier. The stained SDS-PAGE protein gel (FIG. 9) shows an increased level of the EC 3.1.1.3 enzyme (˜28 kDa) produced by the B. licheniformis rghR2ᵣₑₛₜ strain (see, FIG. 9, lane 1). Thus, as presented in FIG. 9, the production of heterologous lipase/esterase enzymes is improved in the B. licheniformis rghR2ᵣₑₛₜ cells relative to the B. licheniformis cells comprising the rghR2 gene having the 18-bp duplication.

### Example 11

### Enhanced Production of Alpha Amylase in rghR2ᵣₑₛₜ Strains in Fed Batch Culture

In the present example, B. licheniformis cells comprising rghR2 having the 18-bp duplication (SEQ ID NO: 3) and B. licheniformis cells comprising the rghR2ᵣₑₛₜ gene (SEQ ID NO: 1), both comprise a single copy of an expression cassette (SEQ ID NO: 143) encoding a heterologous Cytophaga sp. variant #2 α-amylase described in PCT Publication No. WO2014/164834. Both strains were grown under standard fed-batch fermentation conditions. Amylase activity was monitored throughout the fermentation using Ceralpha reagent of Megazyme as described in Example 2. The fold difference in the specific productivity of the B. licheniformis rghR2ᵣₑₛₜ cells relative to the B. licheniformis cells comprising the 18-bp duplication in the rghR2 gene is presented below in TABLE 9.

TABLE 9
HETEROLOGOUS AMYLASE PRODUCTION FROM
rghR2ᵣₑₛₜ STRAINS IN FED BATCH CULTURES

| | | Fold difference in |
|---|---|---|
| | | Qp compared to |
| | | rghR2 w/18-bp |
| Heterologous | | duplication |
| Amylase | rghR2 allele | (SEQ ID NO 3) |
| Cytophaga sp. | rghR2ᵣₑₛₜ (SEQ ID NO: 1) | 1.10 |
| variant #2 | | |

Thus, as presented in the TABLE 9, the specific productivity for the B. licheniformis cells producing the heterologous Cytophaga sp. variant #2 α-amylase is improved by 10% in the cells comprising the rghR2ᵣₑₛₜ gene relative to cells comprising the rghR2 gene having the 18-bp duplication.

### Example 12

### Enhanced Amylase Production in Modified B. Licheniformis Cells Comprising Alleles rghR2ᵣₑₛₜ and glcT1

In the present example, a heterologous α-amylase expression cassette was introduced into parental and modified B. licheniformis cells BF62 and BF169. More particularly, the parental B. licheniformis host, transformed with the heterologous α-amylase expression cassette, was named “BF134”. Likewise, the B. licheniformis (daughter) cell “BF62”, comprising a rghR2ᵣₑₛₜ gene, transformed with the heterologous α-amylase expression cassette, was named “BF165” and the B. licheniformis (daughter) cell “BF169”, comprising allele glcT1 and a rghr2ᵣₑₛₜ gene, was named “BF260”, as set forth below in TABLE 10.

The B. licheniformis allele glcT1 encodes a variant GlcT (transcriptional anti-termination) protein comprising a phenylalanine (F) at amino acid position 67 (F67) of the variant GlcT protein, as described in U.S. Provisional Patent Application Serial No. U.S. 62/613,339, filed Jan. 3, 2018, which is incorporated herein by reference in its entirety.

TABLE 10
B. LICHENIFORMIS PARENT/DAUGHTER
CELL MODIFICATIONS

| | | Strain Name |
|---|---|---|
| | Genetic | Transformed |
| Strain Name | Modification | w/Cassette |
| B. licheniformis (parent) | n/a | BF134 |
| BF62 (daughter) cell | rghr2ᵣₑₛₜ | BF165 |
| BF169 (daughter) cell | glcT1 + rghR2ᵣₑₛₜ | BF260 |

Thus, the parental and modified B. licheniformis BF62 and BF169 cells (TABLE 10), comprising a plasmid carrying a xylose-inducible comK expression cassette, were grown overnight at 37° C. and 250 RPM in fifteen (15) ml of L broth (1% (w/v) Tryptone, 0.5% Yeast extract (w/v), 1% NaCl (w/v)), containing one hundred (100) μg/ml spectinomycin dihydrochloride in a 125 ml baffled flask. The overnight culture was diluted to 0.7 (OD₆₀₀ units) in 25 ml fresh L broth containing one hundred (100) μg/ml spectinomycin dihydrochloride in a two hundred fifty (250) ml baffle flask. Cells were grown for one (1) hour at 37° C. (250 RPM). D-xylose was added to 0.1% (w/v) from a 50% (w/v) stock. Cells were grown for an additional four (4) hours at 37° C. (250 RPM) and pelleted at 1700×g for seven (7) minutes.

The cells were resuspended in one fourth (¼) volume of original culture using the spent medium. One hundred (100) μl of concentrated cells were mixed with approximately one (1) μg of an expression cassette comprising (in the 5′ to 3′ direction) the same 5′ catH homology arm, catH gene and spoVGrrnIp hybrid promoter, operably linked to a wild-type B. subtilis aprE 5′-UTR (WT-5′-UTR), wherein the WT-5′-UTR was operably linked to DNA encoding the lat signal sequence, followed by DNA (ORF) encoding a variant G. stearothermophilus α-amylase. The 3′ end of the DNA (ORF) encoding the variant G. stearothermophilus α-amylase, was operably linked to the lat terminator, which was operably linked to the 3′ catH homology arm. Transformation reactions were incubated at 37° C., 1000 RPM for approximately ninety (90) minutes.

Transformation mixes were plated on petri plates filled with L-broth containing ten (10) μg/ml chloramphenicol solidified with 1.5% (w/v) agar. Plates were incubated at 37° C. for two (2) days. Colonies were streak purified on petri plates filled with L-broth containing 1% (w/v) insoluble corn starch solidified with 1.5% (w/v) agar. Plates were incubated at 37° C. for twenty-four (24) hours until colonies had formed. Starch hydrolysis was indicated by clearing of the insoluble starch surrounding the colony, forming a halo, and was used to select transformants expressing the variant G. stearothermophilus α-amylase protein. Colony PCR was used to amplify the catH locus from halo producing colonies using standard techniques, and the forward and reverse primer pairs. Sequence verified B. licheniformis (daughter) cells comprising the expression cassette were stored and named as shown in the 3^{rd} column of TABLE 10.

Thus, B. licheniformis strains named BF165 (i.e., rghr2ᵣₑₛₜ) and BF260 (i.e., rghr2ᵣₑₛₜ+glcT1), comprising the α-amylase expression cassette, were assessed for α-amylase production under small scale conditions. The strains were streak purified on L agar plates containing 1% (w·v⁻¹) insoluble starch and grown for approximately twenty-four (24) hours at 37° C. A single halo positive colony was inoculated into 15 ml of Tryptic Soy Broth (1.7% (w·v⁻¹) Tryptone, 0.3% (w·v⁻¹) soytone, 0.25% (w·v⁻¹) glucose, 0.5% (w·v⁻¹) sodium chloride, 0.25% (w·v⁻¹) Dipotassium phosphate) and grown at 37° C. (250 RPM) for 6 hours. Subsequently, 0.025 ml of this seed culture was inoculated into 25 ml of flask growth medium (4% (w·v⁻¹) MES, 0.1% (w·v⁻¹) Monopotassium phosphate, 0.05% (w·v⁻¹) sodium chloride, 0.03% (w·v⁻¹) soytone, containing trace metals, pH 6.8 with Ammonium hydroxide). A single high glucose release feed bead (Kuhner) was added (feed rate 57 mg/L·hr). The cultures were grown at 42° C. (250 RPM) for 90 hours. The total secreted protein production was determined using the method of Bradford with a BSA standard. The relative α-amylase production averaged from repeat measurements of at least two independent flasks for each strain is shown in TABLE 11 below.

TABLE 11
SMALL SCALE PRODUCTION OF α-AMYLASE

| B. licheniformis cell | Modification | Relative expression ± SEM |
|---|---|---|
| BF165 | rghR2ᵣₑₛₜ | 1.00 ± 0.02 |
| BF260 | rghR2ᵣₑₛₜ + glcT1 | 1.09 ± 0.04 |

Thus, as presented in TABLE 11, the Bacillus BF260 cells (comprising a rghR2ᵣₑₛₜ and allele glcT1) demonstrate an approximately 9% increase in relative α-amylase production when compared (vis-à-vis) to Bacillus host cells BF165 (comprising rghR2ᵣₑₛₜ and a wild-type glcT gene). Thus, in certain embodiments modified B. licheniformis cells of the disclosure comprising a restored rghR2 gene (rghR2ᵣₑₛₜ), further comprises a nucleic acid construct comprising allele glcT1 (SEQ ID NO: 144), encoding a variant GlcT protein comprising a Leucine (L) to Phenylalanine (F) substitution at amino acid position 67 of the variant GlcT protein.

### REFERENCES

Albertini and Galizzi, Bacteriol., 162:1203-1211, 1985.
Bergmeyer et al., “Methods of Enzymatic Analysis” vol. 5, Peptidases, Proteinases and their Inhibitors, Verlag Chemie, Weinheim, 1984.
Botstein and Shortle, Science 229: 4719, 1985.
Brode et al., “Subtilisin BPN′ variants: increased hydrolytic activity on surface-bound substrates via decreased surface activity”, Biochemistry, 35(10):3162-3169, 1996.
Caspers et al., “Improvement of Sec-dependent secretion of a heterologous model protein in Bacillus subtilis by saturation mutagenesis of the N-domain of the AmyE signal peptide”, Appl. Microbiol. Biotechnol., 86(6):1877-1885, 2010.
Chang et al., Mol. Gen. Genet., 168:11-115, 1979.
Christianson et al., Anal. Biochem., 223:119-129, 1994.
Devereux et al., Nucl. Acid Res., 12: 387-395, 1984.
Earl et al., “Ecology and genomics of Bacillus subtilis”, Trends in Microbiology., 16(6):269-275, 2008.
Ferrari et al., “Genetics,” in Harwood et al. (ed.), Bacillus, Plenum Publishing Corp., 1989.
Fisher et. al., Arch. Microbiol., 139:213-217, 1981.
Guerot-Fleury, Gene, 167:335-337, 1995.
Hamoen et al., “Controlling competence in Bacillus subtilis: shared used of regulators”, Microbiology, 149:9-17, 2003.
Hamoen et al., Genes Dev. 12:1539-1550, 1998.
Hampton et al., Seroloaical Methods, A Laboratory Manual, APS Press, St. Paul, MN, 1990.
Hardwood and Cutting (eds.) Molecular Biological Methods for Bacillus, John Wiley & Sons, 1990.
Hayashi et al., Mol. Microbiol., 59(6): 1714-1729, 2006
Higuchi et al., Nucleic Acids Research 16: 7351, 1988.
Ho et al., Gene 77: 61, 1989.
Hoch et al., J. Bacteriol., 93:1925-1937, 1967.
Holubova, Folia Microbiol., 30:97, 1985.
Hopwood, The Isolation of Mutants in Methods in Microbiology (J. R. Norris and D. W. Ribbons, eds.) pp 363-433, Academic Press, New York, 1970.
Horton et al., Gene 77: 61, 1989.
Hsia et al., Anal Biochem., 242:221-227, 1999.
Iglesias and Trautner, Molecular General Genetics 189: 73-76, 1983.
Jensen et al., “Cell-associated degradation affects the yield of secreted engineered and heterologous proteins in the Bacillus subtilis expression system” Microbiology, 146 (Pt 10:2583-2594, 2000.
Liu and Zuber, 1998,
Lo et al., Proceedings of the National Academy of Sciences USA 81: 2285, 1985.
Maddox et al., J. Exp. Med., 158:1211, 1983.
Mann et al., Current Microbiol., 13:131-135, 1986.
McDonald, J. Gen. Microbiol., 130:203, 1984.
Needleman and Wunsch, J. Mol. Biol., 48: 443, 1970.
Ogura & Fujita, FEMS Microbiol Lett., 268(1): 73-80. 2007.
Olempska-Beer et al., “Food-processing enzymes from recombinant microorganisms—a review”’ Regul. Toxicol. Pharmacol., 45(2):144-158, 2006.
Palmeros et al., Gene 247:255-264, 2000.
Parish and Stoker, FEMS Microbiology Letters 154: 151-157, 1997.
Pearson and Lipman, Proc. Natl. Acad. Sci. USA 85: 2444, 1988.
Perego, 1993, In A. L. Sonneshein, J. A. Hoch, and R. Losick, editors, Bacillus subtilis and Other Gram-Positive Bacteria, Chapter 42, American Society of Microbiology, Washington, D.C.
Raul et al., “Production and partial purification of alpha amylase from Bacillus subtilis (MTCC 121) using solid state fermentation”, Biochemistry Research International, 2014.
Sarkar and Sommer, BioTechniques 8: 404, 1990.
Saunders et al., J. Bacteriol., 157:718-726, 1984.
Shimada, Meth. Mol. Biol. 57: 157; 1996
Smith and Waterman, Adv. Appl. Math., 2: 482, 1981.
Smith et al., Appl. Env. Microbiol., 51:634 1986.
Stahl and Ferrari, J. Bacteriol., 158:411-418, 1984.
Stahl et al, J. Bacteriol., 158:411-418, 1984.
Tarkinen, et al, J. Biol. Chem. 258: 1007-1013, 1983.
Trieu-Cuot et al., Gene, 23:331-341, 1983.
Van Dijl and Hecker, “Bacillus subtilis: from soil bacterium to super-secreting cell factory”, Microbial Cell Factories, 12(3). 2013.
Vorobjeva et al., FEMS Microbiol. Lett., 7:261-263, 1980.
Ward, “Proteinases,” in Fogarty (ed.)., Microbial Enzymes and Biotechnology. Applied Science, London, pp 251-317, 1983.
Wells et al., Nucleic Acids Res. 11:7911-7925, 1983.
Westers et al., “Bacillus subtilis as cell factory for pharmaceutical proteins: a biotechnological approach to optimize the host organism”, Biochimica et Biophysica Acta., 1694:299-310, 2004.
Yang et al, J. Bacteriol., 160: 15-21, 1984.
Yang et al., Nucleic Acids Res. 11: 237-249, 1983.
Youngman et al., Proc. Natl. Acad. Sci. USA 80: 2305-2309, 1983.

1. A method for restoring the activity of a substantially inactive RghR2 protein in a B. licheniformis cell, the method comprising:
obtaining a B. licheniformis cell comprising a gene encoding a RghR2 protein comprising 90% sequence identity to SEQ ID NO: 4, wherein gene encoding the RghR2 protein comprising 90% sequence identity to SEQ ID NO: 4 comprises an 18-nucleotide duplication in the rghR2 gene which corresponds to a repeat of amino acids AAAISR at amino acid positions 38-43 of the RghR2 protein of SEQ ID NO: 4, and
modifying the cell of by deleting the 18-nucleotide duplication in the rghR2 gene to yield a rghR2ᵣₑₛₜ gene encoding an active RghR2 protein comprising 90% sequence identity to SEQ ID NO: 2.

2. A method for increasing the production of a heterologous protein of interest in B. licheniformis cells comprising:
obtaining a B. licheniformis cell comprising a gene encoding a RghR2 protein comprising at least 90% sequence identity to SEQ ID NO: 4, wherein gene encoding the RghR2 protein of SEQ ID NO: 4 comprises an 18-nucleotide duplication in the rghR2 gene which corresponds to a repeat of amino acids AAAISR at amino acid positions 38-43 of the RghR2 protein of SEQ ID NO: 4,
introducing into the cell an expression cassette encoding the heterologous POI, genetically modifying the cell of by deleting the 18-nucleotide duplication in the rghR2 gene to yield a rghR2ᵣₑₛₜ gene encoding a RghR2 protein comprising 90% sequence identity to SEQ ID NO: 2,
cultivating the modified cell in a medium suitable for the production of the heterologous POI, and
recovering the heterologous POI from the cultivation medium or cell lysate,
wherein the modified cell produces an increased amount of the heterologous POI, relative to a control B. licheniformis cell are cultivated under the same conditions, wherein the control cell comprises the introduced expression cassette encoding the heterologous POI and the gene encoding the RghR2 protein of SEQ ID NO: 4 comprising the 18-nucleotide duplication in the rghR2 gene.


SEQUENCE LISTING (144 sequences)

SEQ ID NO: 1 - DNA, 405 bp, Bacillus licheniformis
  source 1..405: mol_type = other DNA
atggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataa

SEQ ID NO: 2 - protein, 134 aa, Bacillus licheniformis
MAMTRFGERLKELREQRSLSVNQLAMYAGVSAAAISRIENGHRGVPKPATIRKLAEALKMPYEQLMDIAGYMRADEIREQPRGYVTMQEIAAKHGVEDLWLFKPEKWDCLSREDLLNLEQYFHFLVNEAKKRQS

SEQ ID NO: 3 - DNA, 423 bp, Bacillus licheniformis
  source 1..423: mol_type = other DNA
atggcgatgacaaggttcggcgagcggctcaaagagctgagggaacaaagaagcctgtcggttaatcagcttgccatgtatgccggtgtgagcgccgcagccatttccagagccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataa

SEQ ID NO: 4 - protein, 140 aa, Bacillus licheniformis
MAMTRFGERLKELREQRSLSVNQLAMYAGVSAAAISRAAAISRIENGHRGVPKPATIRKLAEALKMPYEQLMDIAGYMRADEIREQPRGYVTMQEIAAKHGVEDLWLFKPEKWDCLSREDLLNLEQYFHFLVNEAKKRQS

SEQ ID NO: 5 - protein, 6 aa, Bacillus licheniformis
AAAISR

SEQ ID NO: 6 - protein, 12 aa, Bacillus licheniformis
AAAISRAAAISR

SEQ ID NO: 7 - DNA, 26 bp, synthetic construct
  misc_feature 1..26: note = primer
  source 1..26: mol_type = other DNA
gagactagtgagctcgcatcacacgc

SEQ ID NO: 8 - DNA, 38 bp, synthetic construct
  misc_feature 1..38: note = primer
  source 1..38: mol_type = other DNA
gactgcggccgcaccatgattactcccctttctaatct

SEQ ID NO: 9 - DNA, 38 bp, synthetic construct
  misc_feature 1..38: note = primer
  source 1..38: mol_type = other DNA
tctggaaatggctgcggcgctcacaccggcatacatgg

SEQ ID NO: 10 - DNA, 38 bp, synthetic construct
  misc_feature 1..38: note = primer
  source 1..38: mol_type = other DNA
gccgcagccatttccagaatcgaaaacggccaccgcgg

SEQ ID NO: 11 - DNA, 28 bp, synthetic construct
  misc_feature 1..28: note = primer
  source 1..28: mol_type = other DNA
gactaagcttcgccgtcttgatgcttgt

SEQ ID NO: 12 - DNA, 21 bp, synthetic construct
  misc_feature 1..21: note = primer
  source 1..21: mol_type = other DNA
gtacggcattttcagagcctc

SEQ ID NO: 13 - DNA, 26 bp, synthetic construct
  misc_feature 1..26: note = primer
  source 1..26: mol_type = other DNA
tgaatcatctttccgatcacaagttg

SEQ ID NO: 14 - DNA, 23 bp, synthetic construct
  misc_feature 1..23: note = primer
  source 1..23: mol_type = other DNA
aaggaggggatgacaaatggaag

SEQ ID NO: 15 - DNA, 402 bp, Bacillus licheniformis
  source 1..402: mol_type = other DNA
atgacgaactttggacaccatttacgacaattaagggaacggaaaaaactgaccgtcaatcaactggcgatgtattccggcgtcagttcggcaggcatttcgcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactga

SEQ ID NO: 16 - protein, 60 aa, Bacillus licheniformis
MTNFGHHLRQLRERKKLTVNQLAMYSGVSSAGISRIENGKRGVPKPATIRKLADALKVPY

SEQ ID NO: 17 - DNA, 231 bp, Bacillus licheniformis
  source 1..231: mol_type = other DNA
atggtaaaaaagattcatatcaaaagagattttgttctccagtacatgattgaacacaatctctccttaaatcagcttgccattgaaatcggcgtatccccggcgacactcagcagagttttaaatggcgaaaggaggcccggacaacttgtgatcggaaagatgattcagtatttcaacaaaaaatttgaagatctcttttattataaagatgttgacaaaagtcaataa

SEQ ID NO: 18 - protein, 75 aa, Bacillus licheniformis
MVKKIHIKRDFVLQYMIEHNLSLNQLAIEIGVSPATLSRVLNGERRPGQLVIGKMIQYFNKKFEDLFYYKDVDKS

SEQ ID NO: 19 - DNA, 285 bp, Bacillus licheniformis
  source 1..285: mol_type = other DNA
gtgaaaaataccgggattgtccggagaatcgatgagctcggcagagtcgttctcccggtcgaaatgcgcagggtgctgaatatcaatgaaaaggacccgctcgaaatatataccgacggcgaaaacatcattttgacaaaatacgccgcaaacatggcatgtttgatgaccggcgacatcaccacgaaaaataaaacgtatgcgggcggcaaaatcgtactcagcccgcgcggagcggaaatgctcctggaagatatgatggcggcactgtcagaaaagaaataa

SEQ ID NO: 20 - protein, 93 aa, Bacillus licheniformis
MKNTGIVRRIDELGRVVLPVEMRRVLNINEKDPLEIYTDGENIILTKYAANMACLMTGDITTKNKTYAGGKIVLSPRGAEMLLEDMMAALSEK

SEQ ID NO: 21 - DNA, 285 bp, Bacillus licheniformis
  source 1..285: mol_type = other DNA
atgaaatctacaggtattgtacgtaaagttgatgaactaggacgcgtggtgattccaatcgaacttcgccgtacgcttggaatcgcagaaaaagacgctcttgaaatctatgtagatgacgaaaaaatcatcttgaaaaaatataaaccaaacatgacttgccaagttacaggtgaggtttctgatgacaaccttaaacttgcaggcggtaaattggttcttagccctgaaggcgctgagcaaatcattaacgaaattcaagcacaacttcaatctcaaaaataa

SEQ ID NO: 22 - protein, 94 aa, Bacillus licheniformis
MKSTGIVRKVDELGRVVIPIELRRTLGIAEKDALEIYVDDEKIILKKYKPNMTCQVTGEVSDDNLKLAGGKLVLSPEGAEQIINEIQAQLQSQK

SEQ ID NO: 23 - DNA, 288 bp, Bacillus licheniformis
  source 1..288: mol_type = other DNA
ttgaaagcaataggagttgtgagaaaagtagacgaactcggcaggatcgtgatgccgattgaattgagaagagctttggatatctcgatcaaggacagcatcgaattctttgtcgaccaagataaaatcgtcctgaaaaaatataaaccgcacggtgtgtgtctgatgaccggtgaaatcacttctgaaaaccgcgagtatggaaacgggaaaattacgctcagtcctgaaggggccgaattgcttttggaagaaatcaaagctgccttaaacggagtgaaagcataa

SEQ ID NO: 24 - protein, 95 aa, Bacillus licheniformis
MKAIGVVRKVDELGRIVMPIELRRALDISIKDSIEFFVDQDKIVLKKYKPHGVCLMTGEITSENREYGNGKITLSPEGAELLLEEIKAALNGVKA

SEQ ID NO: 25 - DNA, 114 bp, Bacillus licheniformis
  source 1..114: mol_type = other DNA
atgaaagtgagaccatcagttaaaccgatctgtgaaaaatgcaaggtcattcgcagaaaaggaaaagtaatggtgatctgtgaaaatccaaagcataaacaaaaacaaggataa

SEQ ID NO: 26 - protein, 37 aa, Bacillus licheniformis
MKVRPSVKPICEKCKVIRRKGKVMVICENPKHKQKQG

SEQ ID NO: 27 - DNA, 431 bp, Bacillus licheniformis
  source 1..431: mol_type = other DNA
atgcgtacaacacctatggctaacgcaagtaatattgaacgcaagtggttagttgttgatgctgctggcaagacgctaggacgtctttctactgaagttgcatctatccttcgcggaaaacataaaccaacttacacaccacacgttgacactggagatcatgtgatcatcatcaacgctgaaaaaatcgagttaactggtaaaaagttaacggacaaaatctactaccgtcacactcaacatccaggcggtttaaaatcaagaactgctcttgaaatgcgtacaaactaccctgagaaaatgcttgaacttgcgatcaaaggcatgcttccaaaaggttctctaggtcgtcaaatgttcaaaaaattgaatgtataccgtggttctgagcatccacaccaagcacaaaaacctgaagtttacgaacttcg

SEQ ID NO: 28 - protein, 143 aa, Bacillus licheniformis
MRTTPMANASNIERKWLVVDAAGKTLGRLSTEVASILRGKHKPTYTPHVDTGDHVIIINAEKIELTGKKLTDKIYYRHTQHPGGLKSRTALEMRTNYPEKMLELAIKGMLPKGSLGRQMFKKLNVYRGSEHPHQAQKPEVYEL

SEQ ID NO: 29 - DNA, 915 bp, Bacillus licheniformis
  source 1..915: mol_type = other DNA
atgggtacaatagggataaactttaaaaacattcaacatcaatatcgtaccaaggatgtgctaaggggggtgagtttccatgccgatccctcctccatcacgttcttggcaggcgagaatggagccggaaagacaactttgataaaggttgctctcggcttaatcagtccgaaggcaggaaatgcgctgttcgacgggcaaagcgtcggggaaatccgggagaagatcagctgcgtgtttgatgagcctcctgtttatcctaatgaaagtggtcttgacaatctaaagtttttatcaggtatacacagccttgatcgaaagtggagtcaggaggtttgtgccatgctcaaattggatgagggacttttaaaacaaaaagcaaaagcgctatcactgggtcaaagacaccgtttagctgtagcggccgcgttattgcgcaaacctaaatatttgtttcttgatgagccgtcaatcggcctcgacccaccgtcatggcagctggtccaaatcgctttaaaacagatgactgccaggggatgtgcaattttgattacggggcaaaattatgacgcgattgaaaatctcgctgacaatatagcgattttgcaaagtggaaaaattatcttctccggtcctattgtaaaacttgttcaagcatttcccgtatatgttcgaattgtaactgatgaccatcgggaaatagctgtgcagtttccagaagctgaacctgattcttccggaatataccgcatcgtttgcgaatcaggtgaacaggcccgcgccgtgattgatcaggtgaggagaagcaccctgaattttcaagagctatcgactgaaaaagcgtcaatggggaaaatggtgacagatatttacaaggatggatttcagtcggaaaagagagggttaacgtatgaa

SEQ ID NO: 30 - protein, 304 aa, Bacillus licheniformis
MGTIGINFKNIQHQYRTKDVLRGVSFHADPSSITFLAGENGAGKTTLIKVALGLISPKAGNALFDGQSVGEIREKISCVFDEPPVYPNESGLDNLKFLSGIHSLDRKWSQEVCAMLKLDEGLLKQKAKALSLGQRHRLAVAAALLRKPKYLFLDEPSIGLDPPSWQLVQIALKQMTARGCAILITGQNYDAIENLADNIAILQSGKIIFSGPIVKLVQAFPVYVRIVTDDHREIAVQFPEAEPDSSGIYRIVCESGEQARAVIDQVRRSTLNFQELSTEKASMGKMVTDIYKDGFQSEKRGLTY

SEQ ID NO: 31 - DNA, 1096 bp, Bacillus licheniformis
  source 1..1096: mol_type = other DNA
atgagcaaggtcgcttcagaggtggttgcgggggtgttgaacgaattccatttggccattaaaaagcacgatactgagcaggcaaagcatctttttgatgaggcgaagtcgatgttttccgagatggaagaggatcagaatgtgctggcttatttttcgcttcttgaggagcggtaccggatgatgctttatgatgcgaggggagagcggctgccgcgggagtcttattttaatgattcgcagatcgagtgcatcgagcagacggatcatatgattgattactatttctacttttttgaggcgatgcatgaagcgtacaacaagaatgttgagcgggcgatcagtctgtacaaggttgccgagaaaaaactggcgaaggtgcccgatcagattgaagcggccgagttttattttaaagtgtcctggctgtatatgtctcttcggcaaaatgcggtttctctcaattatgcgagagacgcgatgaatatttacaaaatgcatgacgggtacgaaaaaaagctggcgatttcccaagttgtgatggggacaaattacatgcagatgcagcgctttaaagatgcggagaagtactttgaagaatcgattgaaatttccaaaaagattgacgattcatttttagaagcgatgcttcatcacaatatcagcattctgtattccaattccggccggtctcaggaatgcattctcgccgtccagcatgctttgagcaacgccgaatggtgcaagtcaagctactatatcaactcgctttacatgctgaccagagagtttttcaaaatcggcgaaacagaagcggccctgttctatcataaaaaaggacaggaggaattaaagaaaaacgggaataagcattatgaaaagaaaataaatattatttatgagctgtattgccatgaaaacgtaaaaagcatcaaagacgacatccattccttggacgagatgaatgatttagacggtgtctgcgatctttctttgctcatctcaagctattttgagaaaaaaggagatgacaagaaagcgctggaatttgttaaaatattcatgaaagccgaaaacaaaatgagatcattaggga

SEQ ID NO: 32 - protein, 364 aa, Bacillus licheniformis
MSKVASEVVAGVLNEFHLAIKKHDTEQAKHLFDEAKSMFSEMEEDQNVLAYFSLLEERYRMMLYDARGERLPRESYFNDSQIECIEQTDHMIDYYFYFFEAMHEAYNKNVERAISLYKVAEKKLAKVPDQIEAAEFYFKVSWLYMSLRQNAVSLNYARDAMNIYKMHDGYEKKLAISQVVMGTNYMQMQRFKDAEKYFEESIEISKKIDDSFLEAMLHHNISILYSNSGRSQECILAVQHALSNAEWCKSSYYINSLYMLTREFFKIGETEAALFYHKKGQEELKKNGNKHYEKKINIIYELYCHENVKSIKDDIHSLDEMNDLDGVCDLSLLISSYFEKKGDDKKALEFVKIFMKAENKMRSL

SEQ ID NO: 33 - DNA, 121 bp, Bacillus licheniformis
  source 1..121: mol_type = other DNA
atgaaaaaattaattctttgcttgtcgttaactgctatggtcttaggcggagctgctttatcccaaagccacaatcaggcgtcaggcggcgttcaaacagcggagctgccggttgggggtt

SEQ ID NO: 34 - protein, 40 aa, Bacillus licheniformis
MKKLILCLSLTAMVLGGAALSQSHNQASGGVQTAELPVGG

SEQ ID NO: 35 - DNA, 747 bp, Bacillus licheniformis
  source 1..747: mol_type = other DNA
atgaatgacaaaacatttttatcatttttaaaacacgcagataagcctttcagcggctgggatttttctttcattgaagacacaggacgaatgaaaagcgacctgctttcatggtcatacggaagcatggctctgtctcttatccaggattccgaatcaatgttggatatggggacaggcggcggcgagtttttatccaaattggggccgtttccttcgtcagcatacgctactgaatgttatttgcctaatgtgccagtcgccaaggaacgattgacgcctttaggggttcaggtcgttcaaattgatgatgatgaagatcttccatttgaatccggccaattcgacctgatcatcaataaacacgaatcatattcagtacaagaggtgaggagaatcctttcaaaaggaggacggtttctcactcagcaagtcggcgggcttgattgcgaagaaataaatgaaaaacttggcgtgccgctaaatgaagaatttaaggattgggacttggaaacagcgttaaaagagatggaaaagcatgattttaagattttaaaaagcagagaagagtgtccgactcaaaggttttatgatattggggctctggtctattatttgaaagccattccctggcaggcgctcggttttgaagtgaatcaatataaggatgaattgtacgagattcataaaatgatcgaagaaaagggctattttgacgtcacacagtaccggtttatgattttagcg

SEQ ID NO: 36 - protein, 248 aa, Bacillus licheniformis
MNDKTFLSFLKHADKPFSGWDFSFIEDTGRMKSDLLSWSYGSMALSLIQDSESMLDMGTGGGEFLSKLGPFPSSAYATECYLPNVPVAKERLTPLGVQVVQIDDDEDLPFESGQFDLIINKHESYSVQEVRRILSKGGRFLTQQVGGLDCEEINEKLGVPLNEEFKDWDLETALKEMEKHDFKILKSREECPTQRFYDIGALVYYLKAIPWQALGFEVNQYKDELYEIHKMIEEKGYFDVTQYRFMIL

SEQ ID NO: 37 - DNA, 183 bp, Bacillus licheniformis
  source 1..183: mol_type = other DNA
atgggtaacgcagtacatgacaaagaacagcaagtcaattatttgaaaaacagattggatatgtttatgtcagtcatcgattctttagacccggaatcgaccgaccttgaagatattgacagactgatcagcatgctcgacgatttggaagccaaatacgagcgctttaaaaaagactggaaa

SEQ ID NO: 38 - protein, 61 aa, Bacillus licheniformis
MGNAVHDKEQQVNYLKNRLDMFMSVIDSLDPESTDLEDIDRLISMLDDLEAKYERFKKDWK

SEQ ID NO: 39 - DNA, 1086 bp, Bacillus licheniformis
  source 1..1086: mol_type = other DNA
atgtcaaaatcagtaaaatcagtcacatcacctaaaaaatttattacaggaaaacgactgctggagaacttgaacgactacattgaagattttggcgacaacgcatatatcatttgcgatgaattcattttggaacgcgctcaaaaagaagcggggaattcgattcagaaagccggcaatcaagccgtttttgaaaaattcaattacgaatgcacacaggaagaaatcgatcgcaaccgggagcttgcacgcaatgcaggcgctaatatcatcgttgggatcggaggcggtaaaacgcttgataccgcaaaagccaccgcttattacgagaagctgccggttgtgattttcccgacaattgcttctacggatgctccatgtacggcccttgccgtcatttataaacacgacggatcgtttgaccgctatctgtttttgccgacgaacccagatgtcgttcttgcggactctgagattttggcatccgcgccgccgcgctttttcgcagccggtatcggtgacgccttggcgacgtattttgaagcgcgtgcctgctttaaagcaaacggcgataacctcgtgctgatgaagccttcaacaactggattgggacttgcccgtctttgctatgatacgctgttggaaaacggtgtgaaagcgatgcaggcggttaagcacggcgtttccacacgagcggtcgaagatacaatcgaggcgaccatctatttaagcggcgtcggtgccgaatcaggcggtcttgccgccgcacacgcgatccacaacggaatgacagccgttccttctctgcacagggctcagcacggcgaaaaagtcacgttcggccttttggcgcagcttgttcttgaaaacgcgccggccgaagaattggagaccgttattgactttatcaaaggcgtcggtcttccgttgacattaaaagacctcggagtcgacgaatttgtcgaagaagaatggcgccaagtcgctcaaagcgcttgcgcggaaggcgacacaatgggcaacatgccgttcccagtcacccctgacgacgtctacaatgcgatcgtcgccgccaacgcgattgca

SEQ ID NO: 40 - protein, 361 aa, Bacillus licheniformis
MSKSVKSVTSPKKFITGKRLLENLNDYIEDFGDNAYIICDEFILERAQKEAGNSIQKAGNQAVFEKFNYECTQEEIDRNRELARNAGANIIVGIGGGKTLDTAKATAYYEKLPVVIFPTIASTDAPCTALAVIYKHDGSFDRYLFLPTNPDVVLADSEILASAPPRFFAAGIGDALATYFEARACFKANGDNLVLMKPSTTGLGLARLCYDTLLENGVKAMQAVKHGVSTRAVEDTIEATIYLSGVGAESGGLAAAHAIHNGMTAVPSLHRAQHGEKVTFGLLAQLVLENAPAEELETVIDFIKGVGLPLTLKDLGVDEFVEEEWRQVAQSACAEGDTMGNMPFPVTPDDVYNAIVAANAI

SEQ ID NO: 41 - DNA, 110 bp, Bacillus licheniformis
  source 1..110: mol_type = other DNA
atgatgggtaaagggagaattaaagtggaagaacggattaagatcgaaaccgatgctgaaatgtttaaagcgactctccttgatcaaacacagtctcagaagaagaaata

SEQ ID NO: 42 - protein, 36 aa, Bacillus licheniformis
MMGKGRIKVEERIKIETDAEMFKATLLDQTQSQKKK

SEQ ID NO: 43 - DNA, 231 bp, Bacillus licheniformis
  source 1..231: mol_type = other DNA
atgaaagagaaaaaatcgtacgctgagctcatgaagtcccgcaatacccaaaaggtgaaagaacttgatgtaaccatcacggatatctacattcaaatggtccttgatgaatcgctttttaaacggcgtttgcacacgctgagcaagaagattaatgaagcattagacaaaggagataagcaatctttccttgagctttcaagagaatatacagcgctgaaaaagcacgca

SEQ ID NO: 44 - protein, 77 aa, Bacillus licheniformis
MKEKKSYAELMKSRNTQKVKELDVTITDIYIQMVLDESLFKRRLHTLSKKINEALDKGDKQSFLELSREYTALKKHA

SEQ ID NO: 45 - DNA, 627 bp, Bacillus licheniformis
  source 1..627: mol_type = other DNA
gtgggaaaagaaaaaacaaaaaagccaatttataagaaatggtggttttggttaatcatcgttatcatcatcggggcagcagcatcaaatggtggaaattcagagcaagcttcatcaacaaataaagaaaaatccaccgagagtaaaacgactgaaacaaaacaagatacgaagaaagaagaaaaaaaggaagaaccgaaaaaagaggaaacaaaccctaaaatcggtgacgatgttaaagtcggtgatatgaattataaaatcactgggaagaaaacagcagatcaggtgggaccgtctgcattgcctcaaaaagctagcgataaataccttgttattgatgtcacattgaaaaataacggcaatgaaaaagtaacagtagacgcttctttcttcaagcttaaacgtggagaaaaaacctatgaagctgattctgctgcaagcatgtcagcgaaccaaagcgaggacggcaatattgacaataactttttccttcaaaacttgaatcctgattctaaaatcagtggaaaagtagtatttgatgtagctccggaagttgctaacgcaaaagacctacaattacaagtgcagactggtgcatggggaacggaaaccggaatcatcgat

SEQ ID NO: 46 - protein, 209 aa, Bacillus licheniformis
VGKEKTKKPIYKKWWFWLIIVIIIGAAASNGGNSEQASSTNKEKSTESKTTETKQDTKKEEKKEEPKKEETNPKIGDDVKVGDMNYKITGKKTADQVGPSALPQKASDKYLVIDVTLKNNGNEKVTVDASFFKLKRGEKTYEADSAASMSANQSEDGNIDNNFFLQNLNPDSKISGKVVFDVAPEVANAKDLQLQVQTGAWGTETGIID

SEQ ID NO: 47 - DNA, 1877 bp, Bacillus licheniformis
  source 1..1877: mol_type = other DNA
atgcaattagagatcgggaagcaatcgcagcaaaaccgtcacacgttgcaatttgaaaattggaggcagcacggggaattgatagctgcgcttttgtcgggtttgttgattcttgcaggctggctgttgtccggcaatgaaacattgtccgttgttctgtttattttagctttttgtatcggcggctttgctaaagcgaaagaaggtatacaagaaacgctgtcggaaaaaacgctgaatgttgaactcttgatgatttttgcggcggtcgggtctgcgctgatcggctactgggctgaaggggccgtgttaatttttatcttttctctcagcggagcgcttgaaacgtatacattaaataaaagcaaacgcgatttgacttcactgatgaaattggagccggaggaagccgttttgcttgaaaaagaaggaacaagaaccgtggcggcagccgatcttcaggcgggcgacctcattcttgtgaagcctggagaacgcattgcggcagacggagaaatcgaaaccggaaaaacgagtatcgacgaatcggctctaacgggcgaatccatcccagccgaaaaaacactcggagacgcggtttttgcgggtacggtcaatttgagcggatcgctcacggtccgtgtcacaaaggcaaatgaagattcgttgttcaaaaagatcattcggctcgttgagtctgcgcaaaacagcgtttcaccgtcgcaggcgttcattgagcgatttgaaaatatttacgttaaaggggttttgcttgctgtcggactgctgctctttctgccgcattttttgcttggctggagctggagcgagacattttacagagcgatggtgttcatggtcgtcgcttcgccgtgcgcccttgtcgcttcaatcatgcctgccgcgctgtcgctgatatcaaatggcgcaagaaacggcctgcttgtcaaaggcagcgtctttcttgaaaagcttggcaacagcaaaatcgttgcattggataaaacgggaacgattacgaacggaaagcccggcgtggaggatatgcttttagctgcagatatcgaggagcgcgaatgcttggaggctgctgctgcgattgagaagcagtcaggccatccgcttgccaaagcgattgtcgagtatgcagaagcaaaaggcatcaagccggccgcgaatgtgtcgattgaggagacgtcagggtttggcgttcaggctcgatacaatggagagacatggctgatcggcaaagccggatttgtcggggaagaggcagccgggcaatttctcacggcggctgttcaggagcttgccaagcagggaaaaacaattgtgttcatgaaaaaaggcgaaaaaatcgcggggtgttttgcattaaaagaccaaatcagaccggaagcaaaagcggtggtcgaagagctgaacgcgcttggcgttcagacggcgatgctgacaggggaccagcctgaaaccgcagcggccatcgcacgagaagcgggcttgaaaatcgtcgtttcagaatgccttcctgacagaaaagtagaggaagcgaaaaagctgaaaaaaacatacggaacaatcgttatggtgggagacggaatcaatgatgcaccggccctagccgccgctgatgtggggatcgcaatggggggaggaacagatgtcgcactggaaacggctgacgtcgttttaatgaaaaacgaattgacaggactgaccaaaatgattcggctctcccggaaaatgaatacgatcattaaacaaaacgtcattttctcgcttgctgtgatctgtcttttaatctgcagcaactttcttcaaattcttgatctgccgctcggtgtcatcgggcatgaaggcagcacgctgcttgtgat

SEQ ID NO: 48 - protein, 625 aa, Bacillus licheniformis
MQLEIGKQSQQNRHTLQFENWRQHGELIAALLSGLLILAGWLLSGNETLSVVLFILAFCIGGFAKAKEGIQETLSEKTLNVELLMIFAAVGSALIGYWAEGAVLIFIFSLSGALETYTLNKSKRDLTSLMKLEPEEAVLLEKEGTRTVAAADLQAGDLILVKPGERIAADGEIETGKTSIDESALTGESIPAEKTLGDAVFAGTVNLSGSLTVRVTKANEDSLFKKIIRLVESAQNSVSPSQAFIERFENIYVKGVLLAVGLLLFLPHFLLGWSWSETFYRAMVFMVVASPCALVASIMPAALSLISNGARNGLLVKGSVFLEKLGNSKIVALDKTGTITNGKPGVEDMLLAADIEERECLEAAAAIEKQSGHPLAKAIVEYAEAKGIKPAANVSIEETSGFGVQARYNGETWLIGKAGFVGEEAAGQFLTAAVQELAKQGKTIVFMKKGEKIAGCFALKDQIRPEAKAVVEELNALGVQTAMLTGDQPETAAAIAREAGLKIVVSECLPDRKVEEAKKLKKTYGTIVMVGDGINDAPALAAADVGIAMGGGTDVALETADVVLMKNELTGLTKMIRLSRKMNTIIKQNVIFSLAVICLLICSNFLQILDLPLGVIGHEGSTLLV

SEQ ID NO: 49 - DNA, 178 bp, Bacillus licheniformis
  source 1..178: mol_type = other DNA
atggaacgcttttcagaagaagagagaaaactgttgctgaacgtacttttggaccatgagtatgccgtagagctactaagcagtgagatcaatgatatagaaactggtacaaaaaatgtggatagcctgacatataagaaactggttaccttatatgaccgtgtccggtctgaaaatt

SEQ ID NO: 50 - protein, 59 aa, Bacillus licheniformis
MERFSEEERKLLLNVLLDHEYAVELLSSEINDIETGTKNVDSLTYKKLVTLYDRVRSEN

SEQ ID NO: 51 - DNA, 537 bp, Bacillus licheniformis
  source 1..537: mol_type = other DNA
atgattaatcagcttaaattgcgtccgcttgaaagagaagaccttccgtttgtccaccgtcttaacaacgatgcgaaaattatgtcatattggtttgaagaaccgtacgagacttttgttgagctgcaggatttatttgacaaacacattcacgaccaaagcgagcggcgctttatcatagagaaagagactgagatgatcggattggtagagctggtcgaaattgattatattcacaggcgggcggagtttcaaatcataattgatcccgagcatcaagggaacggttattcgtcaagcgcaacatatttggcaatgaactacgcattttccgtcttgaacttgcacaaattgtatttgatcgtcgacgaagataatgcaaaagcgattcacttgtataaaaaggcagggttcactatcgagagcgagctgcaggatgaatttttcgtcgacggctattatcgtaacgccattagaatgtgcatttttcaggatgagtttttatcacttaaaaaaagcaaagaggaaggcatgcag

SEQ ID NO: 52 - protein, 178 aa, Bacillus licheniformis
MINQLKLRPLEREDLPFVHRLNNDAKIMSYWFEEPYETFVELQDLFDKHIHDQSERRFIIEKETEMIGLVELVEIDYIHRRAEFQIIIDPEHQGNGYSSSATYLAMNYAFSVLNLHKLYLIVDEDNAKAIHLYKKAGFTIESELQDEFFVDGYYRNAIRMCIFQDEFLSLKKSKEEGM

SEQ ID NO: 53 - DNA, 207 bp, Bacillus licheniformis
  source 1..207: mol_type = other DNA
atgcaccatatgaacgtatcatacttaagaaattgttttgccgagatgaaacagtatgagacagactgcatgaacaaactgatggatttcgctaagtttttgtatatccagggacatctgacattaaacgaatttcgcacaagtatgaaagttcttgaagcgaatggcgcacagcaccctgcttatgatatgaatacggacgccagc

SEQ ID NO: 54 - protein, 69 aa, Bacillus licheniformis
MHHMNVSYLRNCFAEMKQYETDCMNKLMDFAKFLYIQGHLTLNEFRTSMKVLEANGAQHPAYDMNTDAS

SEQ ID NO: 55 - protein, 69 aa, Bacillus licheniformis; identical to SEQ ID NO: 54

SEQ ID NO: 56 - protein, 160 aa, Bacillus licheniformis
MKKAILGFSLSAALLVPSSFAAAAGDGQLTSEQKELLEAKTEYVQSLPEQASVQSGVTAYAGKRLTIKRGSFLAWSKDYIDWYYNGKKVSKSSGSQDVGYVFPNVVRAKGIKRYYKSSGLHKWRAKKTLSFGTVTPWGDVELASSTFTDRRWVNKKGKYG

SEQ ID NO: 57 - DNA, 422 bp, Bacillus licheniformis
  source 1..422: mol_type = other DNA
atgacaacaccgagtatggaagattacatagaacaaatttatatgctgattgaagaaaaaggatatgcaagagtctcagatatagccgaagctctggccgtccatccctcctcggttacaaaaatggttcaaaaactcgataaagacgaatatttgatttatgaaaagtatcgcggcctcgtgctgacgcctaaaggaaagaaaataggcaagcgtttagtatacagacatgaattattggagcagtttttacgaatcattggtgttgacgaagagaaaatttatgatgatgttgaaggaatcgaacatcatttaagctggaacagcattgaccgtatcggagatcttgtgcagtattttgaagacgacagcaaaagaatcgacgatttgagaagcgtccaaaaaagaaacgaacaggaaaa

SEQ ID NO: 58 - protein, 140 aa, Bacillus licheniformis
MTTPSMEDYIEQIYMLIEEKGYARVSDIAEALAVHPSSVTKMVQKLDKDEYLIYEKYRGLVLTPKGKKIGKRLVYRHELLEQFLRIIGVDEEKIYDDVEGIEHHLSWNSIDRIGDLVQYFEDDSKRIDDLRSVQKRNEQE

SEQ ID NO: 59 - DNA, 234 bp, Bacillus licheniformis
  source 1..234: mol_type = other DNA
gtgccggacggttcaccatatcgtaaacgctatcaaccagccggtccggtgcggcagatgaaaaacaaacgtctgttcatactttcgttgattgtgttgagcgtggtcatgatcggagttcaggaaaccttcccaaacctgctgacgacagggttaatgactgtcgttattgcggcggcagtttttaatattttaaaagaatcgctgaataaaaaagatgaaacacacagcaaa

SEQ ID NO: 60 - protein, 78 aa, Bacillus licheniformis
MPDGSPYRKRYQPAGPVRQMKNKRLFILSLIVLSVVMIGVQETFPNLLTTGLMTVVIAAAVFNILKESLNKKDETHSK

SEQ ID NO: 61 - DNA, 210 bp, Bacillus licheniformis
  source 1..210: mol_type = other DNA
atggctcaaaacaacagacaaagcagttctaaccaactattggttcctggtgctgctcaagctatcgaccaaatgaaattcgaaatcgcttctgaatttggcgttaaccttggagcagaaactacttctcgtgcaaacggttcagttggaggagaaatcactaagcgtttagtttctttcgctcaacagcaaatgggtggaacacaacaa

SEQ ID NO: 62 - protein, 70 aa, Bacillus licheniformis
MAQNNRQSSSNQLLVPGAAQAIDQMKFEIASEFGVNLGAETTSRANGSVGGEITKRLVSFAQQQMGGTQQ

SEQ ID NO: 63 - DNA, 1502 bp, Bacillus licheniformis
  source 1..1502: mol_type = other DNA
atgagcaacactgaacttgatttgctgaggcagcaggcgaatgaattaaacttgcaaatattaaagctgattaacgagcgaggaagaatcgttcaggagattggaaaagcgaaggaagcacaaggcatcaaccgctacgatcctgtcagagaaagagcgatgctgaacgaaattattgaaaataacgacggaccgtttgaaaactcgacgatccagcatatcttcaaagagattttcaaagccgggcttgaactgcaggaagacgatcacagcaaagcgctgctcgtttcccgcaagaaaaagcctgaaaatacaatcgttgatctgaaaggcgaaaaaatcggcgacggagaacaaagatttatcgtcggtccgtgtgcggttgaaagctacgaacaagtagcggaagtcgcagcggcagctaaaaagcaaggcttgaaactgcttcgcggcggagctttcaaaccgcgtacaagcccgtacgacttccaaggcctcggcgtggaaggcctgcaaatcttaaagcgtgttgctgatgagtatgatctggccgtcatcagtgagatcgtgaatccgcagcatattgaagaagccattgattacatcgatgtcatccaaatcggcgcccgcaacatgcagaacttcgagctcttaaaagcggccggttcagtgaagaagccggttctgctgaagcgcggacttgccgcaacgctgaaggaattcatcaatgcagcggagtacatcatgtcgcagggcaatgatcaaatcatcctttgtgaacgcggaatcagaacgtatgaaacagcgactagaaatacgcttgatatctcagctgtgccgatcctgaagcaggaaactcaccttcctgtattcgtggatgtcactcattcaaccggacgccgcgacctgcttcttccgacagcaaaagcagcgcttgcgatcggagctgacggtgtcatggccgaggtccatcctgatccttctgtcgcgctttccgactcagctcagcaaatggatatcccgacatttgaaaaatggttaaatgaactgaagccgctcgttcaagtaaaagcataattgaacactgaattaaagtacatgcttcaatccgttctaaaagaagaattgagtccgatcaatcagcgccttgatggaatcgacaagcgcttagacaaaatcgacgcacgcttcgttgagatagacaagcgcttcgatgaagtggacgcacgcttcgttgaagtagataagcgctttaacgcaatcgacaagcgcttcaaagaaatagacgggcgcttaaacaaagtagaaaaccgcttaaacgcaatggacaagcggctgaatcggcttgaaacagatatagatgaactgaaaagagggcaggagaggctcccgaaaacgatcatcgagaacatcggacaatttacggagaacattgtcgaacatgccgatgataaggcggctgccttgaatgacagggtttttagcgtcgaaaccgcaataca

SEQ ID NO: 64 - protein, 147 aa, Bacillus licheniformis
MNTELKYMLQSVLKEELSPINQRLDGIDKRLDKIDARFVEIDKRFDEVDARFVEVDKRFNAIDKRFKEIDGRLNKVENRLNAMDKRLNRLETDIDELKRGQERLPKTIIENIGQFTENIVEHADDKAAALNDRVFSVETAIQRIYRL

SEQ ID NO: 65 - DNA, 375 bp, Bacillus licheniformis
  source 1..375: mol_type = other DNA
atgaaactaaattatgattgtgtccgctcaattcttctagaattagaggaaaatttaactcttaacgatggcgtcactttatatcagctcaaagattttgagacattcaaagagtatggctatgaaacttccgtttacgctttaaccaagttaatcgaagctgactttttaaacggttcggtttcgcgcgcagacaataagattgactatattggtgttggctctattacttgggatggacatcaatttttagacaacattcgtgacaatgctgtttggtctaaaacgaaagatgccgttaagtcgttatcaagcgtttccttgtccatactttcaaatgtcggagaaagcatcacgaaaaagcttatcggttta

SEQ ID NO: 66 - protein, 124 aa, Bacillus licheniformis
MKLNYDCVRSILLELEENLTLNDGVTLYQLKDFETFKEYGYETSVYALTKLIEADFLNGSVSRADNKIDYIGVGSITWDGHQFLDNIRDNAVWSKTKDAVKSLSSVSLSILSNVGESITKKLIG

SEQ ID NO: 67 - DNA, 458 bp, Bacillus licheniformis
  source 1..458: mol_type = other DNA
atgatctctcaacaattaaaacagcaaaatcctgtacttgaaaactcaatgaatacgaatttgtcaaactggttcatcctttacacgaagcttcaccgttttcactggtacgtaaaagggccgcagttctttaccctacatgaaaaatttgaagagctatacaaccatgcgtcagaaacagcagatgtgatcgcggaacgcttgctggccatcggcggacagccgctcgccacaatgaaagaatacattgaccacggcacaattgaggaaaacggagccgaaaaaacggccgaagaaatggtttccgcactggtaagcgattaccgtcaaatccgcgatgaaattcagcataccatcgaacttgcggaagataaatcagatcattctaccgctgacctttatatcgccctgacagaggaaattgataagcagatttggatgctttcttcatttttgga

SEQ ID NO: 68 - protein, 152 aa, Bacillus licheniformis
MISQQLKQQNPVLENSMNTNLSNWFILYTKLHRFHWYVKGPQFFTLHEKFEELYNHASETADVIAERLLAIGGQPLATMKEYIDHGTIEENGAEKTAEEMVSALVSDYRQIRDEIQHTIELAEDKSDHSTADLYIALTEEIDKQIWMLSSFL

SEQ ID NO: 69 - DNA, 373 bp, Bacillus licheniformis
  source 1..373: mol_type = other DNA
atgaatgagaagattttaatcgtagacgaccagtacgggattcgggttctgctgaatgaagttttcaataaagaagggtacaaaaccttccaggccgcaaacgggattcaagcgcttgacattgtgaaaaaccagcgccccgacctcgttctgctcgatatgaaaatccccggaatggacggaattgaaattttaaaaagaatgaagatcatagacgaggggatccgcgtcatcatcatgacggcctacggagagctcgacatgatccaggaatcgaaggagctcggagccctgacacactttgctaagccttttgacatcgacgaaatccgcgatgccgtcaaaacgtatctgcccataaagtccaacggat

SEQ ID NO: 70 - protein, 122 aa, Bacillus licheniformis
MNEKILIVDDQYGIRVLLNEVFNKEGYKTFQAANGIQALDIVKNQRPDLVLLDMKIPGMDGIEILKRMKIIDEGIRVIIMTAYGELDMIQESKELGALTHFAKPFDIDEIRDAVKTYLPIKS

SEQ ID NO: 71 - DNA, 523 bp, Bacillus licheniformis
  source 1..523: mol_type = other DNA
ttgcttaaaattttcaccactcaattgacgggaatcttcaaccggattcaagagggcgaggcccaatcgatagaggacggcgcccgcttgttggctcaggcggtcatcagtgatcattcggtttatgtatacggaaaaaatgaattggaaggcattttgaaagaagcgatgtacagttctgaaccgttcccctcagtcaaaccgctgcagaaggacgaagaaaactggcccgattttgaaatgacggacaaagtgctgatgttctgcgccggttcagcggatgacgaagaactgaaaatggcggaaaaactgtatgaaaaaggaatcggacttgttgtcgtctccccttcgggaaaagacggcgtcccgatcgcatccttcgccgatgtccatatcgattcaaagctgagaatgccgcttcttcccgatgaagacggaacaaggtacggatttccgtccctgatggtcagcctctatatttatcatgccctatcctttacattaaaggaaatcctccaggaat

SEQ ID NO: 72 - protein, 174 aa, Bacillus licheniformis
MLKIFTTQLTGIFNRIQEGEAQSIEDGARLLAQAVISDHSVYVYGKNELEGILKEAMYSSEPFPSVKPLQKDEENWPDFEMTDKVLMFCAGSADDEELKMAEKLYEKGIGLVVVSPSGKDGVPIASFADVHIDSKLRMPLLPDEDGTRYGFPSLMVSLYIYHALSFTLKEILQE

SEQ ID NO: 73 - DNA, 266 bp, Bacillus licheniformis
  source 1..266: mol_type = other DNA
atgagcaaaacgattaaacttaaccatgcagcggttatgaagaagctggaacaagtaagcagcacgcttcaagccgtatctttaaaaagcccgccagccggagcgctcgggcggaacaaccttgattttacgaaaaagtggcttgaacgagaagccgaaatttgcaatatggtcaaacagtataaagaggctgttcgtaaaaacattgaggacacccgctcaaatgtggacacgctgaaggaacaggatgaggcgattgcccgaag

SEQ ID NO: 74 - protein, 88 aa, Bacillus licheniformis
MSKTIKLNHAAVMKKLEQVSSTLQAVSLKSPPAGALGRNNLDFTKKWLEREAEICNMVKQYKEAVRKNIEDTRSNVDTLKEQDEAIAR

SEQ ID NO: 75 - DNA, 1396 bp, Bacillus licheniformis
  source 1..1396: mol_type = other DNA
atgaaatataaaacattagctcaatttccgaaagattttttgtggggcgcgtctacttccgcttatcaagtggaaggcgcttgggacgaagatggaaaagggccttctgtcatcgatgcgcgcgaaagctacccggaagggacaaccgattttaaagtcgcaagcgaccattaccagcgctataaagaagatatcgctttatttgcggaaatgggcttcaaagcgtaccgtttttcgattgcctggacgcgcatcattccggacggggacggtgatattaatccgaagggaatcgaattttacagccgtttgatcgatgaacttctaaagtatggaatcgaaccaattgttacgatgtaccactttgatctgccgaatgctttgcagaaaaaaggcggctggtcggacagggccacgattgatgcttttgaaaagtatgcgaaggtcctttttgaaagctacggtgaccgcgtcaaatactggctgaccatcaatgagcaaaatatgatgatcctccacggatctgcactcggtacactcgatccgaacttggaaaatccgaaaaaagagctttatcagcaaaaccatcacatgctcgtcgcacaggcgaaagcgatcaagctttgccatgagatgctgccggaagcaaaaatcggtcctgcgccgaatattgcgctcatctatcccgcttcttcgaaaccggaggacgtgctggcggcttttaactataatgcgatccgaaactggctttacttggatatggccgtattcggacggtacaatacaacagcgtgggcatatatgaaagaaaaaggctgcacaccggtcatcgctgaaggggatatggacattctgcggtcggccaagccggattttatcgcgtttaactactatacatcgcaaacggctgaagcaagcaggggtgatggcagcgacacggctgctcgaggcggagaccagcatttgcagacgggagaagaaggcgtatataggggaagcagcaatccgcacctaaagaaaaacgcatttggctgggagatcgaccctgtcggtttccgttcgacgctgcgcgaaatttacgaccgctaccagctgccgctgatcgtcactgagaacggcctcggcgcgtttgatcagcttgaagacggagatgtcgtaaatgacgattaccgcatcgattatttaaaagagcatatcaagcaaattcagctggcaatcacggatggagtcgatgttttcggctactccccatggtctgccatcgacttaatttcgacccatcaaggctgttcaaaacgctacggatttatttatgtgaaccgcgatgaatttgatttgaaagacttgcgccgcattcgcaaaaaaagcttttactggtataaaaacctgattgcta

SEQ ID NO: 76 - protein, 465 aa, Bacillus licheniformis
MKYKTLAQFPKDFLWGASTSAYQVEGAWDEDGKGPSVIDARESYPEGTTDFKVASDHYQRYKEDIALFAEMGFKAYRFSIAWTRIIPDGDGDINPKGIEFYSRLIDELLKYGIEPIVTMYHFDLPNALQKKGGWSDRATIDAFEKYAKVLFESYGDRVKYWLTINEQNMMILHGSALGTLDPNLENPKKELYQQNHHMLVAQAKAIKLCHEMLPEAKIGPAPNIALIYPASSKPEDVLAAFNYNAIRNWLYLDMAVFGRYNTTAWAYMKEKGCTPVIAEGDMDILRSAKPDFIAFNYYTSQTAEASRGDGSDTAARGGDQHLQTGEEGVYRGSSNPHLKKNAFGWEIDPVGFRSTLREIYDRYQLPLIVTENGLGAFDQLEDGDVVNDDYRIDYLKEHIKQIQLAITDGVDVFGYSPWSAIDLISTHQGCSKRYGFIYVNRDEFDLKDLRRIRKKSFYWYKNLIA

SEQ ID NO: 77 - DNA, 1830 bp, Bacillus licheniformis
  source 1..1830: mol_type = other DNA
atgaatgttcaatcaatggcaaaagaaattctggcgcgcgtcggcggggagaagaacgttgtatcgctagtccattgcgcgacaaggctccgcttcaaactgaaagaccgttccaaagcagacagagaagcgcttgaaaatacggatggcgtggtaaccgtggtggagagcggcgggcaatttcaagtcgttatcggcaataacgttcctgaagtatataaagaaatcggccggatctctcaactgttggaaagttctgcggccgatcacagtcaaaaagagaaaggaagcctcgccggtagattagtagatgtcgtatccagcattttcactccgctgctcggcgtcatggcgggagcggggattttaaaaggcctgcttttgatttgcacaaacgccggctggctctcgccggaagagacgacatatacgattctatacgcggcagccgacagcctgttttatttcctgcctttgctgctcgccgttacggccgccaaaaaattcggagcgaacccgttcatagcgctgacgattgccggagcgctgatctatccgacgatactcgaactgaaaaacagcggcgcccatacggagtttttcggcattccggtcgtcttgatgaattatacgtcgaccgttatcccgattatccttgcggtatttgtgatgagctatcttgaaagattatgtatgcgctttattcatgaaagcgtgaagaactttatcacccctttattatgcctgacggtgatggtgccgttaactttgatcgtattcggaccgctcggcgtttacacaggaaacgggatcgcggcggcgattctgtccgtttttgatttcagcccgattctggcaggagcgattattgcggccttatggcaaatccttgtcatcttcgggattcactgggggatcgtccctgtcattttgaacaacattgccgtccacggcaaggactacattaagccggcgacggcagccgccgtgtttgcccaaacaggagccgcatttggcgtcatgctgaagacgaaaaacaagaagctgaaagcattggcaggatcagctgccgttaccgggatattcgggattacagagccggccgtctacggggtgacgctcaggctgaaaaagccgtttgtatgcggtgtcatcagcgcggcggcaggcggagccattatcggatactccggcagcgtcgccctcgcttccggcgctccgggcttgttgacgattccgattttttacggaccgggctttctcggcttcatcatcggcatttcagtttcatttgttttatcgattttgctcacttacattgtcggctttgacgaccctgtggaggtgacaaaagaggagcctgttgaaaaaagcgccggtgaaccgatttacagccctttgcaaggagaagtcctgccgttaacagaagtatcagataaagtgttcgcatcgggggcgcttggagagggaatcgcggtggttccgtcaaaaggcgttgtcaccgcccctgcggacggcatcgtgacaacggcatttccgacgggtcacgcatacggcataacgaccgaaagcggagccgagattttgattcatattggaatggacacagtcaggctcgaaggaaagcacttcaatcctaaagccgtgcaggggcaaaatgtgaaacggggtgatatactcgcagaattcgatctggacgctttgaaagaagaagggtttgacgtgaaaacgccgatcatcgttacaaattccggtcaatacacggatgtcattccaacggatcaaaaaaaggtgaaaactgaagaacggatcatcactttaatttcatca

SEQ ID NO: 78 - protein, 609 aa, Bacillus licheniformis
MNVQSMAKEILARVGGEKNVVSLVHCATRLRFKLKDRSKADREALENTDGVVTVVESGGQFQVVIGNNVPEVYKEIGRISQLLESSAADHSQKEKGSLAGRLVDVVSSIFTPLLGVMAGAGILKGLLLICTNAGWLSPEETTYTILYAAADSLFYFLPLLLAVTAAKKFGANPFIALTIAGALIYPTILELKNSGAHTEFFGIPVVLMNYTSTVIPIILAVFVMSYLERLCMRFIHESVKNFITPLLCLTVMVPLTLIVFGPLGVYTGNGIAAAILSVFDFSPILAGAIIAALWQILVIFGIHWGIVPVILNNIAVHGKDYIKPATAAAVFAQTGAAFGVMLKTKNKKLKALAGSAAVTGIFGITEPAVYGVTLRLKKPFVCGVISAAAGGAIIGYSGSVALASGAPGLLTIPIFYGPGFLGFIIGISVSFVLSILLTYIVGFDDPVEVTKEEPVEKSAGEPIYSPLQGEVLPLTEVSDKVFASGALGEGIAVVPSKGVVTAPADGIVTTAFPTGHAYGITTESGAEILIHIGMDTVRLEGKHFNPKAVQGQNVKRGDILAEFDLDALKEEGFDVKTPIIVTNSGQYTDVIPTDQKKVKTEERIITLIS

SEQ ID NO: 79 - DNA, 821 bp, Bacillus licheniformis
  source 1..821: mol_type = other DNA
gtgaaaattgccaaagtgatcaacaacaatgtgatcagtgttttaaatgaacagggtcaggaattagtggtcatgggcaggggaatcgcttttcaaaaaaagcctggcgaagcggtggatgaatcgagaatcgagaagattttcaggcttgataataaagatgtatcagaaaggtttaagacgctgttggacgaaattccaattgaatttatggaaatgtctgaagaaattatctcctatgcgaaattaaagctcggcaaaaagctgaatgacagcatttatgtctcgctgaccgaccatattcatttcgccgtcgaacggcataaaaagggactggatattaaaaatgccctgctttgggagacgaaacggctgtataaagatgagttcgccatcggcaaagaggcgctggccatcattgaaaagaagacggggacagctcttccggaggatgaagccgcctttatcgcgctccatatcgtaaacgccgagctgaatgaagaaatgccgaacatcgtcaatattacgaaagtcatgcaggagattttaagcatcgtcaagtatcactttcatatcgaatttgacgaagaatcgcttcactattaccgcttcattacccatttgaaatttttcgcccagcggctgttcagcggaacctatatggaaagccgggacgactttttatttgaaaccgtcaaaaataagtaccgggacgcttatgtttgtacaaataaaatcaggcagtatatcgaaaaagaatacggtcatcagctgacaaacgaagagcttctgtatttgacgattcatatcgagcgagtcgt

SEQ ID NO: 80 - protein, 273 aa, Bacillus licheniformis
MKIAKVINNNVISVLNEQGQELVVMGRGIAFQKKPGEAVDESRIEKIFRLDNKDVSERFKTLLDEIPIEFMEMSEEIISYAKLKLGKKLNDSIYVSLTDHIHFAVERHKKGLDIKNALLWETKRLYKDEFAIGKEALAIIEKKTGTALPEDEAAFIALHIVNAELNEEMPNIVNITKVMQEILSIVKYHFHIEFDEESLHYYRFITHLKFFAQRLFSGTYMESRDDFLFETVKNKYRDAYVCTNKIRQYIEKEYGHQLTNEELLYLTIHIERV

SEQ ID NO: 81 - DNA, 1387 bp, Bacillus licheniformis
  source 1..1387: mol_type = other DNA
atgactgaacaaacgaaaaagtttcctgaaggttttttatggggcggagcggttgccgcaaaccaagtggaaggcgcctataatgtcggcgggaaaggactctcgacagccgatgtgtcgccgaacggcgtcatgtatccattcgatgagtcgatggagtcattgaacctgtatcatgaaggcatcgatttttaccaccgctacaaagaagatatcgctcttttcgccgaaatgggatttaaagcattccggacatcgattgcctggacgcggatttttcctaacggcgatgaaactgaaccgaatgaagagggactcgaattttatgatcgcctctttgacgagctattaaaatacaatattgagccagtggtgacgatttcccactacgaaatgccgctcggtctgattaaaaaatacggcggctggaaaaaccggaaagtgatcgattgctatgagcattatgcgaaaacggttttcacccgctataaagaaaaagtaaaatactggatgacattcaatgaaatcaacatggttctgcatgcgccgtttacaggcggaggccttgtgtttgaagaaggcgaaaacaagttaaatgcgatgtaccaagcggcgcatcatctatttgtcgcaagcgctcttgccgttaaagcggggcacgacatcattccggacgctaaaatcggctgcatgatcgccgcaacgacgacatacccgatgacgccaaagccggaagacgtgctcgctgcgatggagaatgagagaagaacgctgtttttctcggatgtacaggcgcgcggggcttatccgggctatatgaagcgcttctttaaggaaaacggaattacgattgaaatggctgaaggtgatgaagacatcttaaaggaaaacaccgtcgactatatcggtttcagctactacatgtcaatggtcgcaagcacgagtccggaagacttggcaaaaacggaaggcaacctgctcggcggcgtcaaaaatccgtaccttgaatcgtccgaatggggctggcagatcgatcctaaagggatccgcattacgctgaatacattgtacgaccgctatcaaaagccgcttttcattgttgaaaacgggctcggcgccgtcgatgtcgtcgaagaagacggctccatccaggatgactacagaatcaactatttgcgcgatcatttaaaagaagtaagagaagccattgcagacggcgtcgacttaatcggctacacatcatggggcccgatcgacctggtcagcgcatccaccgccgaaatgaaaaagcgctacggctatatttacgtcgatcgtgacaatgaaggaaaaggcacgctttcaagaacgagaaagaaaagcttttactggtataagaaagtaatcg

SEQ ID NO: 82 - protein, 462 aa, Bacillus licheniformis
MTEQTKKFPEGFLWGGAVAANQVEGAYNVGGKGLSTADVSPNGVMYPFDESMESLNLYHEGIDFYHRYKEDIALFAEMGFKAFRTSIAWTRIFPNGDETEPNEEGLEFYDRLFDELLKYNIEPVVTISHYEMPLGLIKKYGGWKNRKVIDCYEHYAKTVFTRYKEKVKYWMTFNEINMVLHAPFTGGGLVFEEGENKLNAMYQAAHHLFVASALAVKAGHDIIPDAKIGCMIAATTTYPMTPKPEDVLAAMENERRTLFFSDVQARGAYPGYMKRFFKENGITIEMAEGDEDILKENTVDYIGFSYYMSMVASTSPEDLAKTEGNLLGGVKNPYLESSEWGWQIDPKGIRITLNTLYDRYQKPLFIVENGLGAVDVVEEDGSIQDDYRINYLRDHLKEVREAIADGVDLIGYTSWGPIDLVSASTAEMKKRYGYIYVDRDNEGKGTLSRTRKKSFYWYKKVI

SEQ ID NO: 83 - DNA, 1820 bp, Bacillus licheniformis
  source 1..1820: mol_type = other DNA
atggattataataaagtatcgaaggacattttacaactcgtgggcggtgaagagaacgtacagagcgtgattcactgcatgacaagactgcgtttcaatctttatgacaatgcgaaggcagaccgcgcgaaactggaaagtcttccggctgtaatgggcaccaacatcagcggccagcagtttcaaatcatcattggaaatgatgtccctaaggtgtataaagcgatcattgccaacagcgggctcagcgatgaaaaagcgggcgagcagcaagctggcaaaaagaaaaacgtgctgtccgccatttttgatgtgatatcaggcgtatttacgccgattctcccggcgatcgccggagcgggtatgatcaaggggatcattgcaatcgccgtcacgttcggctggatgagcgaaactagccaggtccacaccattttatccgccatcggcgacggagcattttacttcctgccgattttgctggcggtcagtgccgcgagaaaattcggcagtaatccttatgttgcggcggcgatcggagcggcgattctgcatcccgatctgacagcgcttctcggttcagggaaaagcatttcctttgtcggattgcctgtaaccgctgccacttattcgtcgaccgtcattccgatcctgctcgcgatttggattgcatcttacgttgagaaatggattgacaaagtcaccccgacttcgttgaagatgatttttgtaccgacgctgacgctgctggttgtcgtgcctgtcacattaattacggtcggtccgctcggtgcgattgcagggaactatctgtcaatcggtgtgaacggtttatttgaaaatgccggtttaatcacgatgatccttttagcaggaacgttctcgctgatcgtcatgacaggaatgcactatgctttcattccggttatgttcaacaacattacgcaaaacggctacgattacttgatacctgcgatgttcttggcgaatatggggcaggctggcgcgtcatttgccgtcttcctgcgatccagaaataagaaattcaaatcattgtcgctgacgacaagcatcacggctttgatggggattacagagccggcgatgtacggtgtcaacatgaggctgaaaaagccgtttgtatccgcgcttctcggcgccgctgtcggaggagcattttacggcatcaccggcgtagcagcatatattattggcggaaacgtcggtttgccgggaattacgacgtttatcggcccgacttttatacaggcaatgatcggtatcgtcatcgcgttctttgccgcaacggcgtttgcttttgtattggggtttgaagacattccttcagatgaagccgctgaacaaggagccgctccatctgaagcgggcgcgggagagatcattcaaagcccgctgaaaggcgaagtcaaagcattgagtgaagtggatgatgcgacattttctggagaagtcatgggaaaaggcgtcgccattgagcctgaagaaggcaaagtggtgtcgccggtatcaggcacgatcacaaccgtttttcaaacgaagcacgccctcggcattacaagcgacaatggggcggaaattattatccatatcggaatcgacacggtgaaattgaacggcgagcactttaccgtgcatgtcaataaaggcgacgctgtaaaaccgggagacgagcttgtctcatttgatatggatgcgattaaggatgcaggctatcagttgatcacgccggttatcatcacaaatacggaccggtaccagtcgatcaaacctttgaaatcagatgaaagcgttgacattga

SEQ ID NO: 84 - protein, 604 aa, Bacillus licheniformis
MDYNKVSKDILQLVGGEENVQSVIHCMTRLRFNLYDNAKADRAKLESLPAVMGTNISGQQFQIIIGNDVPKVYKAIIANSGLSDEKAGEQQAGKKKNVLSAIFDVISGVFTPILPAIAGAGMIKGIIAIAVTFGWMSETSQVHTILSAIGDGAFYFLPILLAVSAARKFGSNPYVAAAIGAAILHPDLTALLGSGKSISFVGLPVTAATYSSTVIPILLAIWIASYVEKWIDKVTPTSLKMIFVPTLTLLVVVPVTLITVGPLGAIAGNYLSIGVNGLFENAGLITMILLAGTFSLIVMTGMHYAFIPVMFNNITQNGYDYLIPAMFLANMGQAGASFAVFLRSRNKKFKSLSLTTSITALMGITEPAMYGVNMRLKKPFVSALLGAAVGGAFYGITGVAAYIIGGNVGLPGITTFIGPTFIQAMIGIVIAFFAATAFAFVLGFEDIPSDEAAEQGAAPSEAGAGEIIQSPLKGEVKALSEVDDATFSGEVMGKGVAIEPEEGKVVSPVSGTITTVFQTKHALGITSDNGAEIIIHIGIDTVKLNGEHFTVHVNKGDAVKPGDELVSFDMDAIKDAGYQLITPVIITNTDRYQSIKPLKSDESV

SEQ ID NO: 85 - DNA, 570 bp, Bacillus licheniformis
  source 1..570: mol_type = other DNA
atgagcacagaggatatgacaaaggatacgtatgaagtaaacagttcgacaatggctgtcctgcctctgggagagggggagaaacccgcctcaaaaatacttgagaccgacaggactttccgcgtcaatatgaagccgtttcaaattatcgaaagaagctgccgctatttcggatcgagctatgcgggaagaaaagcgggcacatatgaagtcattaaagtttcccataaaccgccgatcatggtggatcactcaaacaacatttttcttttccccacattttcctcaactcgtcctcagtgcgggtggctttcccatgcgcatgttcacgagttttgcgcggcaaagtatgacaacacgtttgtcacgtttgtcaacggggaaacgctggagctgcccgtatccatctcatctttcgaaaaccaggtttaccgaacggcatggctgagaacaaaatttatcgacaggattgaaggaaaccccatgcagaagaaacaggaatttatgctctatccgaaagaagaccggaatcagctgatatacgaattcatcctcagggagctgaaaaag

SEQ ID NO: 86 - protein, 189 aa, Bacillus licheniformis
MSTEDMTKDTYEVNSSTMAVLPLGEGEKPASKILETDRTFRVNMKPFQIIERSCRYFGSSYAGRKAGTYEVIKVSHKPPIMVDHSNNIFLFPTFSSTRPQCGWLSHAHVHEFCAAKYDNTFVTFVNGETLELPVSISSFENQVYRTAWLRTKFIDRIEGNPMQKKQEFMLYPKEDRNQLIYEFILRELK

SEQ ID NO: 87 - DNA, 36 bp, Bacillus licheniformis
  source 1..36: mol_type = other DNA
gccgcagccatttccagagccgcagccatttccaga

SEQ ID NO: 88 - protein, 1368 aa, Streptococcus pyogenes [sequence omitted]

SEQ ID NO: 89 - protein, 1307 aa, Acidomiococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 90 - protein, 887 aa, N. gregoryi
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL

SEQ ID NO: 91 - DNA, 12203 bp, Streptococcus pyogenes
  source 1..12203: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaac

SEQ ID NO: 92 - DNA, 4107 bp, synthetic construct
  misc_feature 1..4107: note = codon optimized S. pyogenes Cas9
  source 1..4107: mol_type = other DNA
atggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagactaa

SEQ ID NO: 93 - DNA, 607 bp, Bacillus subtilis
  source 1..607: mol_type = other DNA
attcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaacta

SEQ ID NO: 94 - DNA, 138 bp, Bacillus subtilis
  source 1..138: mol_type = other DNA
tattgaaaatactgacgaggttatataagatgaaaataagttagtttgtttaaacaacaaactaataggtgatgtacttactatatgaaataaaatgcatctgtatttgaatgaatttatttttaagggggaaatcac

SEQ ID NO: 95 - DNA, 177 bp, synthetic construct
  misc_feature 1..177: note = spac promoter
  source 1..177: mol_type = other DNA
ggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcgctcacaatt

SEQ ID NO: 96 - DNA, 100 bp, synthetic construct
  misc_feature 1..100: note = hyper-spank promoter
  source 1..100: mol_type = other DNA
ctcgagggtaaatgtgagcactcacaattcattttgcaaaagttgttgactttatctacaaggtgtggcataatgtgtgtaattgtgagcggataacaat

SEQ ID NO: 97 - DNA, 237 bp, Bacillus subtilis
  source 1..237: mol_type = other DNA
ggagttctgagaattggtatgccttataagtccaattaacagttgaaaacctgcataggagagctatgcgggttttttattttacataatgatacataatttaccgaaacttgcggaacataattgaggaatcatagaattttgtcaaaataattttattgacaacgtcttattaacgttgatataatttaaattttatttgacaaaaatgggctcgtgttgtacaataaatgtagt

SEQ ID NO: 98 - DNA, 270 bp, Bacillus subtilis
  source 1..270: mol_type = other DNA
gaattcgagctcggtaccgatcttaacatttttcccctatcatttttccgtcttcatttgtcattttttccagaaaaaatcgcgtcattcgactcatgtctaatccaacacgtgtctctcggcttatcccctgacaccgcccgccgacagcccgcatgggacgattctatcaattcagccgcggagtctagttttatattgcagaatgcgagattgctggtttattataacaatataagttttcattattttcaaaaagggggatttatt

SEQ ID NO: 99 - DNA, 56 bp, T5 phage
  source 1..56: mol_type = other DNA
aagaatcataaaaaatttatttgctttcaggaaaatttttctgtataatagattca

SEQ ID NO: 100 - DNA, 173 bp, Bacillus subtilis
  source 1..173: mol_type = other DNA
gctattgtaacataatcggtacgggggtgaaaaagctaacggaaaagggagcggaaaagaatgatgtaagcgtgaaaaattttttatcttatcacttgaaattggaagggagattctttattataagaattgtggaattgtgagcggataacaattcccaattaaaggaggaa

SEQ ID NO: 101 - DNA, 231 bp, Bacillus subtilis
  source 1..231: mol_type = other DNA
aagcttctcatcaatgatttgaattggagctcgggctggccgtcctattgaattaaaaagccgggctctgcccccggctttttttaaaagaaaagattgacagtataatagtcaattactataataaaattgttcgtacaaatatttatttataggtttattttctatcattagtacgtatcttttgtatttgaaagcgttttattttatgagaaaggggcagtttacatg

SEQ ID NO: 102 - DNA, 231 bp, Bacillus licheniformis
  source 1..231: mol_type = other DNA
ttctgaaatctgtcaaaaaaaataaaaaacataccggaaattaaattgacagtttttttcataatgatataatgaagttgttcgtacaaatatgtttttgatgttagttgtacgtacatataatcgcgatacagtttgagatcaaggtatgatttatgtttttttgtaagcgttttaatagtttgctattctacacagacaccataaagacgaggaggaggaagctatttg

SEQ ID NO: 103 - DNA, 95 bp, lambda phage
  source 1..95: mol_type = other DNA
gactcctgttgatagatccagtaatgacctcagaactccatctggatttgttcagaacgctcggttgccgccgggcgttttttattggtgagaat

SEQ ID NO: 104 - DNA, 4809 bp, synthetic construct
  misc_feature 1..4809: note = cas9 expression cassette
  source 1..4809: mol_type = other DNA
attcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaactaatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagactaagactcctgttgatagatccagtaatgacctcagaactccatctggatttgttcagaacgctcggttgccgccgggcgttttttattggtgagaat

SEQ ID NO: 105 - DNA, 23 bp, Bacillus licheniformis
  source 1..23: mol_type = other DNA
aatggctgcggctctggaaatgg

SEQ ID NO: 106 - DNA, 17 bp, synthetic construct
  misc_feature 1..17: note = 17 bp VT
  source 1..17: mol_type = other DNA
ggctgcggctctggaaa

SEQ ID NO: 107 - DNA, 18 bp, synthetic construct
  misc_feature 1..18: note = 18bp VT
  source 1..18: mol_type = other DNA
tggctgcggctctggaaa

SEQ ID NO: 108 - DNA, 19 bp, synthetic construct
  misc_feature 1..19: note = 19bp VT
  source 1..19: mol_type = other DNA
atggctgcggctctggaaa

SEQ ID NO: 109 - DNA, 20 bp, synthetic construct
  misc_feature 1..20: note = 20bp VT
  source 1..20: mol_type = other DNA
aatggctgcggctctggaaa

SEQ ID NO: 110 - DNA, 76 bp, synthetic construct
  misc_feature 1..76: note = Cas9 endoncuclease recognition domain
  source 1..76: mol_type = other DNA
gttttagagctagaaatagcaagttaaaataaggctagtccgttatcaacttgaaaaagtggcaccgagtcggtgc

SEQ ID NO: 111 - DNA, 96 bp, synthetic construct
  misc_feature 1..96: note = DNA encoding gRNA targeting duplication
  source 1..96: mol_type = other DNA
aatggctgcggctctggaaagttttagagctagaaatagcaagttaaaataaggctagtccgttatcaacttgaaaaagtggcaccgagtcggtgc

SEQ ID NO: 112 - DNA, 368 bp, synthetic construct
  misc_feature 1..368: note = duplication gRNA expression cassette
  source 1..368: mol_type = other DNA
ggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcgctcacaattaatggctgcggctctggaaagttttagagctagaaatagcaagttaaaataaggctagtccgttatcaacttgaaaaagtggcaccgagtcggtgcgactcctgttgatagatccagtaatgacctcagaactccatctggatttgttcagaacgctcggttgccgccgggcgttttttattggtgagaat

SEQ ID NO: 113 - DNA, 500 bp, Bacillus licheniformis
  source 1..500: mol_type = other DNA
gtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccaga

SEQ ID NO: 114 - DNA, 500 bp, Bacillus licheniformis
  source 1..500: mol_type = other DNA
atcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcc

SEQ ID NO: 115 - DNA, 1000 bp, synthetic construct
  misc_feature 1..1000: note = rghR2 duplication editing template
  source 1..1000: mol_type = other DNA
gtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcc

SEQ ID NO: 116 - DNA, 20 bp, Bacillus licheniformis
  source 1..20: mol_type = other DNA
gcgaatcgaaaacggaaagc

SEQ ID NO: 117 - DNA, 20 bp, synthetic construct
  misc_feature 1..20: note = rghR2 locus forward
  source 1..20: mol_type = other DNA
gcgaatcgaaaacggaaagc

SEQ ID NO: 118 - DNA, 20 bp, synthetic construct
  misc_feature 1..20: note = rghR2 locus reverse
  source 1..20: mol_type = other DNA
tcatcgcgatcggcattacg

SEQ ID NO: 119 - DNA, 1146 bp, synthetic construct
  misc_feature 1..1146: note = Edited rghR2 locus
  source 1..1146: mol_type = other DNA
gcgaatcgaaaacggaaagcgcggcgtgccgaagccggcgacgatcagaaaactggcggacgctttgaaagtcccgtatgaggaactgatggcatctgcaggctatatcagcgcgtctacagtccaggaagcaagaagcagctatgattccatttacgacatcgtgtcacagtacgatttagaggacctttctctgtttgacagcgaaaagtggaaggtgctttcaaaaaaagacatcgaaaacctggacaaatatttcgactttctcgtgcaggaagcaagcagccgaaacaaaaactgaatacttctccgcggcacactctcctctctatcattttcgtctgtttacgatcctgctgttattttatcccttatgttaacttttgtcaatatttttcctgtctaagtatttcctatagtcaacatttgtattaaaatgttcatatcatgaatttgcgggggggatggcgatgacaaggttcggcgagcggctcaaagagctgagggaacaaagaagcctgtcggttaatcagcttgccatgtatgccggtgtgagcgccgcagccatttccagaatcgaaaacggccaccgcggcgttcccaagcccgcgacgatcagaaaattggccgaggctctgaaaatgccgtacgagcagctcatggatattgccggttatatgagagctgacgagattcgcgaacagccgcgcggctatgtcacgatgcaggagatcgcggccaagcacggcgtcgaagacctgtggctgtttaaacccgagaaatgggactgtttgtcccgcgaagacctgctcaacctcgaacagtattttcattttttggttaatgaagcgaagaagcgccaatcataaaaagccgaatttcccttttaggagaagttcggcttttttcggctgccttaagcggcatccggattcggcgtcttgcctttatgatgcttaacggggctcagcgcacgctcgagccatcccatgaacagatcggcgatgatcgccatcagcgccgtcgggatcgcgcctgctagaatgatcgctgttccgttggtcgcgtttgatcccctgacaatgatatccccgaggccgcctgcgccgacaaacgtgccgatggccgtaatgccgatcgcgatga

SEQ ID NO: 120 - DNA, 20 bp, synthetic construct
  misc_feature 1..20: note = rghR2 sequencing primer
  source 1..20: mol_type = other DNA
tttcgactttctcgtgcagg

SEQ ID NO: 121 - DNA, 23 bp, Bacillus licheniformis
  source 1..23: mol_type = other DNA
ctttccgatcacaagttgtccgg

SEQ ID NO: 122 - DNA, 23 bp, Bacillus licheniformis
  source 1..23: mol_type = other DNA
tttccgatcacaagttgtccggg

SEQ ID NO: 123 - DNA, 23 bp, Bacillus licheniformis
  source 1..23: mol_type = other DNA
aggcccggacaacttgtgatcgg

SEQ ID NO: 124 - DNA, 23 bp, Bacillus licheniformis
  source 1..23: mol_type = other DNA
gagttttaaatggcgaaaggagg

SEQ ID NO: 125 - DNA, 23 bp, Bacillus licheniformis
  source 1..23: mol_type = other DNA
ttaaatggcgaaaggaggcccgg

SEQ ID NO: 126 - DNA, 23 bp, Bacillus licheniformis
  source 1..23: mol_type = other DNA
gcagagttttaaatggcgaaagg

SEQ ID NO: 127 - DNA, 23 bp, Bacillus licheniformis
  source 1..23: mol_type = other DNA
acactcagcagagttttaaatgg

SEQ ID NO: 128 - DNA, 23 bp, Bacillus licheniformis
  source 1..23: mol_type = other DNA
aactctgctgagtgtcgccgggg

SEQ ID NO: 129 - DNA, 23 bp, Bacillus licheniformis
  source 1..23: mol_type = other DNA
aaaactctgctgagtgtcgccgg

SEQ ID NO: 130 - DNA, 23 bp, Bacillus licheniformis
  source 1..23: mol_type = other DNA
aaactctgctgagtgtcgccggg

SEQ ID NO: 131 - DNA, 23 bp, Bacillus licheniformis
  source 1..23: mol_type = other DNA
cattgaaatcggcgtatccccgg

SEQ ID NO: 132 - DNA, 23 bp, Bacillus licheniformis
  source 1..23: mol_type = other DNA
cggggatacgccgatttcaatgg

SEQ ID NO: 133 - DNA, 23 bp, Bacillus licheniformis
  source 1..23: mol_type = other DNA
aatcagcttgccattgaaatcgg

SEQ ID NO: 134 - DNA, 23 bp, Bacillus licheniformis
  source 1..23: mol_type = other DNA
ttcaatggcaagctgatttaagg

SEQ ID NO: 135 - DNA, 23 bp, Bacillus licheniformis
  source 1..23: mol_type = other DNA
gattgtgttcaatcatgtactgg

SEQ ID NO: 136 - DNA, 23 bp, Bacillus licheniformis
  source 1..23: mol_type = other DNA
attagaaaggggagtaatcatgg

SEQ ID NO: 137 - DNA, 23 bp, Bacillus licheniformis
  source 1..23: mol_type = other DNA
aaacataagagattagaaagggg

SEQ ID NO: 138 - DNA, 23 bp, Bacillus licheniformis
  source 1..23: mol_type = other DNA
gaaacataagagattagaaaggg

SEQ ID NO: 139 - DNA, 23 bp, Bacillus licheniformis
  source 1..23: mol_type = other DNA
agaaacataagagattagaaagg

SEQ ID NO: 140 - DNA, 1748 bp, synthetic construct
  misc_feature 1..1748: note = amylase expression cassette
  source 1..1748: mol_type = other DNA
tcgctgataaacagctgacatcaactaaaagcttcattaaatactttgaaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagctagcgcagcagcgacaaacggaacaatgatgcagtatttcgagtggtatgtacctaacgacggccagcaatggaacagactgagaacagatgccccttacttgtcatctgttggtattaacgcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaatcagcggcgaatataatattcaggcatggacaggcttcaactttccgggcagaggaacaacgtattctaactggaaatggcagtggttccattttgatggaacggattgggaccagagcagaagcctctctagaatcttcaaattcgatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggatattacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatccttctgtattttatggagatatgtacggtacaaaaggaacgacaacaagagagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaataaggaatcagtttcagtatgggtgcagcaatgaaagcttctcgaggttaacagaggacggatttcctgaaggaaatccgtttttttatttt

SEQ ID NO: 141 - DNA, 1759 bp, synthetic construct
  misc_feature 1..1759: note = amylase expression cassette
  source 1..1759: mol_type = other DNA
gtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagctagcgcagccgcaccgtttaacggtaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatatggaacaaaagctcaatatcttcaagccattcaagccgcccacgccgctggaatgcaagtgtacgccgatgtcgtgttcgaccataaaggcggcgctgacggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgattgggacgaaagccgaaaattaagccgcatttacaaattcaggggcatcggcaaagcgtgggattggccggtagacacagaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacaaacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgcttcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaaggggtcactgaaaaaccaggatccgggctggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgacctaaaagcttctcgaggttaacagaggacggatttcctgaaggaaatccgtttttttatttt

SEQ ID NO: 142 - DNA, 1581 bp, synthetic construct
  misc_feature 1..1581: note = expression cassette, synthetic sequence
  source 1..1581: mol_type = other DNA
gtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagcttcagcagctgatcaagcaggaaaaagcccggcaggcgtcagatatcatggcggtgatgaaatcatccttcagggctttcattggaacgtcgtcagagaagcgccgtataactggtataacatcctgagacaacaagcgagcacaattgccgctgatggcttttccgcaatctggatgccggttccgtggagagattttagcagctggacggatggagataaaagcggaggcggcgaaggatatttttggcatgactttaacaaaaacggccgctatggaagcgatgctcaactgagacaagcagcaggagcacttggaggagcaggagtcaaagtcctgtacgatgtcgtcccgaaccatatgaaccgcttttatccggacaaagaaatcaatctgccggcaggccaaagattttggagaaacgattgcccggacccgggaaatggaccgaatgattgcgatgatggcgatagatttctgggcggcgaagcggatctgaatacaggccatccgcaaatctatggcatgtttcgggacgaatttacgaatctgagaagcggatatggagcgggcggatttcgctttgattttgtcagaggctatgccccggaaagagttgatagctggatgagcgattcagcggatagcagcttttgcgtcggcgaactttggaaagaaccgagcgaatatccgccgtgggattggagaaatacagcgagctggcagcagatcatcaaagattggagcgatagagcaaaatgcccggtctttgactttgccctgaaagaacgcatgcaaaatggaagcgtcgccgattggaaacatggcctgaacggaaatccggacccgagatggagagaagtcgccgtcacgtttgtcgataaccatgacacaggatatagcccgggacaaaatggaggacaacataaatggccgcttcaagatggccttatcagacaggcgtatgcctatatccttacatcaccgggaacaccggttgtttattggccgcatatgtatgattggggctatggcgatttcatccgccaactgatccaggttagaagaacagcaggagtcagagcggatagcgccattagctttcatagcggctatagcggacttgtcgctacagttagcggcagccaacaaacactggtcgtcgccctgaatagcgatctggcaaatccgggacaagttgctagcggcagctttagcgaagcagtcaatgccagcaatggccaagtcagagtctggagaagcggaagcggagatggaggaggaaatgacggcggctaaaagagcagagaggacggatttcctgaaggaaatccgtttttttatttt

SEQ ID NO: 143 - DNA, 1452 bp, synthetic construct
  misc_feature 1..1452: note = Synthetic Cytophaga sp. variant #2 amylase expression cassette
  source 1..1452: mol_type = other DNA
gcagcgacaaacggaacaatgatgcagtatttcgagtggtatgtacctaacgacggccagcaatggaacagactgagaacagatgccccttacttgtcatctgttggtattacagcagtatggacaccgccggcttataagggcacgtctcaagcagatgtggggtacggcccgtacgatctgtatgatttaggcgagtttaatcaaaaaggtacagtcagaacgaagtatggcacaaaaggagaacttaaatctgctgtcaacacgctgcattcaaatggaatccaagtgtatggtgatgtcgtgatgaatcataaagcaggtgctgattatacagaaaacgtaacggcggtggaggtgaatccgtctaatagatatcaggaaacgagcggcgaatataatattcaggcatggacaggcttcaactttccgggcagaggaacaacgtattctaactggaaatggcagtggttccattttgatggaacggattgggaccagagcagaagcctctctagaatcttcaaattccatggaaaggcgtgggactggccggtttcttcagaaaacggaaattatgactatctgatgtacgcggactatgattatgaccatccggatgtcgtgaatgaaatgaaaaagtggggcgtctggtatgccaacgaagttgggttagatggatacagacttgacgcggtcaaacatattaaatttagctttctcaaagactgggtggataacgcaagagcagcgacgggaaaagaaatgtttacggttggcgaatattggcaaaatgatttaggggccctgaataactacctggcaaaggtaaattacaaccaatctctttttgatgcgccgttgcattacaacttttacgctgcctcaacagggggtggagcgtacgatatgagaaatattcttaataacacgttagtcgcaagcaatccgacaaaggctgttacgttagttgagaatcatgacacacagcctggacaatcactggaatcaacagtccaaccgtggtttaaaccgttagcctacgcgtttattctcacgagaagcggaggctatcctgcggtattttatggagatatgtacggtacaaaaggaacgacaacatatgagatccctgctcttaaatctaaaatcgaacctttgcttaaggctagaaaagactatgcttatggaacacagagagactatattgataacccggatgtcattggctggacgagagaaggggactcaacgaaagccaagagcggtctggccacagtgattacagatgggccgggcggttcaaaaagaatgtatgttggcacgagcaatgcgggtgaaatctggtatgatttgacagggaatagaacagataaaatcacgattggaagcgatggctatgcaacatttcctgtcaatgggggctcagtttcagtatgggtgcagcaatga

SEQ ID NO: 144 - DNA, 846 bp, synthetic construct
  misc_feature 1..846: note = synthetic allele glcT1 sequence
  source 1..846: mol_type = other DNA
gtgaacagatcctttaccgttgaaaaggtactcaacaacaacgttttaatcgctctccatgatgattacagagaagttgtcttgattggcaaaggaatcggttttggtaaaaagcgcggagatcttatcgaacatgagaactacgaaaaaatgtttatcctggaaaatgataaggaacaatcgcagtataagaagctcttcacttatgtcgatgaaaaaatggttgatatcgccaatgatgtcatctaccatatcgcgcaaaaaatcggccagccgctgaacgaacacattcatgtcgccctgacggaccatatcgcatttgcagttaagcgtctagaaaagggatttgatatgaaaaatccgtttttgcttgagacggaatcgctttatccgaaggaatacgaagtcgccaaggaagccgtcgatatgattaatgaaaaatccgacattcagctgcctgaaggtgaaatcgggttcatcgcgcttcatatccacagtgcgatgacaaaccgcccgctttctgaagtcaatcagcattcacaactgatctccaggcttgtccaggtcatcgaagattcattccagatgcaagtcaacagggaaagcgtgaactatttgcggctgatcaggcacttgcgctttacgattgacaggataaaacgggacgagccgattcaggaaccggaaaaattaatgttgttgttgaaaacggaatatccgctgtgttacaatactgcttggaagatgatcaagatcttgcagcaagcgctcaagaaaccggttcatgaggcagaagccgtttatttgacattgcatttgtaccgtttgactaataaaatttcataa